(19) United States
(12) Patent Application Publication
(10) Pub. No.: US 2015/0359585 A1
(43) Pub. Date: Dec. 17, 2015

(54) APPARATUS, SYSTEMS, AND METHODS FOR DISSECTION AND MODIFICATION OF TISSUES

(71) Applicant: Paul J. Weber, Queenstown (NZ)

(72) Inventor: Paul J. Weber, Queenstown (NZ)

(73) Assignee: TDM SurgiTech, Inc., Palm Harbor, FL (US)

(21) Appl. No.: 14/301,609

(22) Filed: Jun. 11, 2014

(51) Int. Cl. A61B 18/14

(52) U.S. Cl. CPC A61B 18/14; A61B 2018/00589

(57) ABSTRACT

Apparatus for tissue separation and/or modification. In some embodiments, the apparatus comprises a tip comprising a plurality of protrusions and at least one lysing element positioned between at least two adjacent protrusions. The apparatus may further comprise a spot coagulator comprising a spot coagulator tip for delivering energy for coagulating a blood vessel during a surgical procedure. The spot coagulator tip may be movable with respect to the plurality of protrusions to allow a surgeon to selectively deliver coagulating energy as desired.

### RELATED APPLICATIONS

This application claims the benefit under 35 U.S.C. §119(e) of U.S. Provisional Patent Application No. 61/840,406 filed Jun. 27, 2013 and titled “APPARATUS, SYSTEMS, AND METHODS FOR DISSECTION AND MODIFICATION OF TISSUES” which application is incorporated herein by reference in its entirety.

This application is also a continuation in part of U.S. patent application Ser. No. 13/659,873 filed Oct. 24, 2012, titled “MINIMALLY INVASIVE ECCRINE GLAND INCAPACITATION APPARATUS AND METHODS”, which claims the benefit of U.S. Provisional Application No. 61/659,295 filed Jun. 13, 2012.

This application is also a continuation in part of U.S. patent application Ser. No. 13/661,335 filed Oct. 26, 2012, titled “MINIMALLY INVASIVE APOCRINE GLAND INCAPACITATION APPARATUS AND METHODS”, which claims the benefit of U.S. Provisional Application No. 61/659,300 filed Jun. 13, 2012.

This application is also a continuation in part of U.S. patent application Ser. No. 13/759,969 filed Feb. 5, 2013, titled “SYSTEMS, APPARATUS AND METHODS FOR TISSUE DISSECTION AND MODIFICATION”, which claims the benefit of U.S. Provisional Application No. 61/748,037 filed Dec. 31, 2012 and which claims the benefit of U.S. Provisional Application No. 61/751,239 filed Jan. 10, 2013.

This application is also a continuation in part of U.S. patent application Ser. No. 13/759,997 filed Feb. 5, 2013, titled SYSTEMS, APPARATUS AND METHODS FOR TISSUE DISSECTION AND MODIFICATION, which claims the benefit of U.S. Provisional Application No. 61/748,037 filed Dec. 31, 2012 and which claims the benefit of U.S. Provisional Application No. 61/751,239 filed Jan. 10, 2013.

This application is also a continuation in part of U.S. patent application Ser. No. 13/760,042 filed Feb. 5, 2013, titled METHODS, APPARATUS, AND SYSTEMS FOR TISSUE DISSECTION AND MODIFICATION, which claims the benefit of US Provisional Applications Nos. 61/748,037 filed Dec. 31, 2012 and 61/751,239 filed Jan. 10, 2013.

This application is also a continuation in part of U.S. patent application Ser. No. 13/760,028 filed Feb. 5, 2013, titled “METHODS AND SYSTEMS FOR TISSUE DISSECTION AND MODIFICATION”, which claims the benefit of US Provisional Applications Nos. 61/748,037 filed Dec. 31, 2012 and 61/751,239 filed Jan. 10, 2013.

This application is also a continuation in part of U.S. patent application Ser. No. 13/802,731 filed Mar. 14, 2013, titled “APPARATUS AND SYSTEMS FOR TISSUE DISSECTOIN AND MODIFICATION, which claims the benefit of US Provisional Applications Nos. 61/748,037 filed Dec. 31, 2013, 61/751,239 filed Jan. 10, 2013, 61/760,628 filed Feb. 4, 2013, and further is a continuation in part of Ser. No. 13/759,969 filed on Feb. 5, 2013.

Each of the foregoing patents is hereby incorporated by reference in its entirety.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1a is a perspective view of an embodiment of a modular tissue dissector illustrating the protrusions and lysing elements, wherein some of the protrusions and lysing elements are oriented in a non-axial direction.

FIG. 1b is a side view of the embodiment previously depicted in FIG. 1a.

FIG. 1c is an upper plan view of the embodiment previously depicted in FIG. 1a also depicting a spot coagulating lysing element.

FIG. 1d is an upper plan view of the embodiment previously depicted in FIG. 1a reversibly attached to a rigid shaft of an electrosurgical ‘pencil’.

FIG. 1e is a cross sectional view of the rigid shaft portion of the embodiment depicted in FIG. 1d.

FIG. 2a is an upper plan view of an embodiment of a modular tissue dissector and modifier with an electrosurgical energy window on the upper side of the device.

FIG. 2b is a perspective view of the embodiment previously depicted in FIG. 2a reversibly attached to a rigid electrosurgical ‘pencil’ shaft.

FIG. 3 is an upper plan view of an embodiment of a modular tissue dissector illustrating the protrusions and lysing elements, wherein the protrusions and lysing elements are oriented only in non-axial directions.

FIG. 4 is an upper plan view of an embodiment of a modular tissue dissector illustrating the protrusions and lysing elements, wherein the protrusions and lysing elements are oriented in axial and non-axial directions.

FIG. 5a is an upper plan view illustrating the protrusions and lysing elements of an embodiment of a modular tissue dissector lacking a spot coagulating lysing element, wherein the protrusions and lysing elements are oriented in axial and non-axial directions.

FIG. 5b is an upper plan view of the embodiment previously depicted in FIG. 5a reversibly attached to a rigid electrosurgical shaft.

FIG. 6a is an upper plan view illustrating an embodiment of a modular tissue dissector, wherein some of the protrusions and lysing elements are oriented in a non-axial direction with an attached axially-slidable spot coagulator.

FIG. 6b is a side view of the embodiment previously depicted in FIG. 6a.

FIG. 7a is an upper plan view illustrating an embodiment of a tissue dissector, wherein some of the protrusions and lysing elements are oriented in a non-axial direction with a rotationally deployable spot coagulation boom.

FIG. 7b is a side view of the embodiment previously depicted in FIG. 7a.

FIG. 8a is an upper plan view illustrating an embodiment of a tissue dissector wherein the protrusions and lysing elements are oriented in an axial direction with an attached axially-slidable spot coagulator.

FIG. 8b is a side view of the embodiment previously depicted in FIG. 8a.

FIG. 9a is an upper plan view illustrating an embodiment of a tissue dissector, wherein the protrusions and lysing elements are oriented in an axial direction with an attached rotationally deployable spot coagulation boom.

FIG. 9b is a side view of the embodiment previously depicted in FIG. 9a.

FIG. 10a is an upper plan view illustrating an embodiment of a tissue dissector wherein some of the protrusions and lysing elements are oriented in a non-axial direction with an attached axially-slidable spot coagulator, spot coagulator sheath, and spot coagulator connecting conductor cable.

FIG. 10b is a side view of the embodiment previously depicted in FIG. 10a

FIG. 11a is an upper plan view illustrating an embodiment of a tissue dissector wherein the protrusions and lysing elements are oriented in an axial direction with an attached axially-slidable spot coagulator, spot coagulator shield, and spot coagulator connecting conductor cable.

FIG. 11b is a side view of the embodiment previously depicted in FIG. 11a

FIG. 12a is an upper plan view illustrating an embodiment of a tissue dissector wherein some of the protrusions and lysing elements are oriented in a non-axial direction with an attached axially-slidable spot coagulator and spot coagulator toggle.

FIG. 12b is a side view of the embodiment previously depicted in FIG. 12a

FIG. 13a is an upper plan view illustrating an embodiment of a tissue dissector wherein the protrusions and lysing elements are oriented in an axial direction with an attached axially-slidable spot coagulator and spot coagulator toggle.

FIG. 13b is a side view of the embodiment previously depicted in FIG. 13a

FIG. 14a is a perspective view of an embodiment of a tissue dissector and modifier with an energy window on the upper side of the device and a handle.

FIG. 14b is a side elevation view of the embodiment previously depicted in FIG. 14a.

FIG. 15a is a wiring diagram of one embodiment of the TDM employing two switches.

FIG. 15b is a wiring diagram of another embodiment of the TDM employing one switch.

FIG. 15c is a wiring diagram of another embodiment of the TDM employing one switch.

FIG. 15d is a wiring diagram of a circuit in another embodiment of the TDM designed to modulate the energy delivered.

FIG. 16 is a wiring diagram of a bipolar embodiment of the TDM

FIG. 17a is a side view of a robotic surgery system comprising a TD.

FIG. 17b depicts an alternative robotic arm that may be used with the system of FIG. 17a.

FIG. 18 is a flow chart illustrating one implementation of a method of an energy emission—sensor feedback loop.

FIG. 19 is a flow chart illustrating one implementation of a method for accessing an organ using the TDM.

FIG. 20 depicts an embodiment comprising a shaft having a flexible segment.

FIG. 21 is a flow chart illustrating one implementation of a method for separating and/or modifying tissue using a TD.

FIG. 22a is an upper plan view illustrating an embodiment of a bipolar tissue dissector wherein the protrusions and lysing elements are oriented an axial direction and return electrode(s) comprise lysing elements.

FIG. 22b is an upper plan view that depicts an embodiment of wiring to the lysing elements of the embodiment previously depicted in FIG. 23a.

FIG. 22c is a side view of a bipolar circuit activating foot-pedal bypass comprising a sticker.

FIG. 22d is a side view of the embodiment previously depicted in FIG. 23a with the bipolar circuit activating foot-pedal bypass sticker applied.

FIG. 23 is a perspective view of a bipolar circuit activating foot-pedal bypass comprising an adhesive backed pad.

FIG. 24a is a side view of a bipolar circuit activating foot-pedal bypass comprising a sheath.

FIG. 24b is a side view of a bipolar tissue dissector ensheathed in bipolar circuit activating foot-pedal bypass comprising a sheath.

FIG. 25a is an upper plan view illustrating an embodiment of a modular common intermediate configuration tissue dissector wherein the protrusions and lysing elements are oriented an axial direction and return electrode is not located on a lysing segment.

FIG. 25b is a side view of the embodiment previously depicted in FIG. 25a.

FIG. 26a is an upper plan view illustrating an embodiment of a tissue dissector wherein some of the protrusions and lysing elements are oriented in a non-axial direction with an attached axially-slidable spot coagulator, spot coagulator toggle and spot coagulator moving means which is rigidly affixed to an electrosurgical ‘pencil’ shaft.

FIG. 26b is a side view of the embodiment previously depicted in FIG. 26a.

FIG. 27 is an upper plan view illustrating an embodiment of a tissue dissector wherein some of the protrusions and lysing elements are oriented in a non-axial direction with an attached axially-slidable spot coagulator, spot coagulator toggle and spot coagulator moving means which is rigidly affixed to a modular tissue dissector shaft.

FIG. 28a is an upper plan view illustrating an embodiment of a tissue dissector tip wherein the protrusions and lysing elements are oriented in a axial direction with an attached slidable spot coagulator passing through a TD tip passageway.

FIG. 28b is a side view of the embodiment previously depicted in FIG. 28a.

FIG. 28c is an upper plan view illustrating an embodiment of a tissue dissector wherein some of the protrusions are oriented in an axial direction with a TD tip passageway (containing an axially-slidable spot coagulator in a storage position within the TD tip), spot coagulator toggle and spot coagulator moving means which is rigidly affixed to an electrosurgical ‘pencil’ shaft.

FIG. 28d is a side view of the embodiment previously depicted in FIG. 28d with the spot coagulator deployed in the operational position.

FIG. 28e is an upper plan view illustrating an embodiment of a spot coagulator shaft & SC tip wherein the SC tip may resemble a lysing element.

FIG. 29 is a flow chart of an implementation of a method for making an electrosurgical incision using the lysing elements of a tissue dissector.

FIG. 30a is a perspective view of another embodiment of a modular TDM comprising a spot coagulator coupled with a shaft of an electrosurgical instrument.

FIG. 30b is a perspective view of a conductive shaft of the modular TDM of FIG. 30a.

FIG. 30c is a close-up perspective view of the tip of the modular TDM of FIG. 30a.

### DETAILED DESCRIPTION

The term dissection may indicate the separation of tissues or of one tissue plane from another (ref: Free Online Medical Dictionary). Some also consider dissection to comprise separation of a single tissue into portions. Much of the bodies of animals and humans are formed from embryonic fusion planes. Many of the organs of the human body are categorized from the embryonic fusion planes from whence they came. The interfaces between organs may often be referred to as ‘tissue planes.’ Such planes may be considered substantially planar depending upon the size of a comparative planar living or inanimate object (such as a surgical instrument). The TD and/or TDM may perform the functions of sharp dissection, blunt dissection and electrosurgical cutting and/or coagulation simultaneously without a surgeon having to switch instruments. Sharp dissection has been referred to by some as separation of tissues by means of the sharp edge of a knife or scalpel or with the inner sharp edge of scissors. Blunt dissection has been defined by Webster as surgical separation of tissue layers by means of an instrument without a cutting edge or by the fingers.

The term ‘minimally invasive surgery’ has been used to describe a procedure (surgical or otherwise) that is less invasive than open surgery used for the same purpose. Some minimally invasive procedures typically involve use of laparoscopic devices and remote-control manipulation of instruments with indirect observation of the surgical field through an endoscope or similar device, and are carried out through the skin or through a body cavity or anatomical opening. This may result in shorter hospital stays, or allow outpatient treatment (reference: Wikipedia).

The term ‘open surgery’ is used to indicate cutting skin and tissues to ‘open the body’ so that the surgeon has direct access to the structures or organs involved. Often an incision is made of a size that permits the surgeon's hands entry into a patient's body. The structures and tissues involved can be seen and touched, and they are directly exposed to the air of the operating room.

The term Tissue Dissector (TD) is intended to encompass any of the devices for dissecting tissue disclosed herein including but not limited to Tissue Dissecting and Modifying Wands (TDM) comprising lysing elements, tissue dissecting and modifying wands lacking lysing elements, and tissue dissecting wands either comprising or lacking energy windows. In some embodiments, the lysing elements may comprise lysing segments.

The term ‘modifying’ in this context may refer to or may encompass application of energy to living tissue using one or more lysing elements as discussed herein. The term ‘modifying’ in this context may also refer to application of energy to tissue by way of an energy window as also described herein. Such methods may be performed using a Tissue Dissecting and Modifying Wand (“TDM”). Examples of various embodiments of such wands may be found in U.S. Pat. No. 6,203,540 titled “Ultrasound and Laser Face-Lift and Bulbous Lysing Device,” U.S. Pat. No. 6,391,023 titled “Thermal Radiation Facelift Device,” U.S. Pat. No. 6,432,101 titled “Surgical Device for Performing Face-Lifting Using Electromagnetic Radiation,” U.S. Pat. No. 6,440,121 titled “Surgical Device For Performing Face-Lifting Surgery Using Radiofrequency Energy,” U.S. Pat. No. 6,974,450 titled “Face-Lifting Device,” and U.S. Pat. No. 7,494,488 titled “Facial Tissue Strengthening and Tightening Device and Methods.” The “Detailed Description of the Invention” section of each of these patents is hereby incorporated herein by specific reference. With respect to U.S. Pat. No. 6,203,540 titled “Ultrasound and Laser Face-Lift and Bulbous Lysing Device,” the section titled “Description of the Preferred Embodiments” is hereby incorporated herein by specific reference.

Some tissues and/or organs and/or tumors treated with the TDM may be of varying sensitivity to electrosurgical and/or other forms of energy. Modulation and feedback may be helpful for such tissues. Controlling the amount of energy delivered by a moving device within the body that is continuously delivering energy and/or delivering energy in a pulsed format may rely upon methods to locate the device in real time, and/or the change in position of the device relative to a tissue or portion of the tissue over time. Real time calculations may be made by hardware and/or software to record and/or control energy delivery. For example, an antenna system in relation to Tissue Dissecting Wands and/or Tissue Dissection and Modifying Wands has been previously described by Weber in U.S. patent application Ser. No. 13/802,731 titled: Apparatus and Systems for Tissue Dissection and Modification & U.S. patent application Ser. No. 13/767,876 titled: System, Apparatus and Methods for Tissue Dissection which are hereby incorporated by reference in their entirety. Although a common laser operated computer mouse may be used to track distances moved over time on surface skin with limited degree of accuracy, such a system may be unsuitable to track distanced moved when placed on a primarily fatty undersurface of the skin (i.e. applied from within the body). This may be related to the inability of such a device to detect a speckle pattern in tissue that may share similarities to butter such as reflectance and homogeneity. Herein to be discussed will be solutions to ‘tracking’ movement against internal organs and/or tissues and/or the undersurface of the body skin.

The TD and/or TDM may deposit energy in tissues via for example energized lysing elements, one or more energy windows or other such components on the TD or TDM and thus heat tissue to a measurable level. The speed of passage of the TD and/or TDM may influence the amount of energy deposited by said lysing elements, energy window or other such energy delivering element. Thus, methods of reasonable accuracy to determine the speed of motion of the area(s) emitting the energy may with other determined parameters (such as power output per unit area) permit real time or delayed calculation of the power delivered per unit area for a given exposure time. For real time calculations, a feedback loop may be created to control energy emissions (amount, frequency, intensity, etc).

The TDM may be “energized” by various forms of energy in its top side energy window, as described in greater detail below. Such energy absorptions may result in the formation of heat which may, in turn, denature tumor and/or other tissue cells themselves, and/or their surrounding environment in order to achieve a desired effect of a surgical method or procedure.

In some embodiments, energy may be delivered from one or more energy windows so as to heat tissue to a given temperature range. Various methods may therefore be implemented in which the amount of energy and/or the delivery time may be adjusted so as to heat the tissue to within a desired temperature range. Temperature sensors may therefore be incorporated on or near the energy windows to allow a surgeon to heat the tissue to a desired temperature or within a desired temperature range. In some embodiments, the sensor may be configured to provide an average temperature over a particular period of time and or over a particular range of distances within the tissue. Systems consistent with the disclosure provided herein may be configured to prevent or to shut down or otherwise limit energy transfer if a particular tissue temperature were beyond a threshold or alternatively if an average temperature threshold is reached.

Temperature sensors that may be useful in connection with embodiments disclosed herein include, but are not limited to, resistance temperature sensors, such as carbon resistors, film thermometers, wire-wound thermometers, or coil elements. Some embodiments may comprise thermocouples, pyrometers, or non-contact temperature sensors, such as total radiation or photoelectric sensors. In some embodiments, one or more temperature sensors may be coupled with a processor and/or a monitor to allow a surgeon to better visualize or otherwise control the delivery of energy to selected areas of target tissue. For example, some embodiments may be configured such that a surgeon can visualize the temperature of tissue positioned adjacent to one or more locations along the TD and/or TDM to ensure that such temperatures are within a desired temperature range. Some embodiments may alternatively, or additionally, be configured such that one or more temperature sensors are coupled with a processor in a feedback loop such that energy delivery may be automatically adjusted by the system in response to temperature data. For example, when temperatures exceed a particular threshold, such as somewhere between about 65° C. and about 90° C., the system may be configured to shut down or otherwise limit further energy delivery. In some such embodiments, the threshold may be between about 68° C. and about 75° C. Because the TDM is capable of delivering energy in a fractionated pattern, temperature thresholds and or ranges may vary depending upon the size of the spot being measured and/or the proximity and/or recency (time) of the spot being measured to the treatment area. Thus, some embodiments and implementations may comprise a threshold lesser or greater than those referenced above.

Some embodiments may comprise a feedback means, such as a visual, audible, or tactile feedback means, to provide information to a user to avoid excess energy delivery to tissues. In some embodiments, the feedback means may be configured to notify the surgeon when the temperature has reached a particular threshold. In some embodiments, the feedback means may be configured to notify the surgeon when the TD and/or TDM have been positioned in a particular location within the target region for a particular time period. Examples of visual feedback means include LED lights, LASERS, visual light source, display screen, etc. Examples of audible feedback means include speakers, alarms, audible vibration, etc. Examples of tactile feedback means include vibration, minimal electrical shock, heat, etc. The feedback means may be configured with multiple thresholds with different feedback at each threshold. For example, at a first threshold, the TD and/or TDM may be configured to deliver a first noise and at a second threshold the TD and/or TDM may be configured to deliver a second noise. The second noise may be louder than the first noise to indicate a greater urgency for changing the energy delivery and/or moving the TD and/or TDM from its current location within a patient's body. In some embodiments, an antenna(s) may be present on the shaft or tip of the TD and/or TDM. In some embodiments, a camera or fiberoptic may gather optical data to allow the surgeon knowledge of the placement of the TD and/or TDM. During some open surgeries, surgeons using the TD and/or TDM may lyse a vessel that exceeds the hemostatic capabilities of a given TD and/or TDM. Even though an isolated electrode from the ‘upper’ plane energy window may be inverted to coagulate said vessel, it may be more advantageous to have a spot coagulator extend from an embodiment of the TD and/or TDM at such a distance and/or location that allows complete viewing and/or contact of the bleeding area with a portion of the spot coagulator (for example the distal end point of a tip of the coagulator). Such a probe may be deployable and obtain electrical energy off of a conductive element located between the lysing elements of the tip and the plug.

Further details regarding various embodiments will now be provided with reference to the drawings.

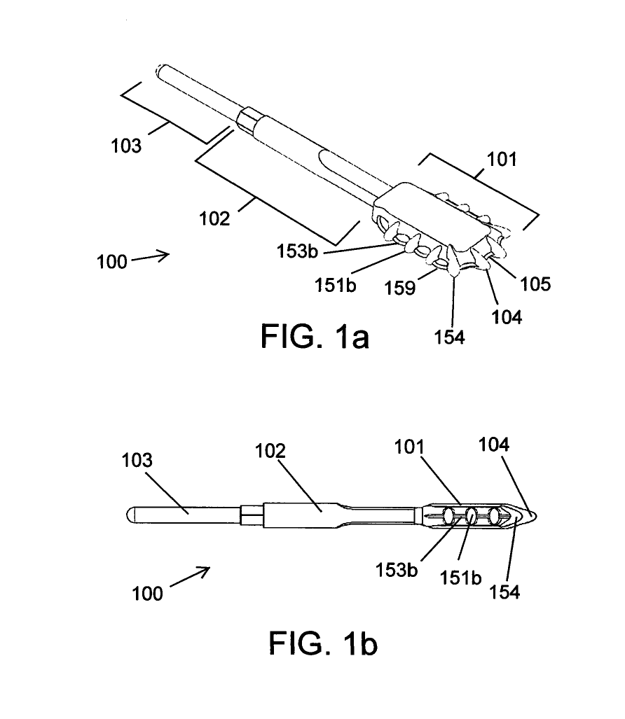

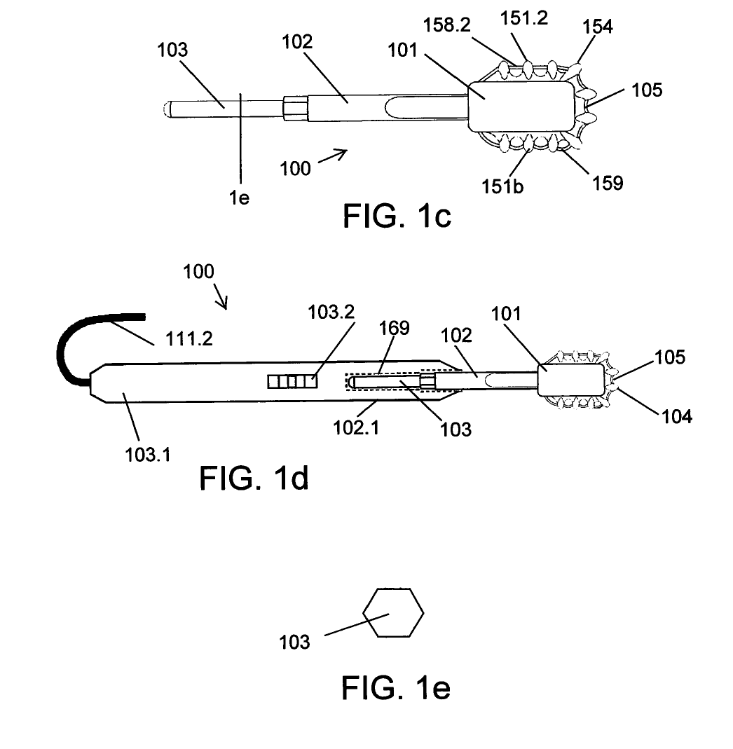

FIGS. 1a,b depict an embodiment of a modular Tissue Dissector (modular TD) 100 comprising a modular TD tip 101, a modular TD shaft 102 and a modular TD plug 103, wherein some of the protrusions and lysing elements are oriented in non-axial directions. In other words, protrusions 151b are not oriented in a direction of the axis of modular TD shaft 102. In the depicted embodiment, modular TD shaft 102 may comprise an insulating and/or supportive coating and/or cover overlying a conductive material and/or metal axial core. The embodiment depicted in FIGS. 1a&b also comprises transitional protrusions 154. Transitional protrusions 154 extend from opposing corners (apices) of modular TD tip 101 and extend in directions at least approximately at midpoint between an axial direction and a direction perpendicular to the axial direction as indicated by protrusion 151b. Transitional protrusions may be useful in preventing the device from becoming entangled in dense tissue. This embodiment also comprises a corner (or transitional) protrusion 154 that extends at a transitional angle relative to axial protrusions 104 and non-axial side protrusion 151b. In some embodiments, one or more transitional angles may be acute. In some embodiments, one or more transitional angles may be obtuse.

In this embodiment, non-axial protrusion 151b extends in a direction that is at least substantially perpendicular to the direction in which axial protrusions 104 extend. More particularly, there are two sets of non-axial protrusions (one set is depicted on the right side and one set is depicted on the left side of the embodiment of FIG. 1a). Both sets of non-axial protrusions extend in directions that are at least substantially perpendicular to the direction in which axial protrusions 104 extend (namely, along a longitudinal axis of the TDM shaft). In addition, it can be seen in FIG. 1a that the two sets of non-axial protrusions extend in directions that are at least substantially opposite from one another.

In some embodiments, axial protrusions 104 may extend at least substantially along a longitudinal axis of the shaft, as described above. In some embodiments non-axial protrusion such as 151b may extend at an angle of between zero degrees and 30 degrees of a normal to the direction in which the axial protrusions 104 extend. It is contemplated that it may be desirable for some implementations and embodiments to provide non-axial tips extending in a direction or directions falling within this range in order to, for example, allow a surgeon to effectively perform both a to and fro, and a side-to-side (“windshield wiper”) motion using the TDM. As described above transitional protrusions may be useful to enable a surgeon to avoid entangling the dissector in tissue during one or both such motions.

In some embodiments, the modular TD tip can be a separate piece that is secured to the modular TD shaft by a variety of methods such as a snap mechanism, mating grooves, plastic sonic welding, etc. Alternatively, in some other embodiments, the modular TD tip can be integral or a continuation of a modular TD shaft made of similar metal or materials. In some embodiments, the modular TD tip may also be constructed of materials that are both electrically non-conductive and of low thermal conductivity; such materials might comprise, for example, porcelain, ceramics, glass-ceramics, plastics, varieties of polytetrafluoroethylene, carbon, graphite, and/or graphite-fiberglass composites, etc. In some embodiments, the modular TD tip may be constructed of a support matrix of an insulating material (e.g., ceramic or glass material such as alumina, zirconia). In some embodiments, the modular TD tip may comprise cermets.

In other contemplated embodiments, the modular TD tip may comprise polyether etherketone &/or polytetrafluoroethylene with hollow glass microspheres (3M, St. Paul, Minn.). External power control bundles as previously described in other embodiments may connect to electrically conductive elements to bring RF electrosurgical energy from an electrosurgical generator via, for example a handle and/or electrosurgical ‘pencil,’ down to the modular TD plug 103 (which is electrically connected) to modular TD shaft 102 to electrically conductive lysing elements, such as lysing elements 153b, mounted in the recessions in between the protrusions, such as protrusions 151b. In some embodiments, the protrusions may comprise bulbous protrusions. The tip shown in this embodiment has two relative protrusions 104 and one relative recession 105 pointing along the main axis of the TDM and provides for a monopolar tip conductive element; the tip shown also has eight protrusions pointing in non-axial directions as well as relative recessions pointing in non-axial directions. In other embodiments the modular TD tip may have one or more non-axial protrusions and one or more non-axial relative recessions. In some embodiments the tip may have between 3 and 100 non-axial protrusions and relative recessions. It should be understood that the number of protrusions need not match the number of lysing elements or recessions. In the depicted embodiment, the modular TD tip 101 may alternatively be made partially or completely of concentrically laminated or annealed-in wafer layers of materials that may include plastics, silicon, glass, glass/ceramics, cermets or ceramics. Lysing elements may also be made partially or completely of a cermet material. Alternatively, in a further embodiment the tip may be constructed of insulation covered metals or electroconductive materials. The lysing elements may be located at the termini of conductive elements. In some embodiments, lysing elements may be continuous and continue to extend all the way around a tip. In some embodiments lysing elements may be discrete and do not extend all the way around a tip. For example, in some embodiments each of the lysing elements may comprise a separate piece of material. In other embodiments, each of the lysing elements may comprise a portion of a single plate, wire or other piece of material.

In the depicted embodiment, modular TD tip 101 which terminates in protrusions, such as 104 and 151b, may comprise materials that are both electrically non-conductive and of low thermal conductivity such as porcelain, epoxies, ceramics, glass-ceramics, plastics, or varieties of polytetrafluoroethylene. Alternatively, the tip may be made from metals or electroconductive materials that are completely or partially insulated. In some embodiments, the electrically conductive tissue lysing elements(s), such as 153b, may have any geometric shape including a thin cylindrical wire, and may be positioned within the relative recessions of the tip. The electrically conductive lysing elements can be in the shape of a plate or plane or wire and made of any metal or alloy that does not melt under operating conditions or give off toxic residua. The electrically conductive lysing elements may comprise steel, nickel, alloys, palladium, gold, tungsten, silver, copper, and platinum.

In the depicted embodiment, modular TD tip 101, which terminates in protrusions such as 154, may have one or more convex lysing elements (some embodiments of which may be of a greater size than any or most other contained (contained between 2 protrusions) lysing elements) such as spot coagulation lysing elements 159. In some embodiments, the electrically conductive tissue lysing elements (s) 159 may have any geometric shape including a thin cylindrical wire, and may be positioned within any of the relative recessions of the tip. Again, during some open surgeries, surgeons using the TD and/or TDM may lyse a vessel that exceeds the hemostatic capabilities of a given TD and/or TDM. Even though an isolated electrode from the ‘upper’ plane energy window may be inverted to coagulate said vessel, it may be more advantageous to have spot coagulator coagulation capabilities within the same instrument. So as not to have the surgeon set down the TD or TDM, to pick up other instrumentation from the surgical tray to stem the bleeding blood vessel, it may be beneficial for the surgeon to have the option of ‘spot’ coagulation using the same dissecting TD or TDM. Thus, with a protruding lysing element, the surgeon may simply request operating personnel to increase the electrosurgical generator coagulation power (often in ‘coagulation’ current mode or ‘blend’ mode) while the surgeon angles the TD or TDM tip toward the bleeding vessel with the spot coagulation lysing element leading the way. The surgeon may then activate the electrosurgical generator while touching the prominent lysing element to the bleeding blood vessel. Carbonized debris remaining on the spot coagulation lysing element 159 may be removed using a nylon brush and/or surgical ‘scratch’ pad. Once the bleeding vessel has been stopped, the surgeon can request power to the electrosurgical generator be modified appropriately and may continue any remaining dissection with the TD or TDM. The spot coagulation lysing element may be 50% larger in surface area than similarly curved lysing elements and may vary from 20% larger to 70% larger.

In some embodiments, the shaft may be flat, rectangular or geometric in cross-section and/or substantially flattened. In some embodiments, smoothing of the edges of the shaft may reduce friction on the tissues surrounding the entrance wound. In some further embodiments, the shaft may be made of metal or plastic or other material with a completely occupied or hollow interior that can contain insulated wires, electrical conductors, fluid/gas pumping or suctioning conduits, fiber-optics, or insulation.

In some embodiments, shaft plastics, such as polytetrafluoroethylene, may act as insulation about wire or electrically conductive elements. In some embodiments, the shaft may alternatively be made partially or completely of concentrically laminated or annealed-in wafer layers of materials that may include plastics, silicon, glass, glass/ceramics, ceramics carbon, graphite, and/or graphite-fiberglass composites.

FIG. 1c is an upper plan view of the embodiment depicted in FIG. 1a of a modular TD 100 comprising a modular TD tip 101 with 3 different shaped lysing elements. Lysing element 105 is concave, lysing element 158.2 is straight as are the other lysing elements on the same side, lysing element 159 is convex, as are the other lysing elements on its same side. Non-axial protrusions 151b and 151.2 point in substantially opposite directions. In the depicted embodiment, the double lines on the lysing elements indicate a bevel that is sharpened. In other embodiments, the bevel is not required to be sharp. In other embodiments the lysing element may be a wire. In some embodiments, all the lysing elements are of the same shape. In some contemplated embodiments, one or more of the lysing elements may differ from the rest in shape and/or other characteristics. Lysing elements shapes may be selected depending upon the operating surgeon's concern of the for a variety of factors, including, but not limited to: level of cutting and/or aggressiveness of dissection and/or tissue type dissected and/or proximity to vital structures and/or intended speed of dissection and/or viability/condition of tissue being dissected (semi-dead burn tissue or living interface) and/or density and/or vascularity. For example, a convex lysing element may protrude beyond what a concave lysing element might protrude and may cut into or dissect tissue, such as fat, faster but may higher chance of cutting blood vessels (fortunately, nerves may be fewer in fat so the surgeon may be able to dissect faster). Another example may be that a straight blade may pass more readily though devitalized tissue, such as burn victim skin, as the tissue may become gelatinous and/or homogenous in consistency. Concave lysing elements may be beneficial for forward motion as the forces applied to bulbs on forward/axial tip motion may exceed those usually encountered on side/perpendicular-to-axial/′normal′ tip motion; thus, compaction of tissues at the relative recessions and/or lysing elements may beneficially exclude certain larger diameter blood vessels and/or nerves from being lysed (possibly depending upon the anatomic site and/or angle of attack).

FIG. 1d depicts an embodiment of a modular TD connected to an electrosurgical ‘pencil’ shaft 102.1, and an electrosurgical ‘pencil’ handle 103.1. Rocker switch 103.2 located on or about handle 103.1 and/or shaft 102.1 may control the electrosurgical energy/energies derived from an electrosurgical generator (not seen in this view) brought into the handle via conduit 111.2. The modular TD 100 is modular in that it is removable from shaft 102.1. More particularly, the modular TD comprises a means for removably coupling the modular TD with an electrosurgical pencil shaft 102.1 at modular TD plug 103. In the depicted embodiment, this coupling means comprises a modular TD plug 103. In some embodiments, modular TD plug 103 may be threaded to facilitate a secure coupling between modular TD 100 and shaft 102.1. However, in other embodiments, the coupling means may comprise a recess configured to receive a plug formed on the shaft. In still other embodiments, the coupling means may comprise a snap-fit coupling, a friction fit coupling, a bayonet clip, etc.

In the depicted embodiment, modular TD plug 103 is configured to be received within a corresponding recess 169 formed within shaft 102.1. In some embodiments, elements within modular TD plug 103 and/or recess 169 may be electrically connected with electrical elements within shaft 102.1 and/or handle 103.1 which are in turn connected with switch 103.2 and/or conduit 111.2. In some embodiments, modular TD plug 103 may be configured to electrically couple the modular TD with electrosurgical pencil shaft 102.1. In this manner, in embodiments comprising, for example, electricity from a power source may be transmitted through the coupling between modular TD plug 103 and recess 169 to allow for energizing the lysing elements.

In some embodiments, modular TD 100 may be disposable as well, such that a surgeon can place an appropriate modular TD on the shaft and remove and dispose of the modular TD after surgery. Alternatively or additionally, a plurality of different modular TDs may be provided, each of which may be disposable, or may be configured for sterilization and re-use, and an appropriate modular TD may be selected as needed for a particular surgery.

In the depicted embodiment, modular TD tip 101 comprises a plurality of protrusions 104, some of which are non-axial, and a plurality of recessions 105 positioned therebetween, as described above. In some embodiments a modular TD comprising a tip comprising only axial protrusions may be swapped for modular TD 100 as desired to suit a particular surgical procedure.

In an embodiment, the modular TD tip 101 may measure about 12 mm in width, about 15 mm in length and about 5 mm in maximal thickness; the modular TD shaft 102 may measure about 4 mm in diameter and/or about 20 mm in length; the modular TD plug 103 may measure about 2.5 mm in diameter and about 20 mm in length; the overall modular TD 100 may measure about 12 mm in width, 55 mm in length and about 5 mm in maximal thickness. Embodiments are contemplated wherein sizes of about one-fifth to about five times these dimensions may have possible uses. It is also contemplated, for example in some veterinary embodiments, tip sizes of about one-tenth to 20 times the aforementioned dimensions may also have possible uses. In some embodiments wherein electrical insulation and/or polymeric insulating coating is present on such parts, for example modular TD shaft 102, such insulation may measure about 0.5 mm in thickness; in some contemplated embodiments, the insulation thickness may range from 0.1 mm to 3 mm. In some embodiments, insulations may comprise materials that are both electrically non-conductive and of low thermal conductivity; such materials might comprise, for example, porcelain, ceramics, glass-ceramics, plastics, various halogenated carbon molecules, polytetrafluoroethylene, carbon, graphite, and graphite-fiberglass composites and the like.

In some embodiments, wherein the modular TD plug 103 may be manufactured with a circular cross section to plug into standard electrosurgical pencils with a corresponding circular cross section, it may be possible that torque forces resulting from moving the modular TD tip 101 through tissue may result in loosening of the fit and/or modular TD 100 rotations. In some embodiments, the TD plug 103 connection may be made tighter by things such as: screw threads and/or gnarling and/or cross-hatchings and/or a conductive textured surface and/or a jacket and/or a form-fit and/or snap-fit and/or an adhesive and/or a spear prong and/or macro or microscopic surface texture change and/or cross-sectional geometry and/or a ‘jacket’ and/or a hole (through and through, for a wire to be passed and spiral twisted to mimic a screw thread), etcetera in one or more portions of the receiving areas or the giving portions of the plug; such things may prevent rotation of the tip in/on certain devices. In contemplated embodiments, material may be welded onto one or more portions of the receiving areas and or giving portions of the plug to reduce and/or prevent rotation.

In some embodiments, loosening may be minimized by a brief polygonal cross section (sometimes comprised of a plastic and/or insulated surface zone) in the outer, more proximal surface layer of the shaft that fits that matches a manufactured corresponding receiving area in models of current ‘pencils’ (for example those made by ValleyLab).

FIG. 1e is a cross sectional view of line marking ‘le’ of the modular TD plug 103 found in FIG. 1c. As shown in FIG. 1e, in some embodiments, modular TD plug 103 may have a non-circular cross sectional shape, for example, in the embodiment shown in FIG. 1e comprises a hexagonal cross sectional shape however other polygonal cross sectional shapes comprising any number of flat or curved surfaces as desired. Some embodiments may also comprise a jacket configured to be received over a plug so as to, for example, transform a plug having a circular cross section into a plug having an alternative shape. Some embodiments comprising a hexagonal or other polygonal cross sectional shape may be useful for allowing a surgeon to orient the TD during a surgical procedure.

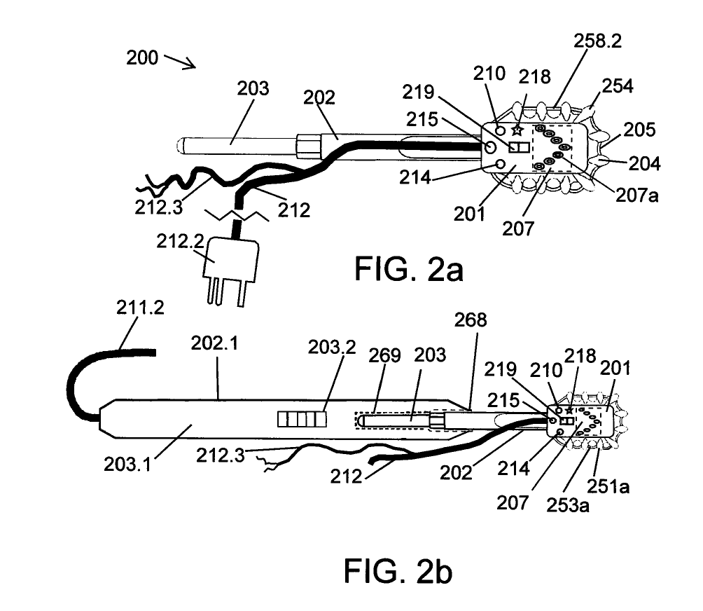

FIGS. 2a,b depict an embodiment of a modular Tissue Dissector & Modifier (modular TDM) 200 comprising a modular TDM tip 201, a modular TDM shaft 202 and a modular TDM plug 203, wherein some of the protrusions and lysing elements are oriented in non-axial directions. In other words protrusions, such as 251a, are not oriented in a direction of the axis of modular TDM shaft 202. In the depicted embodiment, modular TDM 200 comprises energy window 207 which may comprises electromagnetic energy emitting elements. In the depicted embodiment, elements 207a may be configured to emit radiofrequency/electrosurgical energy such as RF energy. Energy window 207 may be configured with a plurality of energy emitting elements 207a. In some such embodiments energy delivering elements 207a may be spaced apart so as to provide for areas of energy delivery and interspersed areas of tissue sparing. It should be noted that the term “energy window” is intended to encompass what is referred to as a planar-tissue-altering-window/zone in U.S. Pat. No. 7,494,488 and, as described herein, need not contain radiofrequency/electrosurgical energy emitting elements in all embodiments Additionally, the “energy window” may comprise a variety of other energy emitting devices, including but not limited to radiofrequency, microwave, light, intense pulsed light, LASER, thermal, thermochromic film and ultrasonic. Certain components of the energy window, such as the electro-conductive components of the energy window, could comprise a cermet. A second energy window may also be included in some embodiments, and may comprise yet another radiofrequency emitting device or another variety of energy emitting device. In some embodiments, energy windows 207 may only be substantially planar, or may take on other cross-sectional shapes that may correspond with a portion of the shape of the shaft, such as arced, stair-step, or other geometric shapes/curvatures. In the depicted embodiment, energy window 207 is adjacent to protrusions 204 and 251a, however other embodiments are contemplated in which an energy window may be positioned elsewhere on the modular TDM shaft 202 or modular TDM tip 201 of the wand, and still be considered adjacent to protrusions 204 or 251a. However, if an energy window was placed on modular TDM shaft 202, such an energy window would not be considered adjacent to protrusions 204 or 251a. It is contemplated that in alternative embodiments, either one or both of the energy windows may be omitted.

The depicted embodiment comprises a plurality of axial protrusions 204 (axially meaning at least substantially parallel to an axis of a corresponding TDM shaft). This embodiment further comprises a plurality of non-axial protrusions, such as 251a along the right side of the tip and a plurality of non-axial protrusions positioned along the left side of the tip. The tip further comprises two non-axial corner or transitional protrusions 254. The tip further comprises a plurality of recessions, such as 253a. One or more of the recessions may further comprise a lysing element, such as 253a & 205.

In the depicted embodiment, modular TDM 200 comprising a modular TDM tip 201 with 3 different shaped lysing elements. Lysing element 205 is concave, lysing element 258.2 is straight as are the other lysing elements on the same side, lysing element 253a is convex, as are the other lysing elements on its same side. Non-axial protrusions 251a and the axial protrusion opposite it across the axial midline point in substantially opposite directions. In the depicted embodiment, the double lines on the lysing elements indicate a bevel that is sharpened. In other embodiments, the bevel is not required to be sharp. In other embodiments the lysing element may be a wire. In some embodiments, all the lysing elements are of the same shape. In some contemplated embodiments, one or more of the lysing elements may differ from the rest in shape and/or other characteristics.

In some embodiments, one or more sensors such as for example sensors 210 and 214 may be positioned on the device. The sensors 210 and 214 may comprise any of the sensors described in the specification herein. Other embodiments may comprise one or more sensors on any other suitable location on the TDM, including but not limited to on the protrusions or otherwise on the tip, and on the shaft. Such sensors may comprise for example thermal sensors, photoelectric sensors, photo optic sensors, and/or cameras, etc. In some embodiments, one or more sensors may be used to monitor the local post passage electrical impedance or thermal conditions that may exist near the distal tip of the shaft or on the tip. Some embodiments may also comprise one or more sensors incorporating MEMS (Micro Electro-Mechanical Systems) technology, such as MEMS gyroscopes, accelerometers, galvanometers, piezoelectrics, mechanical scanning elements, diffractive elements, acousto-optic elements, capacitive sensor arrays and the like. Such sensors may be positioned at any number of locations on the TDM, including within the handle in some embodiments. In some embodiments, sensor 214 may comprise fiberoptic elements. In an embodiment, the sensor can be configured to sense a temperature of tissue adjacent to the apparatus during one or methods described herein. The temperature sensor may alternatively be configured to sense a temperature of one or more fluids adjacent to the apparatus such as for example tissue fluids and/or fluids introduced by the surgeon.

Modular TDM tip 201 may further comprise Internal Tissue Optical Motion Sensor (ITOMSensor) 219. ITOMSensor 219 may comprise one or more lenses and/or windows for emitting EMR and may further comprise one or lenses and/or windows for capturing or receiving reflected EMR.

In order to improve the ability of the TDM to track movement with respect to an internal tissue or region of tissue, such as the fatty undersurface of the skin, some embodiments may comprise an ITOMSensor comprising an electromagnetic emission source and a photodetector device. In, alternative embodiments, the internal tissue optical motion sensor may comprise a non-coherent light source. In, the depicted embodiment, the ITOMSensor comprises a coherent light source. Some such embodiments may comprise a motion detection algorithm configured to extract peaks & nulls for detected speckled light intensity patterns. One example of such an algorithm is disclosed in U.S. Pat. No. 7,876,307 titled ‘Motion Detection Mechanism for Laser Illuminated Optical Mouse Sensor’ which is hereby incorporated by reference in its entirety Other examples of an optical sensor that may be used as an ITOMSensor are disclosed in US Patent Application publication number 2013/0016041 titled ‘High Resolution Mouse’ which is hereby incorporated by reference in its entirety. Other examples an optical sensor that may be used as an ITOMSensor are disclosed in: U.S. Pat. No. 7,791,590B1 titled: ‘Optical Mouse With Uniform Level Detection & U.S. Pat. No. 5,578,813 titled: ‘Freehand Image Scanning Device Which Compensates For Non-Linear Movement’ & U.S. Pat. No. 5,644,139 titled: ‘Navigation For Detecting Movement Of Navigation Sensors Relative To An Object’ & U.S. Pat. No. 5,786,804 titled: ‘Method and System For Tracking Attitude,’ which are hereby incorporated by reference in their entirety. One or more steps, features, elements, or components disclosed in any of the above referenced documents which have been incorporated herein by reference may be combined with any other step disclosed in any of the other steps, features, elements, or components disclosed in any of the other documents disclosed herein incorporated by reference herein as would be apparent to one of ordinary skill in the art.

An ITOMSensor may comprise a source of non-coherent light which may be part of the motion sensor or located elsewhere on the surgical tool for illuminating a tissue at a low angle of incidence, a two dimensional array of photodetectors, each of the photo detectors producing an output in response to light reflected from surface irregularities in the tissue, and circuitry (which may be in the surgical tool or located remotely) to track movement of the housing relative to the work surface by comparing at least some of the photo detector outputs sensed at a first time with at least some of the photo detector outputs sensed at a second time if a particular condition in the photo detector outputs is identified. Motion produces successive frames of translated patterns of pixel information, which may be compared to determine the direction and distance moved.

One method for motion detection that may be used in connection with one or more embodiments disclosed herein may be based on based on the “Peak/Null Motion Detection” algorithm described in the International Patent Application WO_{—}03/049018 which reference is incorporated by reference herein in its entirety.

According to the “Peak/Null Motion Detection” algorithm a distinction is made between edges according to their “direction” which may be referred to as edge direction data. In some embodiments based upon the Peak/Null Motion Detection Algorithm, the ITOMSensor may comprise a coherent light source and a photodetector array. In connection with such embodiments the ITOMSensor may operate by illuminating under a determined gradient using a coherent light source a portion of tissue at a determined flash rate; detecting by means of the photodetector array a reflected speckled light intensity pattern from the illuminated portion of the internal tissue surface for a first flash; detecting a second reflected speckled light intensity pattern of the illuminated portion of the internal tissue surface for a second flash; extracting motion features of two different types from the detected first and second speckled light intensity patterns; determining a measurement of the relative motion between the surgical tool and the illuminated portion of the internal tissue surface based on a comparison of motion features extracted. In some embodiments, before the step of determining the measurement a preliminary step may be performed to modify or adjust the gradient under which the surface is illuminated. In some such implementations, the gradient may be modified or adjusted by adjusting the position of the coherent light source and/or moving an optical lens that may be used to obtain an optical gradient. In some implementations, the step of extracting motion features may comprise comparing light intensity using an offset between adjacent pixels derived from speckled light intensity patterns and extracting motion features using this comparison. In some further implementations, an intensity threshold may be defined without extracting motion features; such an intensity threshold may be adjustable. In other implementations, a plurality of intensity thresholds may be defined. In some embodiments, the method may comprise keeping only pairs of neighboring Peaks and Nulls and ignoring individually occurring Peaks and Nulls.

One or more steps, features, elements, or components disclosed in any of the above referenced documents which have been incorporated herein by reference may be combined with any other step disclosed in any of the other steps, features, elements, or components disclosed in any of the other documents disclosed herein incorporated by reference herein as would be apparent to one of ordinary skill in the art.

In some embodiments, ITOMSensor 219 may comprise for example one or more elements described in the aforementioned incorporation by reference. In alternative embodiments ITOMSensor 219 may be located elsewhere on modular TDM 200 such as for example on shaft 202. In some embodiments, ITOMSensor 219 may be coupled with lysing elements and/or energy window to facilitate controlled delivery of energy to tissues. For example, some embodiments may be configured to deliver a preset amount of energy such that when the device is moved faster a larger amount of energy is delivered per unit time, and when the device is moved more slowly the amount of energy is automatically decreased so as to provide a more uniform or at least semi-uniform (at least within a predetermined range) delivery of energy per unit area. In some embodiments, motion 219 sensor may further or alternatively be coupled with sensor 210 and/or 214 for example, in embodiments comprise a temperature sensor; temperature data may be used along with data from ITOMSensor 219 in order to adjust energy output to energy window 207 and/or lysing elements.

Temperature and impedance values may be tracked on a display screen or directly linked to a microprocessor capable of signaling control electronics to alter the energy delivered to the tip when preset values are approached or exceeded. Typical instrumentation paths are widely known, such as thermal sensing thermistors, and may feed to analog amplifiers which, in turn, feed analog digital converters leading to a microprocessor. In some embodiments, internal or external ultrasound measurements may also be taken during a procedure with the TDM. Sensors that may be useful include thermal sensors, photoelectric or photo optic sensors, cameras, etc. Temperature sensors that may be useful in connection with embodiments disclosed herein may comprise, but are not limited to, resistance temperature sensors, such as carbon resistors, film thermometers, wire-wound thermometers, or coil elements. Some embodiments may comprise thermocouples, pyrometers, or non-contact temperature sensors, such as total radiation or photoelectric sensors.

In some embodiments, one or more electromagnetic delivery elements 215 may be positioned on tip or shaft. Other embodiments may comprise one or more electromagnetic delivery elements on any other suitable location on the TDM, including but not limited to on the protrusions or otherwise on the tip, and on the shaft. Electromagnetic delivery elements that may be useful include: LEDs, LASERs, fiberoptics, filaments, photoelectric materials, infrared emitters, etc. One or more additional conduits 212.3 may be used to transmit data from one or more locations on the modular TD 200 such as for example sensors 210, 214, 216, 219; conduit 212.3 may further be configured to deliver signals and/or energy to modular TD 200. In alternative embodiments separate conduits may be used for delivery of signals and/or energy from conduits used to extract data from modular TD.

In embodiments of tips with at least some non-axial placement of protrusion and or relative recessions, surgeons may implement the use of a fanning motion which may comprise a ‘windshield wiper’ motion.

In the embodiment depicted in FIGS. 2a,b antenna 218 comprises an RFID TAG. In embodiments in which antenna 218 comprises an RFID tag, the RFID tag may comprise a RFID transponder. In other embodiments the RFID tag may comprise a passive tag. It should be understood that although antenna 218 is not depicted in every one of the other figures, any of the embodiments described herein may include one or more such locations. Other embodiments may comprise one or more antennas on any other suitable location on the TDM, including but not limited to on the protrusions or otherwise on the tip, and on the shaft. In some embodiments an RFID transponder or other antenna may comprise a microchip such as a microchip having a rewritable memory. In an embodiment the tag is millimeter sized. In some embodiments a reader generates an alternating electromagnetic field which activates the antenna/RFID transponder and data is sent via frequency modulation. In an embodiment, the position of the antenna/RFID tag is determined by an alternating electromagnetic field in the ultra-high frequency range. The position may be related to a 3 dimensional mapping of the subject. In an embodiment the reader may generate an alternating electromagnetic field. In a further embodiment the alternating electromagnetic field may be in the shortwave (13.56 MHz) or UHF (865-869 MHz) frequency.

In some embodiments, a modular TDM 200 may be attached to a robotic arm. In some embodiments modular TDM tip 201 and portion of modular TDM shaft 202 may be attached to a robotic arm. In some embodiments modular TDM tip 201 and a portion of modular TDM shaft 202 and/or a portion of modular TDM plug 203 may be attached to a robotic arm.

Said modular TDM are not intended to be restricted to symmetry and/or pattern and/or dimension. In other embodiments said modular TDM may be asymmetrical or lacking protrusions and/or lysing elements on one side or another.

One or more electrosurgical conduits may extend from 200. For example, in the embodiment depicted in FIGS. 2a,b conduit 212 may extend to source of electrosurgical energy and may use connector 212.2 to plug into the source. A separate sensor conduit may be used to send and/or receive one or more signals, energy or related data or materials to be used in connection with one or more of the sensors on the device.

In the depicted embodiment, electrosurgical ‘pencil’ comprises an electrosurgical ‘pencil’ shaft 202.1, electrosurgical ‘pencil’ handle 203.1 & rocker switch 203.2 located on or about ‘pencil’ handle 203.1 and/or ‘pencil’ shaft 202.1 which may control the electrosurgical energy such as suitable electrosurgical waveform derived from an electrosurgical generator (not seen in this view) brought into the handle via conduit 211.2. In some embodiments, modular TDM 200 is modular in that it may removable from electrosurgical ‘pencil’ shaft 202.1. More particularly, modular TDM 200 comprises a means for removably coupling the tip with an electrosurgical ‘pencil’ shaft 202.1 at 268. In the depicted embodiment, this coupling means comprises a modular TDM tip plug 203. In some embodiments, modular TDM tip plug 203 may be threaded to facilitate a secure coupling between modular TDM 200 and ‘pencil’ shaft 202.1. However, in other embodiments, the coupling means may comprise a recess configured to receive a plug formed on the shaft. In still other embodiments, the coupling means may comprise a snap-fit coupling, a friction fit coupling, a bayonet clip, etc.

With regards to the term ‘electrosurgical pencil,’ the following is taken from the publication of Bovie Medical “Understanding Electrosurgery” © 2010 written by Genard McCauley page 7: “Monopolar electrosurgery has the means of delivering energy to the tissue through several modalities (modes of operation): pure cut, blended cut, desiccation or pinpoint) and spray (or fulguration). The delivery system of the monopolar electrosurgical generator can be a hand controlled pencil (reusable or disposable) or a foot controlled pencil. A number of accessories can be adapted to the foot control output jack to deliver energy through a number of instruments.

In the depicted embodiment modular TDM tip plug 203 is configured to be received within a corresponding electrosurgical ‘pencil’ shaft recess 269 formed within electrosurgical ‘pencil’ shaft 202.1. In some embodiments, elements within modular TDM tip plug 203 and/or recess 269 may be electrically connected with electrical elements within ‘pencil’ shaft 202.1 and/or ‘pencil’ handle 203.1 which are in turn connected with ‘pencil’ switch 203.2 and/or conduit 211.2. In some embodiments, modular TDM tip plug 203 may be configured to electrically couple modular TDM tip 201 with ‘pencil’ shaft 202.1. In this manner, in embodiments comprising, for example, lysing elements, electricity from a power source may be transmitted through the coupling between modular TDM tip plug 203 and ‘pencil’ recess 269 to allow for energizing the lysing elements.

In some embodiments, modular TDM 200 may be disposable as well, such that a surgeon can place an appropriate tip on the shaft and remove and dispose of the tip after surgery. Alternatively or additionally, a plurality of different tips may be provided, each of which may be disposable, or may be configured for sterilization and re-use, and an appropriate tip may be selected as needed for a particular surgery.

In the depicted embodiment, modular TDM tip 201 comprises a plurality of protrusions 204, some of which are non-axial, and a plurality of recessions that may contain lysing elements 205 positioned therebetween, as described above. In some embodiments, a modular TDM and/or a modular TDM tip comprising only axial protrusions may be swapped for modular TDM tip 201 as desired to suit a particular surgical procedure.

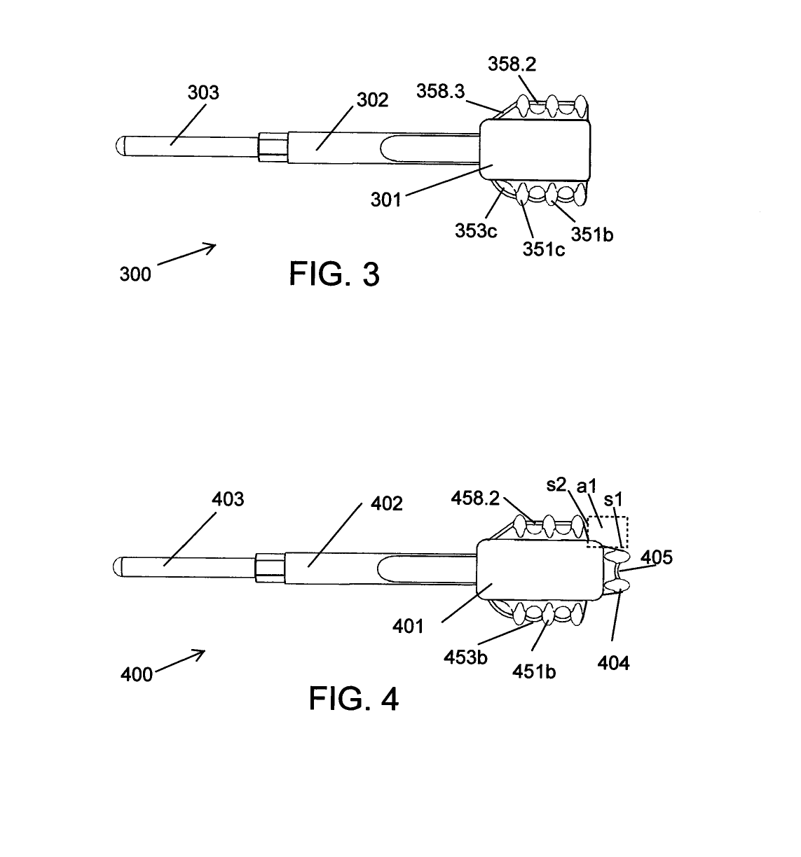

FIG. 3 is an upper plan view illustrating an embodiment of a modular Tissue Dissector (modular TD) 300 comprising a modular TD tip 301, a modular TD shaft 302 and a modular TD plug 303, wherein the tip is lacking in axial protrusions and lysing elements. In the depicted embodiment, protrusions and lysing elements are however oriented in non-axial directions. In other words, protrusions 351b, 351c, & 353c are not oriented in a direction of the axis of modular TD shaft 302. The embodiment depicted in FIG. 3 also lacks in transitional protrusions. Transitional protrusions, axial protrusions, non-axial protrusions, axial lysing elements, non-axial lysing elements and lysing elements of differing geometries are discussed in more detail elsewhere in this application. Embodiments having only non-axial protrusions may be useful in ‘open’ surgical procedures wherein a surgeon may predominately move the TD in a side to side procedure; such embodiments may also be useful where a blunt non-energized probe front is able to separate and/or pass through and/or around a particular type of tissue; such embodiments may also be easier to manufacture from a cost and/or other perspective. Such an embodiment lacking in axial protrusions may be more difficult to move in a forward direction through, for example, more fibrous and/or dense tissues. In the depicted embodiment, non-axial lysing element 358.2 is straight as are the other lysing elements such as 358.3 on the same side; lysing element 353c is convex, as are the other lysing elements on its same side. In other contemplated embodiments, a variety of geometrically shaped lysing elements and/or protrusions may co-exist side by side. For example, in some embodiments, two adjacent protrusions may differ in shape and or size, 2 adjacent lysing elements may differ in length and or shape and/or TD tip 301 may otherwise be nonsymmetrical in one or more views. In some embodiments, the number of protrusions per side may range from 2 to 10. In some embodiments the number of protrusions per side may range from 1 to 99. In some embodiments, the modular TD tip without axial protrusions may measure and/or range in sizes similar to previously discussed embodiments in this application.

In some embodiments, non-axial protrusion such as 351b may extend at an angle of between zero degrees and 30 degrees of a normal to the direction in which the axes of shaft 302 and/or tip 301 point. It is contemplated that it may be desirable for some implementations and/or embodiments to provide non-axial tips extending in a direction or directions falling within this range in order to, for example, allow a surgeon to effectively perform both a to and fro, and a side-to-side (“windshield wiper”) motion using the TD.

FIG. 4 is an upper plan view illustrating an embodiment of a modular Tissue Dissector (modular TD) 400 with a modular TD tip 401, a modular TD shaft 402 and a modular TD plug 403, wherein the tip is lacking in transitional protrusions and transitional lysing elements. Protrusions and lysing elements, however, may be oriented in axial and/or ‘normal’ (perpendicular) directions. In other words, non-axial protrusion 451b is substantially oriented in a ‘normal’ to direction (substantially perpendicular to) the axis of modular TD shaft 402; and axial protrusion 404 axes are substantially parallel to the axes of modular TD shaft 402 and/or modular TD tip 401. Transitional protrusions, axial protrusions, non-axial protrusions, axial lysing elements, non-axial lysing elements and lysing elements of differing geometries are discussed in more detail elsewhere in this application. Embodiments having only non-axial and axial protrusions may be useful in ‘open’ surgical procedures wherein a surgeon may predominately move the TD predominately side to side and/or forward directions without moving in intermediate angles for a particular procedure; such embodiments may also be useful where tissues are relatively soft or tissue planes are more easily maintained and/or well defined. Such embodiments may also be a bit easier to manufacture from a cost and/or other perspective. Such an embodiment lacking in transitional protrusions may be more difficult to move in a directions ranging around 45 degrees from the modular TD shaft axis. In the depicted embodiment, non-axial lysing element 458.2 is straight and opposing side lysing elements, such as 453b, are convex; whereas axially directed lysing element 405 is concave. In other contemplated embodiments, a variety of geometrically shaped lysing elements and/or protrusions may co-exist side by side. For example, in some embodiments, two adjacent protrusions may differ in shape and/or size, 2 adjacent lysing elements may differ in length and/or shape and/or TD tip 401 may otherwise be nonsymmetrical in one or more views. In some embodiments, non-axial protrusion such as 451b may extend at an angle of between zero degrees and 30 degrees of a normal to the direction in which the axes of shaft 402 and/or tip 401 point. It is contemplated that it may be desirable for some implementations and/or embodiments to provide non-axial tips extending in a direction or directions falling within this range in order to, for example, allow a surgeon to effectively perform both a to and fro, and a side-to-side (“windshield wiper”) motion using the TD. In some embodiments, the number of protrusions per side may range from 2 to 10. In some embodiments the number of protrusions per side may range from 1 to 99. In some embodiments, the modular TD tip without axial protrusions may measure and/or range in sizes similar to previously discussed embodiments in this application. In the depicted embodiment, a1 is the area of a rectangle representing a space remaining if there is no transitional protrusion and/or lysing element; s1 represents the side parallel to the axis of the modular TD shaft whereas s2 represents the side perpendicular to s1. In some contemplated embodiments, a1 may contain axial or transitional protrusions pointing at angles within about 30 degrees of s1 and may populate most of the area depicted in a1. In some embodiments, a1 may contain non-axial or transitional protrusions pointing at angles within about 30 degrees of s2 populating most of area a1. In some embodiments, the same options discussed with reference to a1 may similarly be positioned on a space on the opposite side of modular TD tip 401 relative to a1.

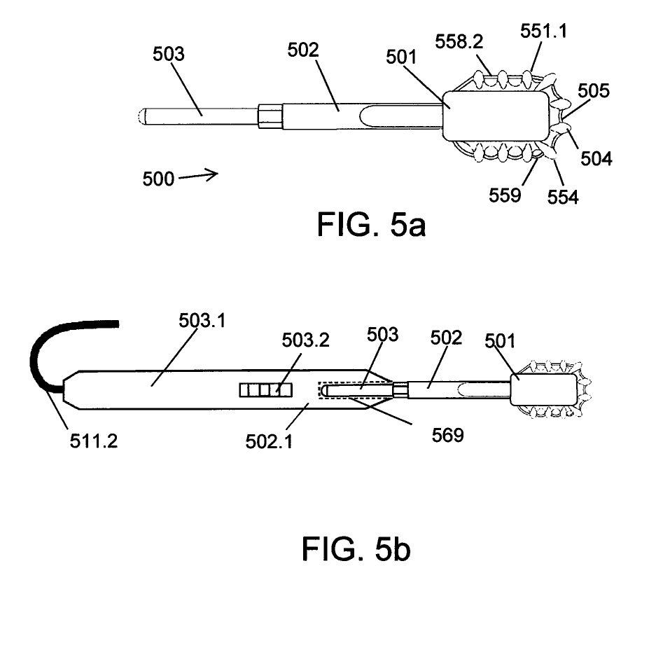

FIG. 5a is an upper plan view illustrating an embodiment of a modular Tissue Dissector (modular TD) 500 comprising a modular TD tip 501, a modular TD shaft 502 and a modular TD plug 503, wherein the tip lacks a means for spot coagulation. Notably, the depicted embodiment lacks a spot coagulating lysing element. The depicted embodiment also comprises axial protrusion 504, transitional protrusion 554 and non-axial protrusion 551.1, non-axial, straight lysing element 558.2, concave axial lysing element 505 and convex lysing element 559. In the depicted embodiment convex lysing element 559 does not protrude substantially relative to other electrosurgically energizable lysing elements; these features may discourage use of the lysing elements for spot coagulation. In the depicted embodiment, convex lysing element 559 may also be somewhat shielded from significant tissue exposure on forward motion to restrict its ability to be forwardly pushed toward a bleeding area to spot coagulate said area; as well an approach from an non-axial motion may be difficult in that the closest non-axial protrusion may also provide some shielding. Transitional protrusions, axial protrusions, non-axial protrusions, axial lysing elements, non-axial lysing elements and lysing elements of differing geometries are discussed in more detail elsewhere in this application. Embodiments lacking protruding spot coagulation lysing elements may be useful in ‘open’ surgical procedures wherein little bleeding is expected; such embodiments may also be a bit easier to manufacture from a cost and/or other perspective. In other contemplated embodiments, a variety of geometrically shaped lysing elements and/or protrusions may co-exist side by side. For example, in some embodiments, two adjacent protrusions may differ in shape and/or size, 2 adjacent lysing elements may differ in length and/or shape and/or TD tip 501 may otherwise be nonsymmetrical in one or more views.

FIG. 5b is an upper plan view of a Modular TD 500 that may be removable from an electrosurgical ‘pencil’ shaft and/or an electrosurgical ‘pencil’ handle 503.1. Rocker switch 503.2 may control the electrosurgical energy such as a suitable waveform derived from an electrosurgical generator (not seen in this view) brought into the handle via conduit 511.2.

In the depicted embodiment modular TDM tip plug 503 is configured to be received within a corresponding electrosurgical ‘pencil’ shaft recess 569 formed within electrosurgical ‘pencil’ shaft 502.1. In some embodiments, elements within modular TDM tip plug 503 and/or electrosurgical ‘pencil’ shaft recess 569 may be electrically connected with electrical elements within ‘pencil’ shaft 502.1 and/or ‘pencil’ handle 503.1 which are in turn connected with ‘pencil’ switch 503.2 and/or conduit 511.2.

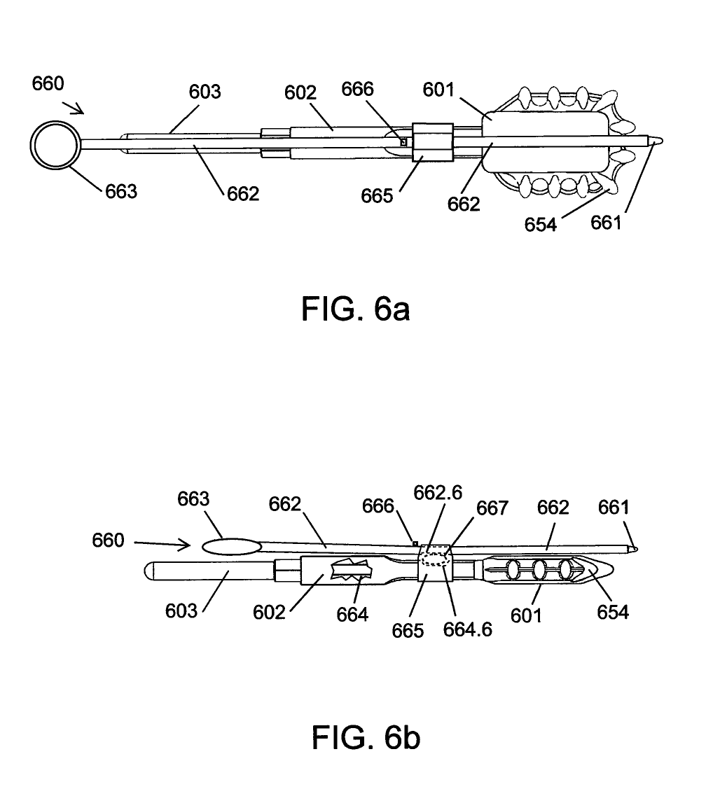

FIG. 6a is an upper plan view of an embodiment of modular TD with a spot coagulator (SC). Modular TD comprises a Modular TD tip 601, a Modular TD shaft 602, a Modular TD plug 603 and spot coagulator 660. In the depicted embodiment, Modular TD tip 601 comprises transitional protrusions 654. Spot coagulator 660 comprises an SC tip 661, SC shaft 662 and SC handle 663. In the depicted embodiment, modular TD shaft 602 may comprise an insulating and/or supportive coating and/or cover overlying a modular TD shaft conductive core. In the depicted embodiment, SC shaft 662 is slidably coupled to the modular TD shaft 602 of the modular TD by SC coupler 665. SC coupler 665 is one example of a means for moveably coupling a SC with a surgical tool. SC coupler 665 may be configured to allow SC shaft 662 to slide relative to SC coupler 665 and modular TD shaft 602. In the depicted embodiment, SC coupler 665 is rigidly affixed to SC shaft 662. In contemplated embodiments, SC coupler 665 may be configured to slide relative to TD shaft 602. In some embodiments, SC coupler 665 may comprise a sterilizable plastic and/or polymer and/or ceramic and/or polytetrafluoroethylene and/or other non-conductive and/or insulating material. In some embodiments, SC coupler 665 may comprise an integral part of modular TD shaft 602. In other embodiments SC coupler 665 may comprise a separate component that may be coupled with modular TD shaft 602 by way of for example form fitting, snap fitting, an adhesive, welding, and the like. One or more passageways in SC coupler 665 may allow the SC shaft 662 to pass therethrough. In some contemplated embodiments, SC coupler 665 may be formed from and/or be an integral part of modular TD shaft 602; in some of these embodiments, SC coupler 665 may comprise the same insulating materials of modular TD shaft 602. In other contemplated embodiments, SC coupler 665 may be formed from and/or be an integral part of SC shaft 662; in some of these embodiments, SC coupler 665 may comprise the same insulating materials of SC shaft 662. The more distal end of the SC is the SC tip 661 and the more proximal (toward the surgeon) end of the SC is SC handle 663. The term handle does not necessarily specify it is for the entire hand of the surgeon as just one finger is possible to control the SC in some embodiments. In the embodiment depicted in FIGS. 6a,b, the SC handle 663 comprises a ring that may be operated by a single finger of a surgeon. In this depicted embodiment the SC tip 661 extends from the SC shaft 662 and is conductive and not insulated along at least a portion of the tip. In some embodiments, the entire tip may be conductive. So as not to have the surgeon set down the TD or TDM, to pick up other instrumentation from the surgical tray to stem bleeding blood vessel(s), it may be beneficial during some surgical procedures to have spot coagulator coagulation capabilities within the same instrument.

FIG. 6b is a depiction of an embodiment revealing a ‘break away’ phantom in the modular TD shaft 602 which shows a portion 664 of the metal center of the shaft as it passes from the TD plug area to TD tip. In some embodiments, the modular TD plug 603 may comprise a continuation of the metal center of modular TD shaft 602; in some embodiments, the diameter may be noncircular and/or irregular on a macroscopic and/or microscopic scale to prevent unwanted rotation. In the depicted embodiment limiting ridge 666 may be present on or about SC shaft 663 to prevent further movement of the SC tip 661 a given distance beyond the modular TD tip 601. In the depicted embodiment the SC tip is restricted to 10 mm protrusion beyond the distalmost portion of modular TD tip 601, which may comprise the end of one of the protrusions on modular TD tip 601. In other contemplated embodiments, a bend in the SC shaft or SC coupler passageway and/or size mismatch and/or tether, etc., may also be used to limit the distance SC tip 661 may protrude beyond the distal end of modular TD tip 601. In other contemplated embodiments, no elements may restrict the working movement range of the SC shaft 662.

FIG. 6b also depicts in phantom 3 elements: internal components of SC coupler 665 including SC shaft conductive opening 662.6 in SC shaft 662, and modular TD shaft conductive opening 664.6 in modular TD shaft 602, and SC coupler conductive spring 667. In the depicted embodiment, SC shaft 662 may comprise one or more non-insulated area(s) (usually on the underside) that may be brought into contact with SC coupler conductive spring 667 which may electrically couple with the modular TD shaft conductive opening 664. For example, some embodiments may comprise a plurality of adjacent conductive rings or other non-insulated areas such that SC tip 661 may be positioned at a plurality of preset distances from modular TD tip. SC coupler conductive spring 667 may comprise a metal and/or other conductive material such as metal-coated/impregnated plastic and/or polymer and may have a variety of shapes. SC shaft conductive opening 662.6 and/or modular TD shaft conductive opening 664.6 may comprise metal portions of their respective shafts lacking overlying insulation in the area of the opening. In contemplated embodiments, SC coupler conductive spring 667 may be an integral part of SC coupler 665 and/or modular TD shaft 602. In the depicted embodiment, the SC coupler conductive spring 667, may comprise a flexible metal having an oval cross section that may be welded and/or affixed to modular TD shaft 602 by any means known in the art.

An implementation using the depicted embodiment may involve pushing distally SC handle 663 which forces SC shaft 662 distally through SC coupler 665 whereupon SC shaft conductive opening 662.6 is brought into direct contact with SC coupler conductive spring 667 which itself may be in direct contact with modular TD shaft conductive opening 664.6. Thus electrosurgical energy such as suitable electrosurgical waveform will be delivered, when the electrosurgical generator is activated, via modular TD plug 603, into modular TD shaft 602, into modular TD shaft conductive opening 664.6 into SC coupler conductive spring 667 into SC shaft conductive opening 662.6 into SC shaft 662 and thereupon to SC tip 661 and then into target tissue. In the depicted embodiment, the SC tip 661 extends from the SC shaft 662 and is conductive and not insulated along at least a portion of the tip. In some embodiments, the entire tip is conductive. In the depicted embodiment SC shaft 662 may comprise stainless steel and may be round in cross-section. In the depicted embodiment, modular TD shaft 602 may comprise an electrically insulating and/or supportive coating and/or cover overlying a conductive material and/or metal axial core. The electrical insulator may comprise, for example, porcelain, ceramics, glass-ceramics, plastics, various halogenated carbon molecules, polytetrafluoroethylene, carbon, graphite, and graphite-fiberglass composites and the like. In some embodiments, the conductive material may comprise: steel, nickel, alloys, palladium, gold, tungsten, silver, copper, platinum and/or any other conductive metal that does not give off toxic residua at operating temperatures. In other contemplated embodiments, the conductive material may comprise cermets and the like. In the depicted embodiment, SC tip 661 is shaped like the frustum of a cone. In some embodiments, SC shaft 662 may be oval, flat, rectangular or geometric in cross-section or substantially flattened. In alternative embodiments, SC tip 661 may be pointed, bullet shaped, or geometric in cross section; more angulate and/or pointed tips may disperse electrical energy more readily and allow greater precision than larger, more rounded tip designs. In the depicted embodiments of the SC shaft 662, the electrical insulator may comprise polytetrafluoroethylene. In alternative embodiments the electrical insulator may comprise polyether etherketone and/or polysulfone and/or another electrically nonconductive polymers (with thermal stability in the operating range) and/or materials that are both electrically non-conductive and of low thermal conductivity. In contemplated embodiments the electrical insulator may comprise, for example, porcelain, ceramics, glass-ceramics, plastics, various halogenated carbon molecules, polytetrafluoroethylene, carbon, graphite, and graphite-fiberglass composites.

Although the depicted embodiment shows a manually deployed SC, other contemplated embodiments may allow deployment to be (including but not limited to): motorized and/or spring activated and/or screw driven and/or ratchet style and/or cog style and/or pneumatic and/or hydraulic, etc. Although, the embodiment depicted in FIG. 6b shows the SC shaft 662 pointing about axially (relative to the modular TD axis) and/or passing about over the middle of the front of the modular TD tip 601, in other contemplated embodiments, SC shaft 662 may point at an angle within about 30 degrees of the axis of the modular TD and/or may pass over any other points on the modular TD tip 601 besides the middle front of the modular TD tip. In other contemplated embodiments, SC shaft 662 may point at angles greater than 30 degrees of the axis of the modular TD. In some embodiments, SC shaft 662 may be flexible and its forward axis redirected upon passing through SC coupler 665; in some embodiments SC coupler may pivot and/or rotate and/or have one or more non-axial passageways for SC shaft 662 that may redirect the axis and/or pointing of the forward part of SC shaft 662. For example, some embodiments may comprise a plurality of SC coupler passageways for SC shaft 662 each pointing in a different direction. In such embodiments one or more such passageways may be axial and one or more may be non-axial. In the depicted embodiment, the SC tip 661 and SC shaft 662 together may measure about, 55 mm in length and about 3 mm in maximal thickness; the SC handle 663 may measure about 20 mm in diameter and about 3 mm in maximal thickness; the overall SC 660 may measure about 75 mm in length. In embodiments wherein an electrical insulation and/or polymeric insulating coating are present on such parts, for example on SC shaft 662 and/or SC handle 663, such insulation may measure about 0.5 mm in thickness. In some embodiments, the insulation thickness may range from about 0.1 mm to 3 mm. Embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses.

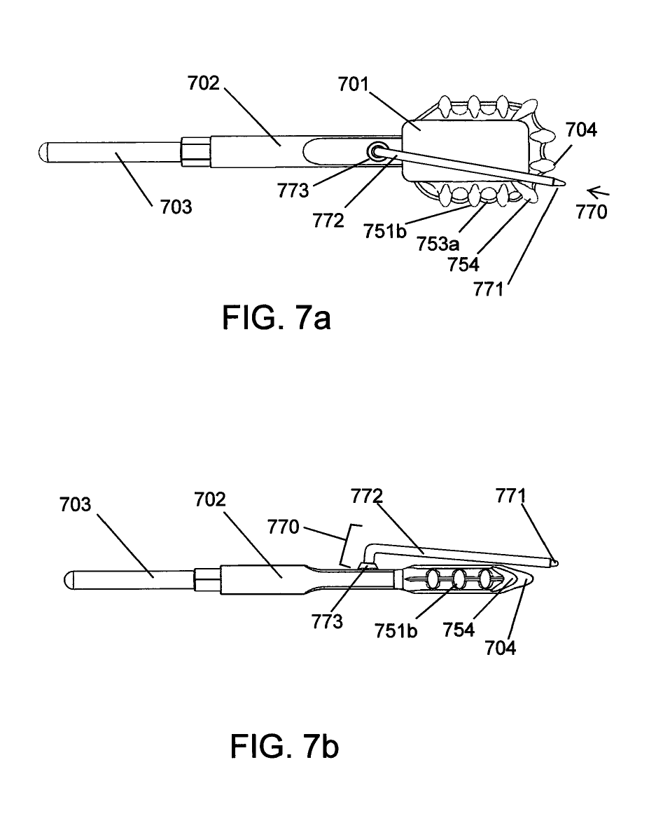

FIG. 7a is an upper plan view of an embodiment of modular TD with a spot coagulator boom. In the depicted embodiment, the Modular TD comprises a Modular TD tip 701, a Modular TD shaft 702, a Modular TD plug 703 and spot coagulator boom (SCB) 770. The depicted embodiment also comprises axial protrusion 704, transitional protrusion 754 and non-axial protrusion 751b, and non-axial convex lysing element 753a. Transitional protrusions, axial protrusions, non-axial protrusions, axial lysing elements, non-axial lysing elements and lysing elements of differing geometries are discussed in more detail elsewhere in this application. In other contemplated embodiments, a variety of geometrically shaped lysing elements and/or protrusions may co-exist side by side. For example, in some embodiments, two adjacent protrusions may differ in shape and/or size, 2 adjacent lysing elements may differ in length and/or shape and/or TD tip 701 may otherwise be nonsymmetrical in one or more views. In the depicted embodiment, modular TD shaft 702 may comprise an electrically insulating and/or supportive coating and/or cover overlying a conductive material and/or metal axial core; the electrical insulator may comprise, for example, porcelain, ceramics, glass-ceramics, plastics, various halogenated carbon molecules, polytetrafluoroethylene, carbon, graphite, and graphite-fiberglass composites and the like. SCB tip 771 shapes may comprise those discussed in other embodiments in this application. Spot coagulator boom 770 comprises a SCB tip 771, SCB shaft 772 and SCB base 773. In the depicted embodiment, the SCB tip 771 extends from the SCB shaft 772 and is conductive and not insulated along at least a portion of the tip. In some embodiments, the entire tip is conductive. In the depicted embodiment, SCB Base 773 permits SCB shaft 772 to pivot about base. In some embodiments, SCB shaft 772 may pivot 360 degrees and in other embodiments, it may be limited to a specific arc, for example some embodiments may be configured to rotate about 180 degrees. Some embodiments may be configured to automatically couple an electric connection at a particular position, for example, some embodiments may be configured such that the spot coagulation boom (SCB) 772 is not electrically connected when the spot coagulator extends backwards toward the modular TD plug 703 but upon reaching a threshold amount of rotation the SCB 770 automatically connects with an electrical source. Alternatively, rotation of the SCB shaft 772 may be independent of the electrical coupling, in such embodiments, a switch may be provided if desired to couple and decouple electrosurgical energy to SCB tip 771. It is contemplated in some embodiments that a snap or hook may be deployed on the modular TD shaft 702 and/or electrosurgical ‘pencil’ that may not only insulate, but provide storage for SCB tip 771 but may clean tip on docking, and may also prevent from inadvertently deploying or coming loose during dissection in close quarters (areas of limited surgical workspace, for example between organs or in a ‘pocket’ of tissues) or more dense tissues.

In the depicted embodiment of FIG. 7b, SCB shaft 772 is angled with respect to the modular TD such that in an operational position as shown in FIG. 7b, tip 771 is angled downward such that SCB tip 771 extends beyond the distal tip of the modular TD and may intersect the axis of the modular TD shaft 702. In such embodiments, when rotated around to a storage position, SCB shaft 772 may extend at a greater angle away from the axis of the modular TDM shaft 702 to provide additional clearance between SCB shaft 772 and modular TDM shaft 702. Such embodiments may be desirable in certain procedures in such that SCB tip 771 may be in a better position for spot coagulation procedures than embodiments in which SCB shaft 772 is parallel to the modular TD shaft 702. Some embodiments may be configured such that SCB shaft 772 is rotatable to discrete number of positions about the base 773 or otherwise with respect to modular TD shaft 702. In alternative embodiments SCB base 773 may be configured to pivot with SCB shaft 772 about modular TD shaft 702. In other contemplated embodiments, SCB 770 may be coupled with other portions of the modular TD such as modular TD tip 701 for example.

In the depicted embodiment, SCB tip 771 and SC shaft 772 together may measure about 30 mm in length and about 2 mm in maximal thickness; SCB base 773 may measure about 4 mm in diameter and about 3 mm in height. In embodiments wherein an electrical insulation and/or polymeric insulating coating is present on such parts, for example SCB shaft 772 and/or SCB base 773 such insulation may measure about 0.5 mm in thickness. In some embodiments, the insulation thickness may range from 0.1 mm to 3 mm. Embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses.

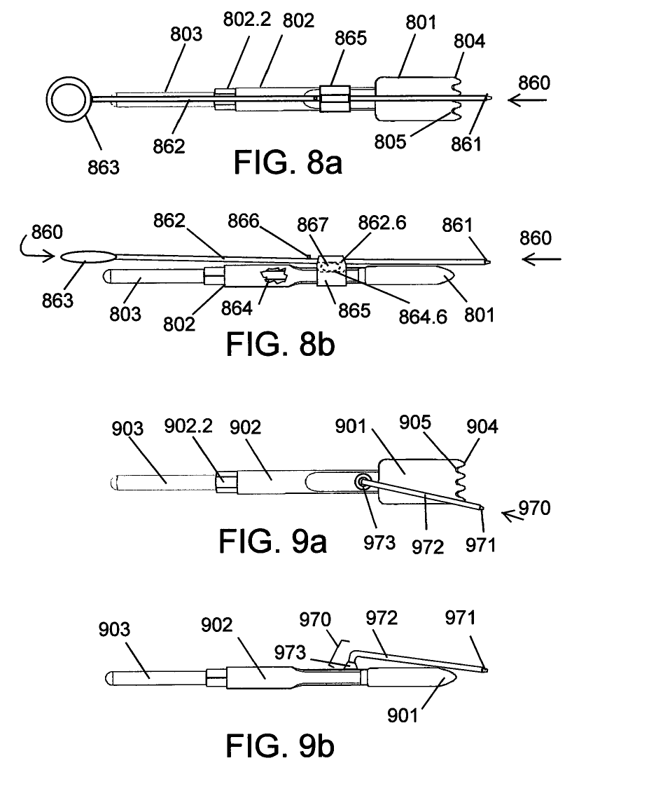

FIGS. 8a,b depict an embodiment that differs from the embodiment depicted in FIGS. 6a,b in that it comprises only axial protrusions 804 and accompanying lysing elements 805. Each of the other elements depicted in FIGS. 8a,b may be identical to the corresponding elements shown in FIGS. 6a,b and are referenced by like numerals (numbers higher by 200). For example, in FIGS. 8a,b: Modular TD comprises a Modular TD tip 801, a Modular TD shaft 802, a Modular TD plug 803 & {spot coagulator (SC) 860 (further comprises: SC tip 861, SC shaft 862 & SC handle 863)}. SC coupler 865 is one example of a means for moveably coupling a SC with a surgical tool. SC coupler 865 may be configured to allow SC shaft 862 to slide relative to SC coupler 865 and modular TD shaft 802. In the depicted embodiment, SC coupler 865 is rigidly affixed to SC shaft 862. In contemplated embodiments, SC coupler 865 may be configured to slide relative to TD shaft 802. In some embodiments, SC coupler 865 may comprise an integral part of modular TD shaft 802. In other embodiments SC coupler 865 may comprise a separate component that may be coupled with modular TD shaft 802 by way of for example form fitting, snap fitting, an adhesive, welding, and the like.

FIG. 8b is a depiction of an embodiment revealing a ‘break away’ in the modular TD shaft 802 which shows a portion 864 of the metal center of the shaft; limiting ridge 866 may be present on or about SC shaft 862. To limit or prevent any rotation of TD plug 803 within a shaft that is a source of electrosurgical energy, the section of SC shaft 802 may comprise SC shaft insert 802.2 that may take any geometric shape and be inserted into the source of electrosurgical energy mating with a complementary shape therein.

FIG. 8b also depicts in phantom 3 elements: internal components of SC coupler 865 including SC shaft conductive opening 862.6 in SC shaft 862, and modular TD shaft conductive opening 864.6 in modular TD shaft 802, and SC coupler conductive spring 867.

Although the depicted embodiment shows a manually deployed SC, other contemplated embodiments may allow deployment to be (including but not limited to): motorized and/or spring activated and/or screw driven and/or ratchet style and/or cog style and/or pneumatic and/or hydraulic, etc. Although, the embodiment depicted in FIG. 8b shows the SC shaft 862 pointing about axially (relative to the modular TD axis) and/or passing about over the middle of the front of the modular TD tip 801, in other contemplated embodiments, SC shaft 862 may point at an angle within about 30 degrees of the axis of the modular TD. In other contemplated embodiments, SC shaft 862 may point at angles greater than 30 degrees of the axis of the modular TD. In some embodiments, SC coupler may pivot and/or rotate and/or have one or more non-axial passageways for SC shaft 862 that may redirect the axis and/or pointing of the forward part of SC shaft 862. For example, some embodiments may comprise a plurality of passageways for SC shaft 862 each pointing in a different direction. In such embodiments one or more such passageways may be axial and one or more may be non-axial. In the depicted embodiment of FIGS. 8a,b, measurements about those of similar parts in FIGS. 6a,b. Embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses.

FIGS. 9a,b depict an embodiment that differs from the embodiment depicted in FIGS. 7a,b in that it comprises only axial protrusions 904 and accompanying lysing elements 905. Each of the other elements depicted in FIGS. 9a,b may be identical to the corresponding elements shown in FIGS. 7a,b and are referenced by like numerals (numbers higher by 200). For example, FIGS. 9a,b: Modular TD comprises a Modular TD tip 901, a Modular TD shaft 902, a Modular TD plug 903 & {spot coagulator boom (SCB) 970 (further comprises: SCB tip 971, SCB shaft 972 & SCB base 973)}. In the depicted embodiment, SCB Base 973 permits SCB shaft 972 to pivot about base. In some embodiments, SCB shaft 972 may pivot 360 degrees and in other embodiments, it may be limited to a specific arc, for example some embodiments may be configured to rotate about 180 degrees. Some embodiments may be configured to automatically couple an electric connection at a particular position, for example, some embodiments may be configured such that the spot coagulation boom (SCB) 972 is not electrically connected when the spot coagulator extends backwards toward the modular TD plug 903 but upon reaching a threshold amount of rotation the SCB 970 automatically connects with an electrical source. Alternatively, rotation of the SCB shaft 972 may be independent of the electrical coupling, in such embodiments, a switch may be provided if desired to couple and decouple electrosurgical energy to SCB tip 971. It is contemplated in some embodiments that a snap or hook may be deployed on the modular TD shaft 902 and/or electrosurgical ‘pencil’ that may not only insulate, but provide storage for SCB tip 971 but may clean tip on docking, and may also prevent from inadvertently deploying or coming loose during dissection in close quarters (areas of limited surgical workspace, for example between organs or in a ‘pocket’ of tissues) or more dense tissues. To limit or prevent any rotation of TD plug 903 within a shaft that is a source of electrosurgical energy, the section of SC shaft 902 may comprise SC shaft insert 902.2 that may take any geometric shape and be inserted into the source of electrosurgical energy mating with a complementary shape therein.

In the depicted embodiment, SCB shaft 972 is angled with respect to the modular TD. Some embodiments may be configured such that SCB shaft 972 is rotatable to discrete number of positions about the base 973 or otherwise with respect to modular TD shaft 902. In alternative embodiments SCB base 973 may be configured to pivot with SCB shaft 972 about modular TD shaft 902. In other contemplated embodiments, SCB 970 may be coupled with other portions of the modular TD such as modular TD tip 901 for example.

In the depicted embodiment of FIGS. 9a,b, measurements about those of similar parts in FIGS. 7a,b, Embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses.

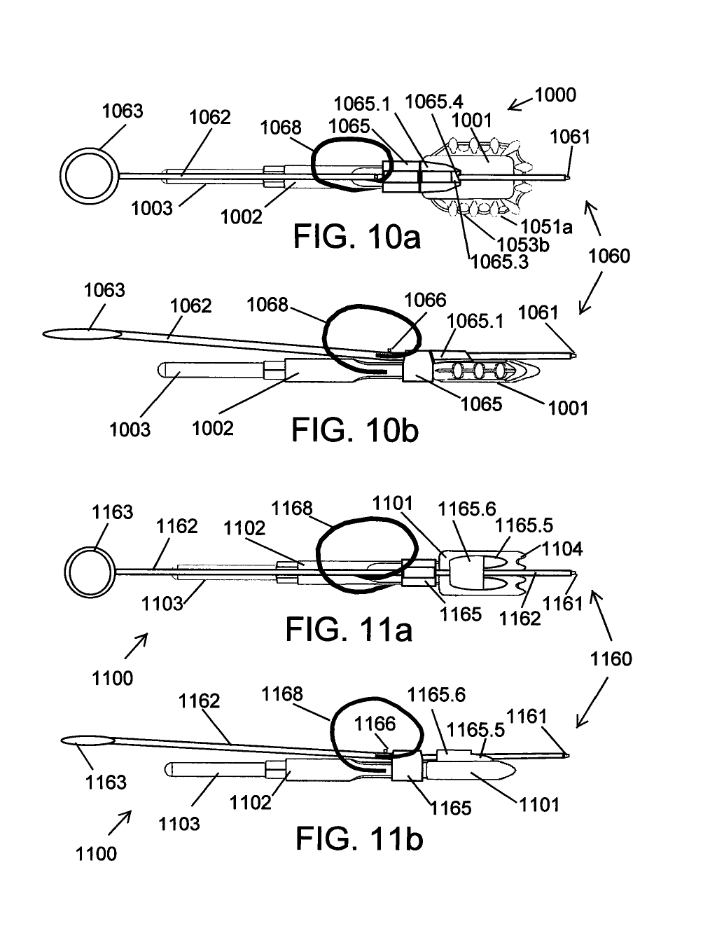

FIG. 10a is an upper plan view of an embodiment of modular TD with a spot coagulator (SC). Modular TD 1000 comprises a Modular TD tip 1001, a Modular TD shaft 1002, a Modular TD plug 1003 & spot coagulator 1060. In the depicted embodiment, Modular TD tip 1001 comprises non-axial protrusions 1051a and non-axial lysing segment 1053b. Spot coagulator 1060 comprises an SC tip 1061, SC shaft 1062, SC handle 1063 & SC connecting conductor cable 1068. In contemplated embodiments, connecting conductor cable may comprise one or more wires, conductive strips, etc. In contemplated embodiments, modular TD may comprise axial protrusions & lack non-axial or transitional protrusions. In the depicted embodiment, modular TD shaft 1002 may comprise an insulating and/or supportive coating and/or cover overlying a conductive material and/or metal axial core. In the depicted embodiment SC shaft 1062 is slidably coupled to the modular TD shaft 1002 of the modular TD by SC coupler 1065. SC coupler 1065 is one example of a means for moveably coupling a SC with a surgical tool. SC coupler 1065 may be configured to allow SC shaft 1062 to slide relative to SC coupler 1065 and modular TD shaft 1002. Alternatively SC coupler 1065 may be rigidly affixed to SC shaft 1062 in which case SC coupler 1065 may be configured to slide relative to TD shaft 1002. In some embodiments, SC coupler 1065 may comprise a sterilizable plastic and/or polymer and/or ceramic and/or polytetrafluoroethylene and/or other non-conductive and/or insulating material. In some embodiments, SC coupler 1065 may comprise an integral part of modular TD shaft 1002. In other embodiments SC coupler 1065 may comprise a separate component that may be coupled with modular TD shaft 1002 by way of for example form fitting, snap fitting, an adhesive, welding, and the like.

One or more passageways in SC coupler 1065 may allow the SC shaft 1062 to pass therethrough. In some contemplated embodiments, SC coupler 1065 may be formed from and/or be an integral part of modular TD shaft 1002; in some of these embodiments, SC coupler 1065 may comprise the same insulating materials of modular TD shaft 1002. In other contemplated embodiments, SC coupler 1065 may be formed from and/or be an integral part of SC shaft 1062; in some of these embodiments, SC coupler 1065 may comprise the same insulating materials of SC shaft 1062. The more distal end of the SC is the SC tip 1061 and the more proximal (toward the surgeon) end of the SC is SC handle 1063. The term handle does not necessarily specify it is for the entire hand of the surgeon as just one finger is possible to control the SC in some embodiments. In the embodiment depicted in FIGS. 10a,b, the SC handle 1063 comprises a ring that may be operated by a single finger of a surgeon. In this depicted embodiment the SC tip 1061 extends from the SC shaft 1062 and is conductive and not insulated along at least a portion of the tip. In some embodiments, the entire tip may be conductive.

FIG. 10b is a side view of a modular TD 1000 & SC 1060 comprising connecting conductor cable 1068. In some embodiments, the modular TD plug 1003 may comprise a continuation of the metal center of modular TD shaft 1002; in some embodiments, the diameter may be noncircular and/or irregular on a macroscopic and/or microscopic scale to prevent unwanted rotation. In the depicted embodiment limiting ridge 1066 may be present on or about SC shaft 1063 to prevent further movement of the SC tip 1061 a given distance beyond the modular TD tip 1001. In the depicted embodiment the SC tip is restricted to 10 mm protrusion beyond the distalmost portion of modular TD tip 1001, which may comprise the end of one of the protrusions on modular TD tip 1001. In the depicted embodiment, connecting conductor cable 1068 may also restrict movement of SC tip 1061. In other contemplated embodiments, a bend in the SC shaft or SC coupler passageway and/or size mismatch and/or tether, etc., may also be used to limit the distance SC tip 1061 may protrude beyond the distal end of modular TD tip 1001. In other contemplated embodiments, no elements may restrict the working movement range of the SC shaft 1062.

FIG. 10b also depicts the attachment of SC connecting conductor cable 1068 to SC shaft 1062 and modular TD shaft 1003 and may conduct electrosurgical energy therebetween. In the depicted embodiment, SC connecting conductor cable 1068 may comprise one or more non-insulated area(s) (usually on the ends) that may be brought into electrical contact with SC shaft 1062 and TD shaft 1003. In some embodiments, SC connecting conductor cable 1068 is rigidly affixed at the contact points by such methods as welding, soldering, gluing, tying, wrapping, twisting and by any other means known in the art. In the depicted embodiment, insulation covers the contact points; electrical insulation may prevent ionic body fluids from transmitting electrosurgical energy to an undesirable location on the patient. In the depicted embodiment, the insulation is polytetrafluoroethylene as it may have a favorable permittivity. In some embodiments, the electrical insulation may comprise, for example, various halogenated carbon molecules, polyether etherketone and/or polysulfone and/or another electrically nonconductive polymers (with thermal stability in the operating range) and/or materials that are both electrically non-conductive and of low thermal conductivity. In contemplated embodiments, the electrical insulator may comprise, carbon, graphite, and graphite-fiberglass composites and the like. SC connecting conductor cable 1068 may comprise a metal and/or other conductive material such as metal-coated/impregnated plastic and/or polymer and may have a variety of shapes.

Some embodiments may further comprise a SC sheath 1065.1 wherein at least a portion of SC may be positioned in the storage position. In some embodiments, SC sheath may extend from modular TD tip 1001. In other embodiments SC sheath 1065.1 may extend from SC coupler 1065. SC sheath may comprise an insulating material and may be configured to completely cover SC tip 1061 in the storage position. SC sheath 1065.1 may, in some embodiments, comprise a transparent material to allow a surgeon to visualize the position of SC tip 1061 within the sheath. Some embodiments may be further configured to clean SC tip 1061 upon withdrawal into SC sheath 1065.1 and/or upon protrusion through SC sheath 1065.1. For example, in the depicted embodiment, SC sheath 1065.1 may also comprise an SC gate 1065.3, which may be hinged from a polymer to allow SC opening 1065.4 to move which may help remove debris from SC tip 1061 on passage into and/or out of SC opening 1065.4. In some embodiments, SC opening 1065.4 may be formed within SC gate 1065.3 and may be configured such that upon advancing and/or retracting SC tip 1061 SC gate 1065.3 pivots slightly to provide an interface with SC tip 1061 that scrapes or otherwise removes debris from SC tip 1061 upon advancing and/or retracting SC tip 1061 thru SC opening 1065.4.

In some embodiments, one or more passageways may be formed in one or more locations on a tip or other area of a TDM to allow for an SC to be repositioned by a surgeon at a plurality of preconfigured positions.

In the depicted embodiment, the SC tip 1061 extends from the SC shaft 1062 and is conductive and not insulated along at least a portion of the tip. In some embodiments, the entire tip is conductive. In the depicted embodiment SC shaft 1062 may comprise stainless steel and may be round in cross-section. In the depicted embodiment, SC shaft 1063 may comprise an electrically insulating and/or supportive coating and/or cover overlying a conductive material and/or metal axial core. In some embodiments, the conductive material may comprise: steel, nickel, alloys, palladium, gold, tungsten, silver, copper, platinum and/or any other conductive metal that does not give off toxic residua at operating temperatures. In other contemplated embodiments, the conductive material may comprise cermets and the like. In some embodiments, the electrical insulator may comprise, for example, porcelain, ceramics, glass-ceramics, plastics, various halogenated carbon molecules, polytetrafluoroethylene, carbon, graphite, and graphite-fiberglass composites and the like. In alternative embodiments the electrical insulator may comprise polyether etherketone and/or polysulfone and/or another electrically nonconductive polymers (with thermal stability in the operating range) and/or materials that are both electrically non-conductive and of low thermal conductivity. In the depicted embodiment, SC tip 1061 is shaped like the frustum of a cone. In some embodiments, SC shaft 1062 may be oval, flat, rectangular or geometric in cross-section or substantially flattened. In alternative embodiments, SC tip 1061 may be pointed, bullet shaped, or geometric in cross section; more angulate and/or pointed tips may disperse electrical energy more readily and allow greater precision than larger, more rounded tip designs. In the depicted embodiments of the SC shaft 1062, the electrical insulator may comprise polytetrafluoroethylene. In alternative embodiments the electrical insulator may comprise polyether etherketone and/or polysulfone and/or another electrically nonconductive polymers (with thermal stability in the operating range) and/or materials that are both electrically non-conductive and of low thermal conductivity. In contemplated embodiments the electrical insulator may comprise, for example, porcelain, ceramics, glass-ceramics, plastics, various halogenated carbon molecules, polytetrafluoroethylene, carbon, graphite, and graphite-fiberglass composites.

Although the depicted embodiment shows a manually deployed SC, other contemplated embodiments may allow deployment to be (including but not limited to): motorized and/or spring activated and/or screw driven and/or ratchet style and/or cog style and/or pneumatic and/or hydraulic, etc. Although, the embodiment depicted in FIG. 10b shows the SC shaft 1062 pointing about axially (relative to the modular TD axis) and/or passing about over the middle of the front of the modular TD tip 1001, in other contemplated embodiments, SC shaft 1062 may point at an angle within about 30 degrees of the axis of the modular TD and/or may pass over any other points on the modular TD tip 1001 besides the middle front of the modular TD tip. In other contemplated embodiments, SC shaft 1062 may point at angles greater than 30 degrees of the axis of the modular TD. In some embodiments, SC shaft 1062 may be flexible and its forward axis redirected upon passing through SC coupler 1065; in some embodiments SC coupler may pivot and/or rotate and/or have one or more non-axial passageways for SC shaft 1062 that may redirect the axis and/or pointing of the forward part of SC shaft 1062. For example, some embodiments may comprise a plurality of passageways for SC shaft 1062 each pointing in a different direction. In such embodiments one or more such passageways may be axial and one or more may be non-axial. In the depicted embodiment of FIGS. 10a,b, measurements about those of similar parts in FIGS. 6a,b, Embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses.

FIGS. 11a,b depict an embodiment that differs from the embodiment depicted in FIGS. 10a,b in that it comprises only axial protrusions 1104 and accompanying lysing elements & also differs in that SC sheath has been replaced by SC shield 1165.5 (SC shield may also further comprise SC shield cover 1165.6. Each of the other elements depicted in FIGS. 11a,b may be identical to the corresponding elements shown in FIGS. 10a,b and are referenced by like numerals (numbers higher by 100). For example, in FIGS. 11a,b: Modular TD comprises a Modular TD tip 1101, a Modular TD shaft 1102, a Modular TD plug 1103 & {spot coagulator (SC) 1160 (further comprises: SC tip 1161, SC shaft 1162 & SC handle 1163)}. SC coupler 1165 is one example of a means for moveably coupling a SC with a surgical tool. SC coupler 1165 may be configured to allow SC shaft 1162 to slide relative to SC coupler 1165 and modular TD shaft 1102. In the depicted embodiment, SC coupler 1165 is rigidly affixed to SC shaft 1162. In contemplated embodiments, SC coupler 1165 may be configured to slide relative to TD shaft 1102. In some embodiments, SC coupler 1165 may comprise an integral part of modular TD shaft 1102. In other embodiments SC coupler 1165 may comprise a separate component that may be coupled with modular TD shaft 1102 by way of for example form fitting, snap fitting, an adhesive, welding, and the like.

FIG. 11b is a side view of a modular TD 1100 & SC 1160 comprising connecting conductor cable 1168. In some embodiments, the modular TD plug 1103 may comprise a continuation of the metal center of modular TD shaft 1102. In the depicted embodiment, SC comprises SC limiting ridge 1166, SC connecting conductor cable 1168, and SC shield 1165.5 (wherein at least a portion of SC may be positioned in the storage position). In the depicted embodiment, SC shield 1165.5 comprises two non-conductive polymeric ridges affixed to TD tip 1101 by adhesive; SC shield 1165.5 further comprises SC shield cover 1165.6. In some embodiments, affixed SC shields 1165.5 may comprise electrical insulators including but not limited to polymeric insulators. In some embodiments, SC shields may be a direct extension and thus composition of a modular TD tip 1101. SC shield cover 1165.6 may, in some embodiments, comprise a transparent material to allow a surgeon to visualize the position of SC tip 1161 within the sheath.

In the depicted embodiment of FIGS. 11a,b, measurements about those of similar parts in FIGS. 10a,b, Embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses.

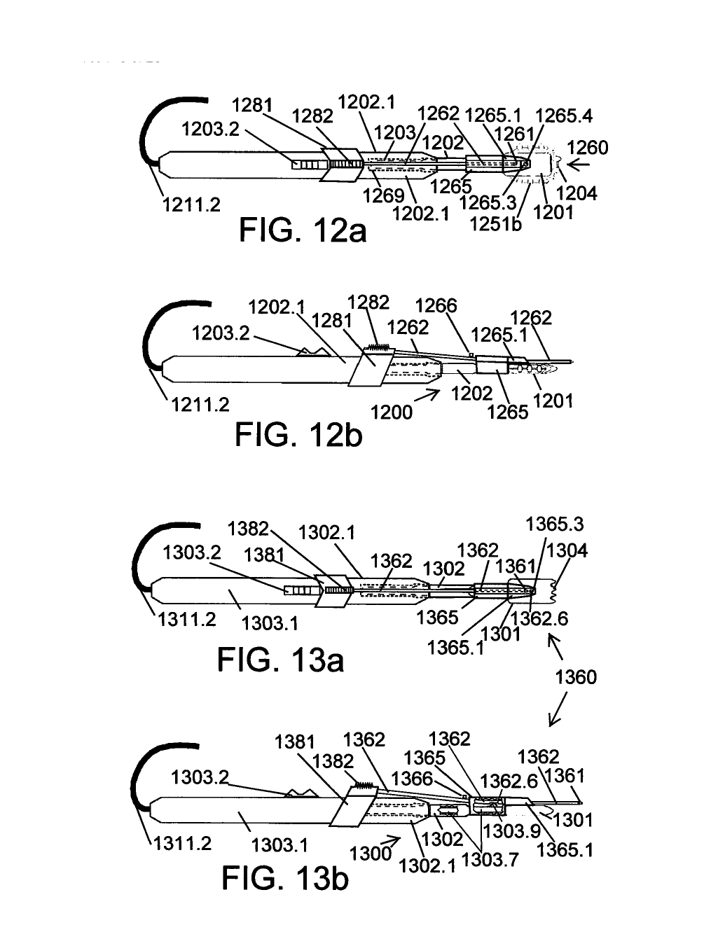

FIGS. 12a,b depict an embodiment comprising an axially advanceable SC 1260. SC comprises SC shaft 1262, SC tip 1261, SC toggle 1281 and/or SC sheath 1265.1. SC shaft may be coupled with modular TD by way of coupler 1265. In this embodiment, toggle 1281 is configured both to advance the SC and to connect the SC with an electrical source at a particular point in its advancement. In contemplated embodiments, separate components may be used to advance the SC and to couple the SC with an electrical source. In the depicted embodiment, SC toggle comprises a sleeve. The modular TD 1200 depicted in this embodiment comprises a Modular TD tip 1201, Modular TD shaft 1202, & Modular TD plug 1203′.

In some embodiments, the spot coagulator toggle may be configured to move at least a portion of the spot coagulator with respect to a portion of a surgical device, such as a tip comprising protrusions and lysing segments, between a plurality of operational positions and a storage position, or between at least one operational position and at least one storage position.

In some embodiments, a first conductive element and a second conductive element may be provided. The first conductive element may be configured to electrically couple to the second conductive element in an operational configuration to allow the spot coagulator tip to deliver energy for coagulating a blood vessel. The first conductive element may be electrically insulated from the second conductive element in a storage configuration to prevent the spot coagulator tip from delivering energy.

In some embodiments, the spot coagulator may be configured to provide for a plurality of operational positions such that at least one conductive element of the spot coagulator couples with at least one other conductive element such that the spot coagulator can deliver energy for coagulating a blood vessel at any of a plurality of operational positions.

In some embodiments, a plurality of conductive elements may be provided and each of the conductive elements may correspond to one of the plurality of operational positions. For example, at least one of a first and a second conductive element may comprise an insulator and a plurality of conducting sections. Each of the conducting sections may comprise an opening in the insulator, such as a bare portion to expose the underlying conductor, to allow the conducting sections to make contact with another conductive element. Each of the conducting sections may correspond to one of the plurality of operational positions such that electrical energy may be deliver to a spot coagulator tip at each, and only at each, of the operational positions.

In some embodiments, the spot coagulator tip may be axially movable with respect to a shaft of a TDM such that the spot coagulator can be advanced to one or more operational configurations and retracted to one or more storage configurations.

The modular TD 1200 depicted in this embodiment has non-axial protrusions. The depicted embodiment also comprises axial protrusions, such as 1204, and non-axial protrusions such as 1251b including transitional protrusions. Transitional protrusions, axial protrusions, non-axial protrusions, axial lysing elements, non-axial lysing elements and lysing elements of differing geometries are discussed in more detail elsewhere in this application. Modular TD 1200 is modular in that it is removable from an electrosurgical ‘pencil’ shaft 1202.1 and/or an electrosurgical ‘pencil’ handle. Rocker switch 1203.2 may control the electrosurgical energy such as a suitable waveform derived from an electrosurgical generator (not seen in this view) brought into the handle via conduit 1211.2. In the depicted embodiment modular TDM tip plug 1203 is configured to be received within a corresponding electrosurgical ‘pencil’ shaft recess 1269 formed within electrosurgical ‘pencil’ shaft 1202.1. In some embodiments, elements within modular TDM tip plug 1203 and/or electrosurgical ‘pencil’ shaft recess 1269 may be electrically connected with electrical elements within ‘pencil’ shaft 1202.1 and/or ‘pencil’ handle which are in turn connected with ‘pencil’ switch 1203.2 and/or conduit 1211.2.

So as not to have the surgeon set down the TD or TDM, to pick up other instrumentation from the surgical tray to stem bleeding blood vessel(s), it may be beneficial during some surgical procedures to have spot coagulator coagulation capabilities within the same instrument.

In the depicted embodiment, modular TD shaft 1202 may comprise an electrically insulating and/or supportive coating and/or cover overlying a conductive material and/or metal axial core. In some embodiments, the conductive material may comprise: steel, nickel, alloys, palladium, gold, tungsten, silver, copper, platinum and/or any other conductive metal that does not give off toxic residua at operating temperatures. In other contemplated embodiments, the conductive material may comprise cermets and the like. In some embodiments, the electrical insulator may comprise, for example, porcelain, ceramics, glass-ceramics, plastics, various halogenated carbon molecules, polytetrafluoroethylene, carbon, graphite, and graphite-fiberglass composites and the like. In alternative embodiments the electrical insulator may comprise polyether etherketone and/or polysulfone and/or another electrically nonconductive polymers (with thermal stability in the operating range) and/or materials that are both electrically non-conductive and of low thermal conductivity. SC tip 1261 shapes may comprise those discussed in other tip embodiments in this application. In the depicted embodiment SC shaft 1262 is slidably coupled to the modular TD shaft 1202 of the modular TD by SC coupler 1265. SC coupler 1265 is one example of a means for moveably coupling a SC with a surgical tool. SC coupler 1265 may be configured to allow SC shaft 1262 to slide relative to SC coupler 1265 and modular TD shaft 1202. In the depicted embodiment, SC coupler 1265 is rigidly affixed to TD shaft 1202. In contemplated embodiments, SC coupler 1265 may be configured to slide relative to TD shaft 1202. In such embodiments, SC coupler 1265 may be rigidly affixed to SC shaft 1262. In some embodiments, SC coupler 1265 may comprise a sterilizable plastic and/or polymer and/or ceramic and/or polytetrafluoroethylene and/or other non-conductive and/or insulating material. In some embodiments, SC coupler 1265 may comprise an integral part of modular TD shaft 1202. In contemplated embodiments, SC coupler 1265 may comprise an integral part of modular TD tip 1201. In other embodiments SC coupler 1265 may comprise a separate component that may be coupled with modular TD tip 1201 by way of for example form fitting, snap fitting, an adhesive, welding, and the like. One or more passageways in SC coupler 1265 and/or SC sheath 1265.1 may allow the SC shaft 1262 to pass therethrough. In some contemplated embodiments, SC coupler 1265 may be formed from and/or be an integral part of modular TD shaft 1202; in some of these embodiments, SC coupler 1265 may comprise the same insulating materials of modular TD shaft 1202. In other contemplated embodiments, SC coupler 1265 may be formed from and/or be an integral part of SC shaft 1262; in some of these embodiments, SC coupler 1265 may comprise the same insulating materials of SC shaft 1262. Some contemplated embodiments may be lacking in SC sheath 1265.1.

Coupler 1265 may be configured to allow SC shaft 1262 to be advanced or retracted axially therethrough. The SC depicted in these figures may be coupled with electrosurgical ‘pencil’ shaft 1202.1 by way of modular TD plug 1203. SC toggle 1281 may be slidably coupled along ‘pencil’ shaft 1202.1 such that SC tip 1261 may be advanced axially to an operational position and retracted axially to a storage position. SC tip 1261 may retract into SC sheath 1265.1 via SC opening 1265.4. In the depicted embodiment, SC sheath may also comprise SC gate 1265.3 which may be hinged from a polymer to allow SC opening 1265.4 to move which may help remove debris from SC tip 1261 on passage into and/or out of SC opening 1265.4. In some embodiments, SC opening 1265.4 may be formed within SC gate 1265.3 and may be configured such that upon advancing and/or retracting SC tip 1261 SC gate 1265.3 pivots slightly to provide an interface with SC tip 1261 that scrapes or otherwise removes debris from SC tip 1261 upon advancing and/or retracting SC tip 1261 thru SC opening 1265.4. Passage of SC shaft through SC opening may be facilitated by such insulations as polytetrafluoroethylene. In some embodiments, the edges of SC opening 1265.4 may be serrated and/or irregularly edged and/or possess bristles and/or other texture that may facilitate dislodgement of electrocoagulation char and/or carbon to clean SC tip 1261. Some contemplated embodiments lack SC gate 1265.3 and have only opening 1265.4. SC shaft 1262 may be limited in excursion by limiting ridge 1266. In the depicted embodiment limiting ridge 1266 may comprise a ridge of SC shaft 1262 insulation that may not fit through SC coupler passageway. SC toggle 1281 may comprise grip 1282 to facilitate advancement and retraction of SC 1260. Some embodiments may further comprise a SC sheath 1265.1 wherein at least a portion of SC may be positioned in the storage position. In some embodiments SC sheath may extend from modular TD tip 1201. In other embodiments SC sheath 1265.1 may extend from SC coupler 1265. SC sheath may comprise an insulating material and may be configured to completely cover SC tip 1261 in the storage position. SC sheath 1265.1 may, in some embodiments, comprise a transparent material to allow a surgeon to visualize the position of SC tip 1261 within the sheath. Some embodiments may be further configured to clean SC tip 1261 upon withdrawal into SC sheath 1265.1 and/or upon protrusion through SC sheath 1265.1. In some contemplated embodiments SC sheath 1265.1 may be absent. SC toggle 1281 may, in some embodiments, comprise a transparent material to allow a surgeon to visualize the position of the front of the electrosurgical ‘pencil’ within the SC toggle. SC toggle 1281 may comprise, in cross section, a full or complete ring. Other embodiments may comprise, in cross section, a split ring. Such rings may be configured to accommodate ‘pencil’ shafts of varying cross sectional geometries and/or diameters and/or lengths. In the depicted embodiment, SC toggle 1281 is attached to the screw-threaded proximal end of conductive metal core of SC shaft 1262; insulation may be removed in the threaded area of the SC shaft to improve the fit. In other contemplated embodiments, SC toggle 1281 may be attached to SC shaft 1262 by way of for example form fitting, snap fitting, an adhesive, welding, and the like. In some contemplated embodiments, SC toggle 1281 may be a continuation and/or comprise the insulation of SC shaft 1262. SC 1260 may be electrically coupled with modular TD 1200 by an SC connecting contact and/or an SC shaft conductive opening (for example, as shown in FIGS. 13 at 1303.9 & 1362.6). The SC connecting contact may be configured to accommodate axial movement of SC shaft 1262 between the operational and storage positions. For example, some embodiments may comprise a plurality of adjacent conductive rings or other non-insulated areas such that SC tip 1261 may be positioned at a plurality of preset distances from modular TD tip. In some contemplated embodiments, toggle 1281 may be a part of modular TD shaft 1202 (and does not move relative to it) instead SC shaft 1262 may move through toggle 1281 and may be controlled by a thumb switch.

In the depicted embodiment, the SC tip 1261 extends from the SC shaft 1262 and is conductive and not insulated along at least a portion of the tip. In some embodiments, the entire tip is conductive. In the depicted embodiment SC shaft 1262 may comprise stainless steel and may be round in cross-section. In the depicted embodiment, SC shaft 1262 may comprise an electrically insulating and/or supportive coating and/or cover overlying a conductive material and/or metal axial core. In some embodiments, the conductive material may comprise: steel, nickel, alloys, palladium, gold, tungsten, silver, copper, platinum and/or any other conductive metal that does not give off toxic residua at operating temperatures. In other contemplated embodiments, the conductive material may comprise cermets and the like. In some embodiments, the electrical insulator may comprise, for example, porcelain, ceramics, glass-ceramics, plastics, various halogenated carbon molecules, polytetrafluoroethylene, carbon, graphite, and graphite-fiberglass composites and the like. In alternative embodiments the electrical insulator may comprise polyether etherketone and/or polysulfone and/or another electrically nonconductive polymers (with thermal stability in the operating range) and/or materials that are both electrically non-conductive and of low thermal conductivity. In the depicted embodiment, SC tip 1261 is shaped like the frustum of a cone. In some embodiments, SC shaft 1262 may be oval, flat, rectangular or geometric in cross-section or substantially flattened. In alternative embodiments, SC tip 1261 may be pointed, bullet shaped, or geometric in cross section; more angulate and/or pointed tips may disperse electrical energy more readily and allow greater precision than larger, more rounded tip designs.

Although the depicted embodiment shows a manually deployed SC, other contemplated embodiments may allow deployment to be (including but not limited to): motorized and/or spring activated and/or screw driven and/or ratchet style and/or cog style and/or pneumatic and/or hydraulic, etc. Although, the embodiment depicted in FIG. 12a shows the SC shaft 1262 pointing about axially (relative to the modular TD axis) and/or passing about over the middle of the front of the modular TD tip 1201, in other contemplated embodiments, SC shaft 1262 may point at an angle within about 30 degrees of the axis of the modular TD and/or may pass over any other points on the modular TD tip 1201 besides the middle front of the modular TD tip. In other contemplated embodiments, SC shaft 1262 may point at angles greater than 30 degrees of the axis of the modular TD. In some embodiments, SC shaft 1262 may be flexible and its forward axis redirected upon passing through SC coupler 1265; in some embodiments SC coupler may pivot and/or rotate and/or have one or more non-axial passageways for SC shaft 1262 that may redirect the axis and/or pointing of the forward part of SC shaft 1262. For example, some embodiments may comprise a plurality of SC coupler and/or SC sheath passageways for SC shaft 1262 each pointing in a different direction. In such embodiments one or more such passageways may be axial and one or more may be non-axial. In other contemplated embodiments, SC coupler 1265 and/or SC sheath 1265.1 may have one or more pathways that may lead to SC shaft exiting at a similar range of angles from the axis of the modular TD.

In other contemplated embodiments, a variety of geometrically shaped lysing elements and/or protrusions may co-exist side by side. For example, in some embodiments, two adjacent protrusions may differ in shape and/or size, 2 adjacent lysing elements may differ in length and/or shape and/or TD tip 1201 may otherwise be nonsymmetrical in one or more views.

In the depicted embodiment, the SC tip 1261 and SC shaft 1262 together may measure about, 55 mm in length and about 3 mm in maximal thickness; the SC toggle 1281 may measure about 15 mm in diameter and about 1 mm in maximal thickness; the overall SC 1260 may measure about 75 mm in length. In embodiments wherein an electrical insulation and/or polymeric insulating coating may be present on such parts, for example on SC shaft 1262 such insulation may measure about 0.5 mm in thickness. In some embodiments, the insulation thickness may range from about 0.1 mm to 3 mm. Embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses.

An implementation using the depicted embodiment may involve the surgeon pushing distally SC toggle 1281 which forces SC shaft 1262 distally through SC coupler 1265 whereupon SC shaft conductive opening is brought into direct contact with SC connecting contact which itself is in direct or indirect contact with the conductive portion of modular TD shaft. Thus electrosurgical energy such as a suitable waveform will be delivered, when the electrosurgical generator is activated, via modular TD plug 1203, into modular TD shaft 1202, into SC shaft 1262 and thereupon to SC tip 1261 and then into target tissue.

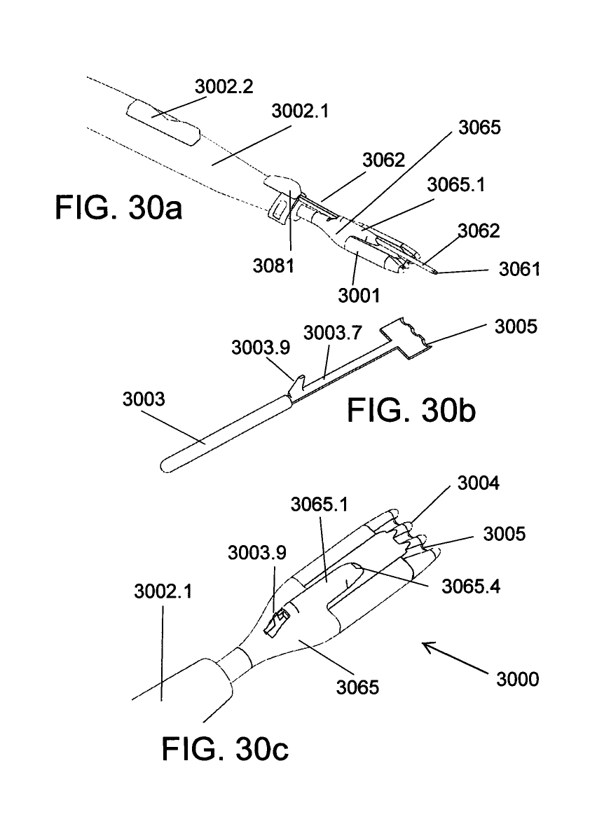

The embodiment depicted in FIGS. 12a and 12b may, in some embodiments, further comprise a protruding electrically-conductive element positioned on an electro-conductive portion of Modular TD shaft 1202 that is configured to electrically couple with a corresponding conductive element positioned on SC shaft 1262. For example, some embodiments may comprise a protruding fin, as will be discussed and shown in conjunction with FIG. 30.

FIGS. 13a,b depict an embodiment that differs from the embodiment depicted in FIGS. 12a,b in that it comprises only axial protrusions 1304 and accompanying lysing elements. In addition, some of the elements in FIGS. 13b are shown in greater detail in connection with these figures than the corresponding elements in FIGS. 12a,b. Such elements are as follows: FIG. 13b also depicts a breakaway showing elements that are present but not seen in the embodiment depicted in FIGS. 12a,b, including SC shaft conductive opening 1362.6 in SC shaft 1362, modular TD shaft conductive core 1303.7, and SC coupler conductive element 1303.9. SC coupler conductive element 1303.9 comprises a protruding electrically-conductive element. More particularly, SC coupler conductive element 1303.9 comprises a spring, such as a leaf spring, that may be used to facilitate desired contact with a portion of the SC, such as SC shaft 1362.

Each of the other elements depicted in FIGS. 13a,b may be identical to the corresponding elements shown in FIGS. 12a,b and are referenced by like numerals (numbers higher by 100). For example, FIGS. 13a,b depict an embodiment comprising an axially advanceable SC 1360. SC comprises SC shaft 1362, SC tip 1361, SC toggle 1381, SC coupler 1365 and/or SC sheath 1365.1. The modular TD 1300 depicted in this embodiment comprises a Modular TD tip 1301, Modular TD shaft 1302, Modular TD plug 1303 (in phantom) and also comprises axial protrusions 1304 but lacks non-axial protrusions and lysing segments. The standard electrosurgical ‘pencil’ depicted, comprises shaft 1302.1, ‘pencil’ switch 1303.2, ‘pencil’ handle 1303.1 and/or conduit 1311.2. SC tip 1361 may retract into SC sheath 1365.1 via SC opening 1365.4. In some embodiments, SC opening 1365.4 may be formed within SC gate 1365.3. SC shaft 1362 may be limited in excursion by limiting ridge 1366. SC toggle 1381 may comprise grip 1382. Although, the embodiment depicted in FIG. 13a shows the SC shaft 1362 pointing about axially (relative to the modular TD axis) and/or passing about over the middle of the front of the modular TD tip 1301, in other contemplated embodiments, SC shaft 1362 may point at an angle within about 30 degrees of the axis of the modular TD and/or may pass over any other points on the modular TD tip 1301 besides the middle front of the modular TD tip. In other contemplated embodiments, SC shaft 1362 may point at angles greater than 30 degrees of the axis of the modular TD.

As shown in FIG. 13b breakaway, SC 1360 is electrically coupled with modular TD 1300 by SC connecting contact 1303.9 & SC shaft conductive opening 1362.6. SC connecting contact 1303.9 may be configured to accommodate axial movement of SC shaft 1362 between the operational and storage positions. For example, some embodiments may comprise a plurality of adjacent conductive rings or other non-insulated areas such that SC tip 1361 may be positioned at a plurality of preset distances from modular TD tip. In the depicted embodiment, SC connecting contact 1303.9 is electrically coupled to the modular TD shaft 1362 conductive core 1303.7. In contemplated embodiments, SC connecting contact 1303.9 may be electrically coupled to the modular TD tip 1301 and/or modular TD plug 1302. In contemplated embodiments, the SC connecting contact 1303.9 may comprise a conductive piece emanating and/or projecting from the conductive core of modular TD shaft 1302, for example a ‘fin’; such pieces may be manufactured by stamping and/or welding or other method known in the art. An example of a protruding electrically-conductive element comprising a fin is shown and discussed in conjunction with FIG. 30 below. In some embodiments, the electrically-conductive element may be flexible, such as spring-loaded or spring-biased, to further facilitate desired electrical contact.

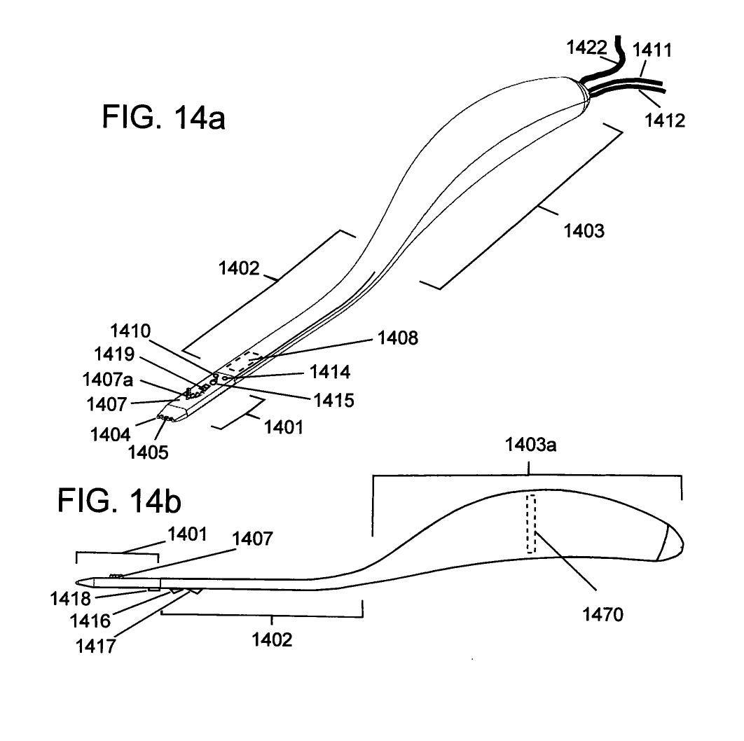

FIG. 14a is a perspective view of an embodiment of a TDM comprising a tip 1401, a shaft 1402 and a handle 1403. In the depicted embodiment, electrosurgical energy such as for example electro-coagulation and/or electro-cutting waveforms arrive in electrical conduits 1411 and/or 1412 and may travel by wiring through the handle and shaft to termini 1407a, which are part of energy window 1407. In the depicted embodiment, electrosurgically energized energy window 1407 may be present on the upper side of the device. Electro-cutting and electro-coagulation currents may be controlled outside the TDM via an electrosurgical generator, for example the Bovie Aaron 1250™ or Bovie Icon GP™. It should be noted that the term “energy window” is intended to encompass what is referred to as a planar-tissue-altering-window/zone in U.S. Pat. No. 7,494,488 and, as described later, need not be electrosurgically energized in all embodiments. In some embodiments, the “energy window” may comprise a variety of other energy emitting devices, including radiofrequency, electromagnetic, thermochromic, intense pulsed light, LASER, thermal, microwave and/or ultrasonic. It should also be understood that the term “energy window” does not necessarily imply that energy is delivered uniformly throughout the region comprising the energy window. Instead, some energy window implementations may comprise a series of termini or other regions within which energy is delivered with interspersed regions within which no energy, or less energy, is delivered. This configuration may be useful for some implementations to allow for alteration of certain tissue areas with interspersed areas within which tissue is not altered, or at least is less altered. This may have some advantages for certain applications due to the way in which such tissue heals. A second energy window 1408 may also be included in some embodiments, and may comprise yet another electrical energy emitting area. In some embodiments, the “second energy window” may comprise a variety of other energy emitting devices, including radiofrequency, thermochromic, intense pulsed light, LASER, thermal, microwave and ultrasonic. It is contemplated that in alternative embodiments, either one or both of the energy windows may be omitted.

In the depicted embodiment, the tip 1401 may measure about 1 cm in width and about 3 mm in thickness. It is contemplated that in alternative embodiments, sizes of about one-fifth to about five times these dimensions may also have possible uses. In some contemplated veterinary embodiments, tip sizes of about one-tenth to 20 times the aforementioned dimensions may also have possible uses. In some embodiments, the tip can be a separate piece that is secured to shaft by a variety of methods such as a snap mechanism, mating grooves, plastic sonic welding, etc. Alternatively, in some other embodiments, the tip can be integral or a continuation of shaft made of similar metal or materials. In some embodiments, the tip may comprise materials that are both electrically non-conductive and of low thermal conductivity; such materials might comprise, for example, ceramics, porcelain, glass-ceramics, plastics, various halogenated carbon molecules, polytetrafluoroethylene, carbon, graphite, graphite-fiberglass composites and the like. In some embodiments, the tip may be constructed of a support matrix of an insulating material (for example, ceramic, glass, alumina, zirconia).

In the depicted embodiment, external conduits 1411 and/or 1412 (which may also contain electrical control wires to aid in device operation) may connect to electrically conductive elements to bring RF electrosurgical energy from an electrosurgical generator down the shaft 1402 to electrically conductive lysing elements 1405 mounted in the recessions in between the protrusions 1404. In some embodiments, the protrusions may comprise bulbous protrusions. Partially hidden from direct view in FIGS. 14a,b, and located in the grooves defined by protrusions 1404 are electrically conductive tissue lysing elements 1405, which, when powered by an electrosurgical generator, effects lysing of tissue planes on forward motion of the device. Lysing elements may be located at the termini of conductive elements.

In the depicted embodiment, the tip 1401 may comprise, partially or completely, concentrically laminated or annealed-in wafer layers of materials that may include plastics, silicon, glass, glass/ceramics, cermets or ceramics. Lysing elements 1405 may also comprise, partially or completely, a cermet material. In an alternative embodiment, the tip may comprise insulation covered metals and/or electroconductive materials.

The tip shown in the depicted embodiment has four relative protrusions and three relative recessions & a monopolar tip conductive element. All of the axes of the relative protrusions of the tip depicted in this embodiment extend at least substantially parallel to the axis of the shaft of the TDM (as viewed from Top). In embodiments of tips of such axially oriented protrusions and/or relative recessions, surgeons may use methods of defining and or dissecting a target area by entering through an incision and then moving the TDM tip in a primarily axial direction forward and backward and reorienting the TDM after the backstroke in a spokewheel pattern the TDM to access tissues adjacent to earlier strokes.

In some embodiments, shaft 1402 may range in cross-section for example, oval, flat, rectangular, geometric or substantially flattened. In some embodiments, smoothing of the edges of the shaft may reduce friction on the skin surrounding the entrance wound. In some further embodiments, the shaft may comprise metal, plastic and/or other material. In alternative embodiments, the shaft interior may be completely occupied or hollowed containing wires, electrical conductors, fluid/gas pumping or suctioning conduits, fiber-optics, or insulation. In the depicted embodiment, shaft 1402 may be about 15 cm in length and/or handle 1403 may be about 10 cm in length. In some embodiments, the shaft may have a length of about 10-20 cm. In some embodiments, the handle may have a length of about 8-18 cm. In some embodiments, shaft plastics, such as polytetrafluoroethylene may act as insulation about wire or electrically conductive elements. In some embodiments, the shaft may alternatively be made partially or completely of concentrically laminated or annealed-in wafer layers of materials that may include plastics, silicon, glass, glass/ceramics, ceramics carbon, graphite, graphite-fiberglass composites. Depending upon the intended uses for the device, an electrically conductive element internal to shaft may be provided to conduct electrical impulses or RF signals from an external power/control unit (such as a Valleylab™ electrosurgical generator) to another energy window 1408.

In the depicted embodiment, energy windows 1407 and/or 1408 may comprise substantially planar shapes. In alternative embodiments, energy windows 1407 and/or 1408 may take on other cross-sectional shapes that may correspond with a portion of the shape of the shaft, such as arced, stair-step, or other geometric shapes/curvatures. In the depicted embodiment in FIGS. 14a,b energy window 1407 is adjacent to protrusions 1404. However, in other contemplated embodiments, an energy window may be positioned elsewhere on the shaft 1402 or tip 1401 of the wand, and still be considered adjacent to protrusions 1404. For example, in an embodiment lacking energy window 1407, but still comprising energy window 1408, energy window 1408 would still be considered adjacent to protrusion 1404. However, if an energy window was placed on handle 1403, such an energy window would not be considered adjacent to the protrusions 1404. In some embodiments, the electrosurgically energized window waveforms may be further pulsed at varying rates, by interpolating gating circuitry at some point external to the electrosurgical generator by standard mechanisms known in the art that may range from about 1 per second to about 60 per second. In some embodiments, the rate may vary from about 1 per second to about 150 per second. In some embodiments, the electrosurgically energized tip current can be further pulsed at varying rates by gating circuitry within the electrosurgical generator by standard mechanisms known in the art.

In some embodiments, the electrically conductive lysing elements portion of the tip may arise from a plane or plate of varying shapes derived from the aforementioned materials by methods known in the manufacturing art, including but not limited to additive manufacturing, cutting, stamping, pouring, molding, filing and/or sanding. In some embodiments, the electrically conductive lysing elements 1405 may comprise an insert attached to a conductive element in the shaft or continuous with a formed conductive element coursing all or part of the shaft. In some embodiments, lysing elements may be continuous and continue to extend all the way around a tip. In some embodiments lysing elements may be discrete and do not extend all the way around a tip. For example, in some embodiments each of the lysing elements may comprise a separate piece of material. In other embodiments, each of the lysing elements may comprise a portion of a single plate, wire or other piece of material. In some embodiments, one or more electrically conductive elements or wiring in conduit 1411 and/or 1412 brings RF electrosurgical energy down the shaft to electrically conductive lysing elements 1405 associated in part with the recessions. In an embodiment, the electrosurgical energy via conduit 1411 is predominately electro-cutting and/or a blend.

In some embodiments, the electrically conductive element or wiring may be bifurcated to employ hand switching if an optional finger switch is located on handle. The electrically conductive element or wiring leading from the shaft into the handle may be bundled with other electrical conduits or energy delivering cables, wiring and the like and may exit the proximal handle as insulated general wiring to various generators (including electrosurgical), central processing units, lasers and other sources as have been described herein.

In some embodiments, the plate making up lysing elements 1405 may be sharpened or scalloped or made to slightly extend outwardly from the tip recessions into which the plate will fit. Alternatively, in some embodiments, since cutting or electrical current may cause an effect at a distance without direct contact, the lysing elements may be recessed into the relative recessions or grooves defined by the protrusions 1404 or, alternatively, may be flush with protrusions 1404. In some further adjustable embodiments, locations of the electrically conductive lysing elements with respect to the protrusions may be adjusted by diminutive screws and/or ratchets. In some further adjustable embodiments, locations of the electrically conductive lysing elements with respect to the protrusions may be adjusted by MEMS or microelectronics. In the depicted embodiment, lysing element plate is about 0.5 mm in thickness. In other embodiments, the plate thickness may range from 0.1 mm to 3 mm. Plate bevel sharpness may range to varying degrees on its forward facing surface; however, sharp edges may increase the efficiency with which electricity will pass from an edge & may increase the aggressiveness of electrosurgical cutting of a target tissue.

In some implementations, the electrosurgical current for the electrically conductive lysing elements may comprise monopolar “cutting,” “blend” &/or “coag” selections. In some embodiments, the electrosurgical current for the electrically conductive lysing elements may be delivered to the tip lysing conductor in a continuous fashion or, alternatively, a pulsed fashion. Surgeons may control the presence of current by a foot pedal control of the electrosurgical generator and/or by button control on the shaft and/or handle. The amount of electrosurgical current and waveform may be modified by standard interfaces or dials on the electrosurgical generator. For monopolar embodiments, the electrosurgical generator is connected to a grounding or dispersive plate which may be placed elsewhere in contact with the patient's body, such as the thigh. Such circuitry may be controlled and gated/wired from the cutting current delivery system of the electrosurgical generator. Acceptable electrosurgical generators may include Valley Lab Force 1 B™ with maximum P-P voltage of 2400 on “cut” with a rated load of 300 Ohms and a maximum power of 200 Watts, 35 maximum P-P voltage of 5000 on “coagulate” with a rated load of 300 Ohms, and a maximum power of 75 Watts ValleyLab Force 4 has a maximum P-P voltage of 2500 on “cut” with a rated load of 300 Ohms and a maximum power of 300 Watts, 750 kHz sinusoidal waveform output, maximum P-P voltage of 9000 on “coagulate” with a rated load of 300 Ohms and a maximum power of 120 Watts using a 750 kHz damped sinusoidal with a repetition frequency of 31 kHz.

In the depicted embodiment, tip 1401 may comprise materials that are both electrically non-conductive and of low thermal conductivity such as porcelain, epoxies, ceramics, glass-ceramics, plastics, or varieties of polytetrafluoroethylene. In an alternative embodiment, the tip may comprise multilayer wafer substrates comprised of bonded conductive strips and ceramics. Suitable conductive materials may comprise those already described for tip manufacture. In alternative embodiments, the tip may be made from metals or electroconductive materials that are completely or partially insulated. In the depicted embodiment, positioned in the relative recessions of the tip are planar, crescent shaped (concave), stainless steel, electrically conductive tissue lysing elements 1405 (usually hidden from view at most angles). Note the relative protrusions and relative recessions may not be completely visible from certain viewing angles. In contemplated embodiments, lysing element shapes may be convex, straight and/or virtually any geometric shape. In contemplated embodiments, electrically conductive lysing elements may be in the shape of a plate, plane and/or wire. In contemplated embodiments, electrically conductive lysing elements may comprise conductive materials may comprise but are not limited to steel, nickel, alloys, palladium, gold, tungsten, titanium, silver, copper, and platinum and the like. In contemplated embodiments, electrically conductive lysing elements may comprise a conductive material and/or metal that does not melt under operating conditions or give off toxic residua. In alternative embodiments the geometry of the tip area may comprise protrusions that are not oriented along the axis of the shaft (for example, as seen in FIGS. 3, 4, 5 herein, 351b, 451b, 551.1). Certain metals may become oxidized after repeated use, thus impeding electrical flow and function. Some embodiments may comprise a low cost, disposable, and one-time-use device. However, in some embodiments intended for multiple uses, the tip's electrically conductive tissue lysing elements be protected or coated with materials that include, but are not limited to, Silverglide™ non-stick surgical coating, platinum, palladium, silver, gold and rhodium. Varying the amount of protective coating allows for embodiments of varying potential for obsolescence capable of either prolonging or shortening instrument life. Some embodiments may be configured to be modular and/or comprise disposable tips such that a surgeon can place an appropriate tip for a particular surgery on the shaft. Alternatively or additionally one or more of the tips may be disposable such that a surgeon may dispose of the tip after performing surgery and install a new tip for subsequent surgeries or a continuation of the current surgery with a new tip.

In alternative embodiments, the electrically conductive lysing elements may be bifurcated or divided into even numbers at the relative recessions, insulated and energized by wiring to an even number of electrical conduits in a bipolar fashion and connected to the bipolar outlets of the aforementioned electrosurgical generators. Rings partly or completely encircling the shaft of the hand unit can be linked to a partner bipolar electrode at the tip or on the energy window. Such bipolar versions may decrease the energy necessary to electrically modify certain tissues, especially thicker tissues. In alternative embodiments, the lysing elements may be divided into odd numbers yet still allow for bipolar flow between two or more elements as those of ordinary skill in the art would appreciate.

In some embodiments, one or more suction/vacuum ports 1417 may be provided on or about the tip or distal shaft. The port(s) may be fluidly coupled with a vacuum; the vacuum may comprise a pump or a negative pressure chamber or a syringe at the end of a fluid conduit. Other embodiments may comprise one or more suction/vacuum ports on any other suitable location on the TDM, including but not limited to on the protrusions or otherwise on the tip, and on the shaft. In some embodiments, a fluid delivery port 1416 may be provided. In some embodiments the fluid delivery port may be coupled with a pump or high pressure fluid. In some embodiments the port may be perpetually open such that fluid may be delivered therethrough upon actuation of a pump or fluid pressure system. In other embodiments the port may be closed and selectively opened to deliver fluid therethrough. Other embodiments may comprise one or more fluid ports on any other suitable location on the TDM, including but not limited to on the protrusions or otherwise on the tip, and on the shaft. Fluid ports that may be useful may comprise channels within the TDM, polymer lines, hoses, etc. Fluids that may emanate from the outlet may comprise ionic fluids such as saline, medicines (including but not limited to antibiotics, anesthetics, antineoplastic agents, bacteriostatic agents, etc.), non-ionic fluids, and or gasses (including but not limited to nitrogen, argon, air, etc.). In some embodiments fluids may be under higher pressures or sprayed. It should be understood that although these elements (1416 & 1417) are not depicted in every one of the other figures, any of the embodiments described herein may include one or more such elements.

In some embodiments, a vibration means 1470 may be positioned in the handle. Other embodiments may comprise one or more vibration means on any other suitable location on the TDM, including but not limited to on the protrusions or otherwise on the tip, and on the shaft. Examples of suitable vibration means may include piezoelectric materials, ultrasonic motors with stators, piezoelectric actuators, vibration motor such as an off-center weight mounted on a gear, etc. Some vibration means may be configured to emit ultrasound in the 20-40 kHz range. Yet other vibration means may include electromagnet drivers with a frequency of operation in the range of 150-400 Hz. In some embodiments, one or more vibration means may be used to provide additional forces which may facilitate passage of the TDM. In some embodiments, one or more vibration means may be used to reduce debris on the electrosurgical or other components of the TDM. In a further embodiment, a vibration means may be directly or indirectly connected to one or more of the lysing elements. Some vibration means may help to decrease and/or remove debris. In some embodiments use of a vibration means may, also or alternatively, be used to assist in migrating the TDM through tissue during the procedure. In some such embodiments, it is thought that use of a vibration means having a lower frequency may be particularly useful for assisting in such migration. In addition, positioning the vibration means closer to a handle of the TDM may facilitate such migration as well. By contrast, positioning the vibration means on or near the tip, and/or using a higher frequency vibrations means may be particularly useful for preventing buildup of debris on the tip.

In some embodiments, one or more electromagnetic delivery elements 1415 may be positioned on tip or shaft. Other embodiments may comprise one or more electromagnetic delivery elements on any other suitable location on the TDM, including but not limited to on the protrusions or otherwise on the tip, and on the shaft. Electromagnetic delivery elements that may be useful include: LEDs, LASERs, fiberoptics, filaments, photoelectric materials, infrared emitters, etc.

The sensors 1410 and 1414 may comprise any of the sensors described in the specification herein. Other embodiments may comprise one or more sensors on any other suitable location on the TDM, including but not limited to on the protrusions or otherwise on the tip, and on the shaft. Sensors that may be useful include thermal sensors, photoelectric or photo optic sensors, cameras, etc. In some embodiments, one or more sensors may be used to monitor the local post passage electrical impedance or thermal conditions that may exist near the distal tip of the shaft or on the tip. Some embodiments may also comprise one or more sensors incorporating MEMS (Micro Electro-Mechanical Systems) technology, such as MEMS gyroscopes, accelerometers, galvanometers, piezoelectrics, mechanical scanning elements, diffractive elements, acousto-optic elements, capacitive sensor arrays and the like. Such sensors may be positioned at any number of locations on the TDM, including within the handle in some embodiments. In some embodiments, sensor 1414 may comprise fiberoptic elements. In an embodiment, the sensor can be configured to sense a temperature of tissue adjacent to the apparatus. The temperature sensor may alternatively be configured or sense a temperature of one or more fluids adjacent to the apparatus such as for example tissue fluids and/or fluids introduced by the surgeon.

Temperature and impedance values may be tracked on a display screen or directly linked to a microprocessor capable of signaling control electronics to alter the energy delivered to the tip when preset values are approached or exceeded. Typical instrumentation paths are widely known, such as thermal sensing thermostats, and may feed to analog amplifiers which, in turn, feed analog digital converters leading to a microprocessor. In some embodiments, internal or external ultrasound measurements may also provide information which may be incorporated into a feedback circuit. In an embodiment, an optional mid and low frequency ultrasound transducer may also be activated to transmit energy to the tip and provide additional heating and may additionally improve lysing. In some embodiments, a flashing visible light source, for example, an LED, can be mounted on the tip may show through the tissues and/or organs to identify the location of the device.

Temperature and impedance values may be tracked on a display screen or directly linked to a microprocessor capable of signaling control electronics to alter the energy delivered to the tip when preset values are approached or exceeded. Typical instrumentation paths are widely known, such as thermal sensing thermistors, and may feed to analog amplifiers which, in turn, feed analog digital converters leading to a microprocessor. In some embodiments, internal or external ultrasound measurements may also provide information which may be incorporated into a feedback circuit. In an embodiment, an optional mid and low frequency ultrasound transducer may also be activated to transmit energy to the tip and provide additional heating and may additionally improve lysing. In some embodiments, a flashing visible light source, for example, an LED, can be mounted on the tip may show through the tissues and/or organs to identify the location of the device.

In some embodiments, one or more electromagnetic delivery elements 1415 may be positioned on tip or shaft. Other embodiments may comprise one or more electromagnetic delivery elements on any other suitable location on the TDM, including but not limited to on the protrusions or otherwise on the tip, and on the shaft. Electromagnetic delivery elements that may be useful include: LEDs, LASERs, fiberoptics, filaments, photoelectric materials, infrared emitters, etc.

TDM tip 1401 may further comprise Internal Tissue Optical Motion Sensor (ITOMSensor) 1419. ITOMSensor 1419 may comprise one or more lenses and/or windows for emitting EMR and may further comprise one or lenses and/or windows for capturing or receiving reflected EMR. In some contemplated embodiments, TDM shaft 1402 may instead comprise ITOMSensor 1419.

In order to improve the ability of the TDM to track movement with respect to an internal tissue or region of tissue, such as the fatty undersurface of the skin, some embodiments may comprise an ITOMSensor comprising an electromagnetic emission source and a photodetector device. In, alternative embodiments, the internal tissue optical motion sensor may comprise a non-coherent light source. In, the depicted embodiment, the ITOMSensor comprises a coherent light source. Some such embodiments may comprise a motion detection algorithm configured to extract peaks & nulls for detected speckled light intensity patterns. One example of such an algorithm is disclosed in U.S. Pat. No. 7,876,307 titled ‘Motion Detection Mechanism for Laser Illuminated Optical Mouse Sensor’ which is hereby incorporated by reference in its entirety Other examples of an optical sensor that may be used as an ITOMSensor are disclosed in US Patent Application publication number 2013/0016041 titled ‘High Resolution Mouse’ which is hereby incorporated by reference in its entirety. Other examples an optical sensor that may be used as an ITOMSensor are disclosed in: U.S. Pat. No. 7,791,590B1 titled: ‘Optical Mouse With Uniform Level Detection & U.S. Pat. No. 5,578,813 titled: ‘Freehand Image Scanning Device Which Compensates For Non-Linear Movement’ & U.S. Pat. No. 5,644,139 titled: ‘Navigation For Detecting Movement Of Navigation Sensors Relative To An Object’ & U.S. Pat. No. 5,786,804 titled: ‘Method and System For Tracking Attitude,’ which are hereby incorporated by reference in their entirety. One or more steps, features, elements, or components disclosed in any of the above referenced documents which have been incorporated herein by reference may be combined with any other step disclosed in any of the other steps, features, elements, or components disclosed in any of the other documents disclosed herein incorporated by reference herein as would be apparent to one of ordinary skill in the art.

An ITOMSensor may comprise a source of non-coherent light which may be part of the motion sensor or located elsewhere on the surgical tool for illuminating a tissue at a low angle of incidence, a two dimensional array of photodetectors, each of the photo detectors producing an output in response to light reflected from surface irregularities in the tissue, and circuitry (which may be in the surgical tool or located remotely) to track movement of the housing relative to the work surface by comparing at least some of the photo detector outputs sensed at a first time with at least some of the photo detector outputs sensed at a second time if a particular condition in the photo detector outputs is identified. Motion produces successive frames of translated patterns of pixel information, which may be compared to determine the direction and distance moved.

One method for motion detection that may be used in connection with one or more embodiments disclosed herein may be based on based on the “Peak/Null Motion Detection” algorithm described in the International Patent Application WO_{—}03/049018 which reference is incorporated by reference herein in its entirety.

According to the “Peak/Null Motion Detection” algorithm a distinction is made between edges according to their “direction” which may be referred to as edge direction data. In some embodiments based upon the Peak/Null Motion Detection Algorithm, the ITOMSensor may comprise a coherent light source and a photodetector array. In connection with such embodiments the ITOMS may operate by illuminating under a determined gradient using a coherent light source a portion of tissue at a determined flash rate; detecting by means of the photodetector array a reflected speckled light intensity pattern from the illuminated portion of the internal tissue surface for a first flash; detecting a second reflected speckled light intensity pattern of the illuminated portion of the internal tissue surface for a second flash; extracting motion features of two different types from the detected first and second speckled light intensity patterns; determining a measurement of the relative motion between the surgical tool and the illuminated portion of the internal tissue surface based on a comparison of motion features extracted. In some embodiments, before the step of determining the measurement, preliminary step(s) (may be executed) consisting of modifying/adjusting the gradient under which the surface is illuminated In some embodiments, the method may comprise keeping only pairs of neighboring Peaks and Nulls and ignoring individually occurring Peaks and Nulls.

One or more steps, features, elements, or components disclosed in any of the above referenced documents which have been incorporated herein by reference may be combined with any other step disclosed in any of the other steps, features, elements, or components disclosed in any of the other documents disclosed herein incorporated by reference herein as would be apparent to one of ordinary skill in the art.

In some embodiments Internal Tissue Optical Motion Sensor 1419 may comprise for example one or more elements described in the aforementioned incorporation by reference. In alternative embodiments ITOMSensor 1419 may be located elsewhere on modular TDM 1400 such as for example on shaft 1402. In some embodiments, ITOMSensor 1419 may be coupled with lysing elements and/or energy window to facilitate controlled delivery of energy to tissues. For example, some embodiments may be configured to deliver a preset amount of energy such that when the device is moved faster a larger amount of energy is delivered per unit time, and when the device is moved more slowly the amount of energy is automatically decreased so as to provide a more uniform or at least semi-uniform (at least within a predetermined range) delivery of energy per unit area. In some embodiments, motion 1419 sensor may further or alternatively be coupled with sensor 1410 and/or 1414 for example, in embodiments comprise a temperature sensor; temperature data may be used along with data from ITOMSensor 1419 in order to adjust energy output to energy window 1407 and/or lysing elements.

In the depicted embodiment, 1418 represents an antenna configured to deliver a signal to a receiver unit. In some embodiments, antenna 1418 may comprise radiofrequency identification (RFID) TAG. In some embodiments the RFID tag may comprise an RFID transponder. In other embodiments the RFID tag may comprise a passive tag. It should be understood that antenna 1418 is not depicted in every one of the other figures; any of the embodiments described herein may comprise one or more such elements. Other embodiments may comprise one or more antenna on any other suitable location on the TDM, including but not limited to on the protrusions or otherwise on the tip, and on the shaft. In embodiments in which antenna 1418 comprises an RFID transponder, the RFID transponder may comprise a microchip, such as a microchip having a rewritable memory. In some embodiments, the tag may measure less than a few millimeters. In some embodiments a reader may generate an alternating electromagnetic field which activates the RFID transponder and data may be sent via frequency modulation. In an embodiment, the position of the RFID tag or other antenna may be determined by an alternating electromagnetic field in the ultra-high frequency range. The position may be related to a 3 dimensional mapping of the subject. In an embodiment the reader may generate an alternating electromagnetic field. In some such embodiments, the alternating electromagnetic field may be in the shortwave (13.56 MHz) or UHF (865-869 MHz) frequency. Examples of potentially useful systems and methods for mapping/tracking a surgical instrument in relation to a patient's body may be found in U.S. Patent Application Publication No. 2007/0225550 titled “System and Method for 3-D Tracking of Surgical Instrument in Relation to Patient Body, which is hereby incorporated by reference in its entirety.

In some embodiments, a transmission unit may be provided that may generate a high-frequency electromagnetic field configured to be received by an antenna of the RFID tag or another antenna. The antenna may be configured to create an inductive current from the electromagnetic field. This current may activate a circuit of the tag, which may result in transmission of electromagnetic radiation from the tag. In some embodiments, this may be accomplished by modulation of the field created by the transmission unit. The frequency of the electromagnetic radiation emitted by the tag may be distinct from the radiation emitted from the transmission unit. In this manner, it may be possible to identify and distinguish the two signals. In some embodiments, the frequency of the signal from the tag may lie within a range of the frequency of the radiation emitted from the transmission unit. Additional details regarding RFID technology that may be useful in connection with one or more embodiments discussed herein may be found in, for example, U.S. Patent Application Publication No. 2009/0281419 titled “System for Determining the Position of a Medical Instrument,” the entire contents of which are incorporated herein by specific reference.

In other embodiments, antenna 1418 may comprise a Bluetooth antenna. In such embodiments, multiple corresponding Bluetooth receivers at known locations may be configured to sense signal strengths from the Bluetooth antenna 1418 and triangulate such data in order to localize the signal from the Bluetooth antenna 1418 and thereby locate the TDM within a patient's body. Other embodiments may be configured to use angle-based, electronic localization techniques and equipment in order to locate the antenna 1418. Some such embodiments may comprise use of directional antennas, which may be useful to increase the accuracy of the localization. Still other embodiments may comprise use of other types of hardware and/or signals that may be useful for localization, such as WIFI and cellular signals, for example.

One or more receiver units may be set up to receive the signal from the tag. By evaluating, for example, the strength of the signal at various receiver units, the distances from the various receiver units may be determined. By so determining such distances, a precise location of the TDM relative to a patient and/or a particular organ or other surgical site on the patient may be determined. In some embodiments, a display screen with appropriate software may be coupled with the RFID or other localization technology to allow a surgeon to visualize at least an approximate location of the tag/antenna, and therefore TDM, relative to the patient's body.

Some embodiments may be further configured such that data from the antenna(s) may be used in connection with sensor data from the TDM. For example, some embodiments of TDMs comprising one or more sensors may be further configured with one or more RFID tags. As such, data from the one or more sensors may be paired or otherwise used in connection with data from the one or more RFID tags or other antennas. For example, some embodiments may be configured to provide information to a surgeon regarding one or more locations on the body from which one or more sensor readings were obtained. To further illustrate using another example, information regarding tissue temperature may be combined with a location from which such tissue temperature(s) were taken. In this manner, a surgeon may be provided with specific information regarding which locations within a patient's body have already been treated in an effective manner and thus which locations need not receive further treatment using the TDM.

In some such embodiments, a visual display may be provided comprising an image of the patient's body and/or one or more selected regions of a patient's body. Such a system may be configured so as to provide a visual indication for one or more regions within the image corresponding to regions of the patient's tissue that have been sufficiently treated. For example, a display of a patient's liver may change colors at locations on the display that correspond with regions of the liver that have experienced a sufficient degree of fibrosis or other treatment. Such regions may, in some embodiments, be configured such that pixels corresponding to particular regions only light up after the corresponding tissue in that region reaches a particular threshold temperature.

Such sensors 1410 and/or 1414 may be coupled with an antenna, which may send and/or receive one or more signals to/from a processing unit. Alternatively, or additionally, data from such sensors resulting from tissue and/or fluid analysis using such sensors may be stored locally and transmitted later. As yet another alternative, such a signal may be transmitted following surgery. In such implementations, the signals need not necessarily be transmitted wirelessly. In fact, some embodiments may be configured to store data locally, after which a data module, such as a memory stick, may be removed from the TDM and uploaded to a separate computer for analysis.

In some embodiments tip 1401 may be attached to a robotic arm. In some embodiments, tip 1401 and portion of shaft 1402 may be attached to a robotic arm. In some embodiments, tip 1401 and/or a portion of shaft 1402 and/or a portion shaft and/or portion of handle 1403 may be attached to a robotic arm. In some embodiments, the robotic arm may comprise one or more motors such as a screw-drive motor, gear motor, hydraulic motors, etc. In some embodiments the robotic arm system may comprise worm gearheads, video cameras, motor control circuits, monitors, remote control devices, illumination sources, tactile interface, etc.

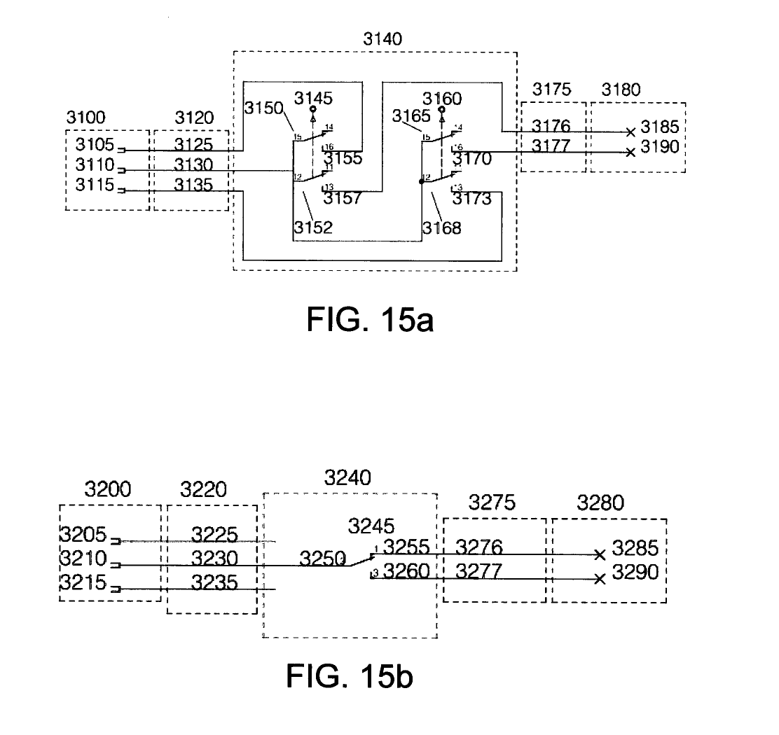

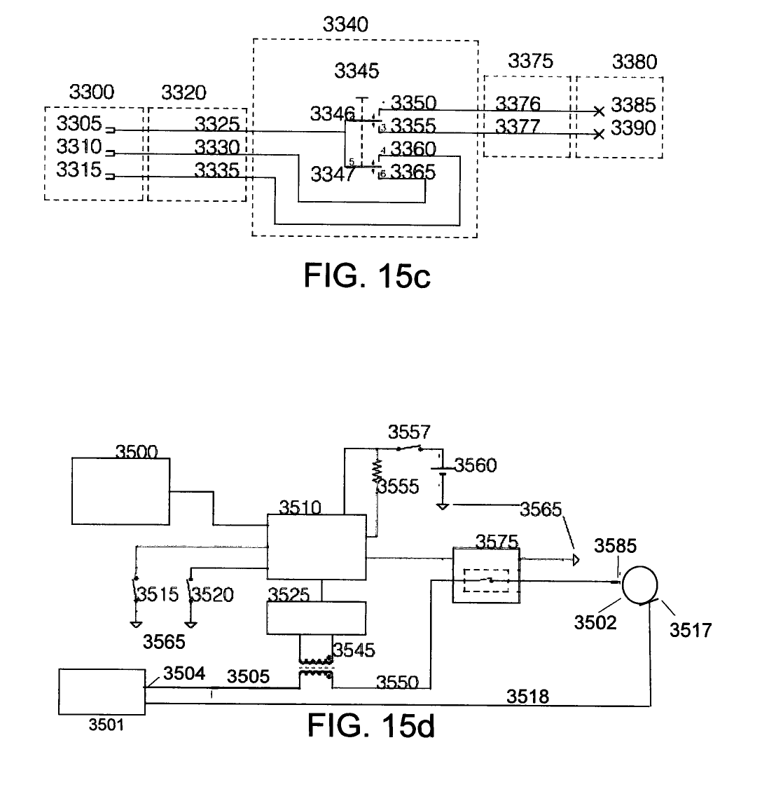

FIG. 15a, depicts an embodiment wherein the electrical wiring for the TDM comprises a connector 3100, a three-conductor cable 3120, a first DPST (Double-pole, Single-throw) switch 3145 that may be used to deliver a coagulation waveform (“COAG DPST switch”), a second DPST switch 3160 that may be used to deliver a cutting waveform (“CUT DPST switch”), a shaft 3175 with two conductors 3176 and 3177, and a tip 3180 comprised of a first electrode 3185 that may be used for tissue modification and/or coagulation (“COAG electrode”), and a second electrode 3190 that may be used for cutting tissue and/or tissue coagulation (“CUT electrode”). It is well known in the art that to complete a monopolar circuit either electrode may make contact with the patient who is in contact with a conductive return pad connected to the energy source. The three-conductor cable 3120 comprises one RF conductor 3130, one CUT switch conductor 3135, and one COAG switch conductor 3125. The RF conductor 3130 extends from the RF pin 3110, positioned in the connector 3100, to the RF pins 3150 & 3152 of the COAG DPST switch 3145 and the RF pins 3165 & 3168 of the CUT DPST switch 3160 which may be positioned in the handle 3140. The CUT switch conductor 3135 extends from the CUT switch pin 3115, positioned in the connector 3100, to the CUT switch pin 3173 of the CUT DPST switch 3160 which may be positioned in the handle 3140. The COAG switch conductor 3125 extends from the COAG switch pin 3105, positioned in the connector 3100, to the COAG switch pin 3155 of the COAG DPST switch 3145 which may be positioned in the handle 3140. The COAG electrode conductor 3176 positioned in the shaft 3175 extends from the COAG electrode 3185 positioned in the tip 3180, to the COAG electrode pin 3157 of the COAG DPST switch 3145. The CUT electrode conductor 3177, positioned in the shaft 3175, extends from the CUT electrode 3190 positioned in the tip 3180, to the CUT electrode pin 3170 of the CUT DPST switch 3160. When the COAG DPST switch 3145 is engaged, the RF pins 3150 & 3152 are electrically short circuited to the COAG switch pin 3155 and the COAG electrode pin 3157 simultaneously. When the CUT DPST switch 3160 is engaged, the RF pins 3165 & 3168 are electrically short circuited to the CUT switch pin 3173 and the CUT electrode pin 3170 simultaneously. When the COAG DPST switch 3145 is not engaged, there is electrical isolation between the short-circuited RF pins 3150 & 3152, the COAG switch pin 3155, and the COAG electrode pin 3157. When the CUT DPST switch 3160 is not engaged, there is electrical isolation between the short-circuited RF pins 3165 & 3168, the CUT switch pin 3173, and the CUT electrode pin 3170. When the connector 3100 is plugged into an electrosurgical generator (for example, a Valley Lab Force FX) and the COAG DPST switch 3145 is engaged, RF energy is passed through the RF conductor 3130 to the COAG electrode 3185. Alternatively, if the CUT DPST switch 3160 is engaged while the connector 3100 is plugged into an electrosurgical generator, RF energy is passed through the RF conductor 3130 to the CUT electrode 3190. In this manner, the RF energy delivery can be turned on and off from the handle 3140. In other embodiments, the conductor cable 3120 comprises two conductors to accommodate electrosurgical generators utilizing two-pronged connectors or may comprise more conductors to accommodate other attributes of the device. In other embodiments, the DPST switches 3145 & 3160 may be positioned on the conductor cabling 3120 between the connector 3100 and the handle 3140; in such embodiments the electrode conductors 3176 & 3177 leading to the electrodes 3185 & 3190 may extend through or around the shaft 3175 & handle 3140 & may comprise at least part of conductor cabling 3120. In other embodiments, the shaft 3175 may contain more than 2 conductors. In alternative embodiments, each conductor within the conductor cable 3120 may be contained in its own cabling. In an alternative embodiment, each switch may have more than 2 positions.

In another embodiment depicted in FIG. 15b, the electrical wiring for the TDM comprises a connector 3200, a conductor cable 3220 that may comprise one or more conductors, a SPDT (Single-Pole, Double-Throw) switch 3245, a shaft 3275 with two conductors 3276 and 3277, a tip 3280 comprised of a first electrode 3285 that may be used for tissue modification and/or coagulation (“COAG electrode”), and a second electrode 3290 that may be used for cutting tissue and/or tissue coagulation (“CUT electrode”). It is well known in the art that to complete a monopolar circuit either electrode may make contact with the patient who is in contact with a conductive return pad connected to the energy source. The conductor cable 3220 comprises at least one RF conductor 3230 and may comprise one CUT switch conductor 3235, and/or one COAG switch conductor 3225. The RF conductor 3230 may extend from the RF pin 3210, positioned in the connector 3200, to the RF pin 3250 of the SPDT switch 3245 positioned in the handle 3240. The CUT switch conductor 3235 may extend from the CUT switch pin 3215, positioned in the connector 3200, to the handle 3240. The COAG switch conductor 3225 may extend from the COAG switch pin 3205, positioned in the connector 3200, to the handle 3240. Alternatively, the COAG switch conductor 3225, the CUT switch conductor 3235, the COAG switch pin 3205, and the CUT switch pin 3215 may be omitted from this embodiment. The COAG electrode conductor 3276 positioned in the shaft 3275 may extend from the COAG electrode 3285 positioned in the tip 3280, to the COAG electrode pin 3255 of the SPDT switch 3245. The CUT electrode conductor 3277, positioned in the shaft 3275, may extend from the CUT electrode 3290 positioned in the tip 3280 to the CUT electrode pin 3260 of the SPDT switch 3245. The SPDT switch 3245 may be manipulated between two positions. In one switch position (the “COAG position”) the RF pin 3250 may be electrically short circuited to the COAG electrode pin 3255. In the other switch position (the “CUT position”), the RF pin 3250 may be electrically short circuited to the CUT electrode pin 3260. This embodiment may be used in conjunction with another switch such as a foot switch (for example, the Valley Lab Monopolar Footswitch, E6008) which may be coupled with an electrosurgical generator (ESU) to activate/deactivate the RF energy delivered to the TDM. In some embodiments, if the foot switch is a two-pedal variant, the type of waveform that is delivered to the RF conductor 3230 when the connector 3200 is plugged into an ESU may be determined by which pedal on the footswitch is engaged. In this manner, this embodiment enables both the CUT and COAG waveform from the ESU to be delivered to the CUT electrode 3290 and/or the COAG electrode 3285 depending on the SPDT switch 3245 position. In other embodiments, the conductor cable 3220 comprises 4 or more conductors to accommodate other attributes of the device. In other embodiments, the SPDT switch 3245 may be positioned on the conductor cabling 3220 between connector 3200 and the handle 3240; in such embodiments the electrode conductors 3276 and 3277 leading to the electrodes 3285 and 3290 may extend through or around the shaft 3275 and the handle 3240 and may comprise at least part of the conductor cabling 3220. In other embodiments, the shaft 3275 may contain more than 2 conductors. In alternative embodiments, each conductor within the conductor cable 3220 may be contained in its own cabling. In an alternative embodiment, the switch may have more than 2 positions.

In another embodiment depicted in FIG. 15c, the electrical wiring for the TDM comprises a connector 3300, a three-conductor cable 3320, a DPDT (Double-pole, Double-throw) switch 3345, a shaft 3375 with two conductors 3376 and 3377, a tip 3380 comprised of a first electrode 3385 that may be used for tissue modification and/or coagulation (“COAG electrode”), and a second electrode 3390 that may be used for cutting tissue and/or tissue coagulation (“CUT electrode”). It is well known in the art that to complete a monopolar circuit either electrode may make contact with the patient who is in contact with a conductive return pad connected to the energy source. The three-conductor cable 3320 comprises one RF conductor 3325, one CUT switch conductor 3330, and one COAG switch conductor 3335. The RF conductor 3325 extends from the RF pin 3305, positioned in the connector 3300, to the RF pins 3346 & 3347 of the DPDT switch 3345 which may be positioned in the handle 3340. The CUT switch conductor 3330 extends from the CUT switch pin 3310, positioned in the connector 3300, to the CUT switch pin 3365 of the DPDT switch 3345. The COAG switch conductor 3335 extends from the COAG switch pin 3315, positioned in the connector 3300, to the COAG switch pin 3360 of the DPDT switch 3345. The COAG electrode conductor 3376, positioned in the shaft, 3375 extends from the COAG electrode 3385 which may be positioned in the tip 3380, to the COAG electrode pin 3350 of the DPDT switch 3345. The CUT electrode conductor 3377, positioned in the shaft 3375, extends from the CUT electrode 3390 which may be positioned in the tip 3380, to the CUT electrode pin 3355 of the DPDT switch 3345. The DPDT switch 3345 may be manipulated between three positions: position one, position two, or position three. In position one, the RF pins 3346 & 3347 may be electrically short circuited to the COAG switch pin 3360 and the COAG electrode pin 3350 simultaneously. In position two, the RF pins 3346 & 3347 may be electrically short circuited to the CUT switch pin 3365 and the CUT electrode pin 3355 simultaneously. In position three, there is electrical isolation between/among the short-circuited RF pins 3346 & 3347, the COAG switch pin 3360, the COAG electrode pin 3350, the CUT switch pin 3365, and the CUT electrode pin 3355. When the connector 3300 is plugged into an electrosurgical generator (for example, a Valley Lab Force FX) and the DPDT switch 3345 is in position one, RF energy of a ‘coag’ waveform passes through the RF conductor 3325 to the COAG electrode 3385. Alternatively, if the DPDT switch 3345 is in position two while the connector 3300 is plugged into an electrosurgical generator, RF energy of a ‘cutting’ or ‘blended’ waveform passes through the RF conductor 3325 to the CUT electrode 3390. If the DPDT switch 3345 is in position three, no RF energy is delivered from the electrosurgical generator to the CUT or COAG electrodes 3385 & 3390. In this manner, RF energy delivery can be turned on and off from the handle 3340 with one switch. In other embodiments, the conductor cable 3320 comprises two conductors to accommodate electrosurgical generators utilizing two-pronged connectors or comprises more conductors to accommodate other attributes of the device. In other embodiments, the DPDT switch 3345 may be positioned on the conductor cabling 3320 between the connector 3300 and the handle 3340; in such embodiments the electrode conductors 3376 and 3377 leading to the electrodes 3385 and 3390 may extend through or around the shaft 3375 and handle 3340 and may comprise at least part of conductor cabling 3320. In other embodiments, the shaft 3375 may contain more than 2 conductors. In alternative embodiments, each conductor within the conductor cable 3220 may be contained in its own cabling. In an alternative embodiment, the switch may have more than 2 positions.

The embodiment depicted in FIG. 15d may be combined with the embodiments shown in FIGS. 15a, 15b, 15c to form the electrical wiring for the TDM. This embodiment may comprise a display 3500, a PEM (Programmable Electronic Device) 3510, an increment switch 3515, a decrement switch 3520, a filter circuit 3525, a current sense transformer 3545, a pull-up resistor 3555, a power switch 3557, a battery 3560, and a RF relay 3575. The circuit depicted in FIG. 15d may be positioned in the source of RF energy 3501, in the device handle, or as a separate enclosure between the source of energy 3501 and the connector of the RF conductor cable 3505, or in line with the RF conductor (here 3505) shown in FIGS. 15a, 15b, and 15c. This embodiment may utilize a PEM 3510 and relay 3575 to deterministically switch the RF energy on and off in order to modulate the RF energy delivered by the electrosurgical generator to electrode 3585. The frequency modulation or duty cycle may be user selectable by engaging the increment switch 3515 or the decrement switch 3520. The user selectable pulse width modulation may range from continuously on (no modulation) to a predetermined time based pulse width (e.g., 200 Hz) with a duty cycle that may range from 1% to 100%, and a respective value may be visible on the display 3500 to indicate the selected setting. Once the power switch 3557 is engaged, the microcontroller switches the RF relay 3575 on and off with a modulation selected by the user. The battery 3560 may be included to provide power to the microcontroller 3510, the filter circuit 3525, the display 3500, and the relay 3575. The current sense transformer 3545 (which could also be housed in PEM 3510) and the filter circuit 3525 may be included to facilitate feedback to the microcontroller 3510 about the electrical current delivered through the RF conductor 3550. As electrical current passes through the RF conductor, a current is induced on the current sense transformer 3545, which may then be scaled and filtered by the filter circuit 3525 in order to be used by the PEM 3510 to determine the status of the electrical current flowing through the RF conductor 3550. It is well known in the art that to complete a monopolar circuit electrode 3585 may make contact with the patient 3502 who is in contact with a conductive return pad 3517 connected to the energy source 3501 via conductor 3518. It is further well known in the art the use of signal grounds 3565 to complete various circuits.

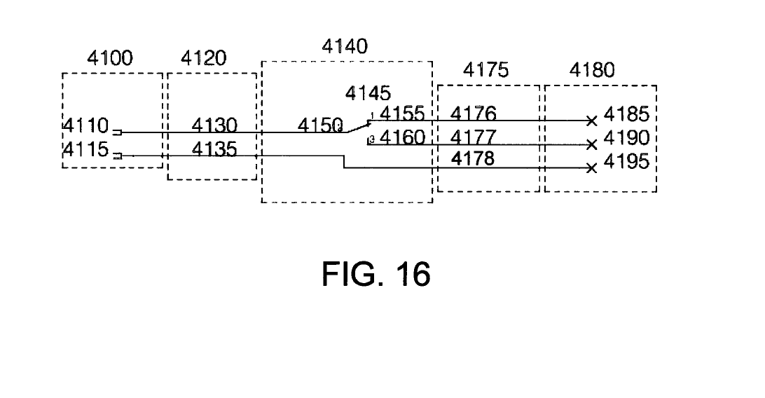

In another embodiment depicted in FIG. 16, the electrical wiring for the TDM comprises a connector 4100, a two-conductor cable 4120, a SPDT (Single-Pole, Double-Throw) switch 4145, a shaft 4175 with three conductors 4176, 4177, and 4178, a tip 4180 comprised of a first electrode 4185 that may be used for tissue modification and/or coagulation (“COAG electrode”), a second electrode 4190 that may be used for cutting tissue and/or tissue coagulation (“CUT electrode”), and a third electrode 4195 (“return electrode”). The two-conductor cable 4120 is comprised of RF conductor one 4130 and RF conductor two 4135. RF conductor one 4130 extends from RF pin one 4110, positioned in the connector 4100, to the Switch RF pin 4150 of the SPDT switch 4145 which may be positioned in the handle 4140. The COAG electrode conductor 4176 positioned in the shaft 4175 extends from the COAG electrode 4185 may be positioned in the tip 4180, to the COAG electrode pin 4155 of the SPDT switch 4145. The CUT electrode conductor 4177, positioned in the shaft 4175, extends from the CUT electrode 4190 positioned in the tip 4180, to the CUT electrode pin 4160 of the SPDT switch 4145. The RF conductor two 4135, starting in the connector 4100, extends from RF pin two 4115 that may be positioned in the connector 4100 or within another connector plugged into the electrosurgical generator, to the return electrode 4195 positioned in the tip 4180 or on the shaft 4175. The SPDT switch 4145 can be manipulated between two positions. In one switch position the switch RF pin 4150 may be electrically short circuited to the COAG electrode pin 4155. In the other switch position, the RF pin 4150 may be electrically short circuited to the CUT electrode pin 4160. This embodiment requires the use of a foot switch (such as the Valley Lab Bipolar Footswitch, E6008) in conjunction with an electrosurgical generator (ESU) to activate/deactivate the RF energy delivered to the TDM. This embodiment allows RF energy from the ESU to be delivered to the CUT electrode 4190 or the COAG electrode 4185 depending on the selected SPDT switch 4145 position. If the footswitch is engaged, electric current is able to flow between the selected electrode (CUT electrode 4190 or COAG electrode 4185) and the return electrode 4195, provided the return electrode 4195 and one of the other electrodes both make physical contact with a contiguous, electrically conductive material (As well, plasma generation may extend the reach of the CUT or COAG electrode to effectively enable contact with the contiguous, electrically conductive material). In this embodiment, the TDM may be utilized as a bipolar device with a selectable electrode configuration. In some embodiments, the surface area that comprises the return electrode 4195 which makes contact with the electrically conductive material is optimally chosen to minimize the current density with the objective of minimizing the heating on the surface of the return electrode 4195. In other embodiments, the conductor cable 4120 comprises more than two conductors to accommodate other attributes of the device. In other embodiments, the SPDT switch 4145 may be positioned on the conductor cabling 4120 between the connector 4100 and the handle 4140; in such embodiments the electrode conductors 4176 and 4177 leading to the electrodes 4185 and 4190 may extend through or around the shaft 4175 and handle 4140 and may comprise at least part of conductor cabling 4120. In other embodiments, the shaft 4175 may contain more than 3 conductors. In alternative embodiments, each conductor within the conductor cable 4120 may be contained in its own cabling. In an alternative embodiment, the switch 4145 may have more than 2 positions.

An alternative embodiment of the bipolar electrical wiring for the TDM may include a circuit that modulates the energy delivered at the electrodes similar to that described in FIG. 15d.

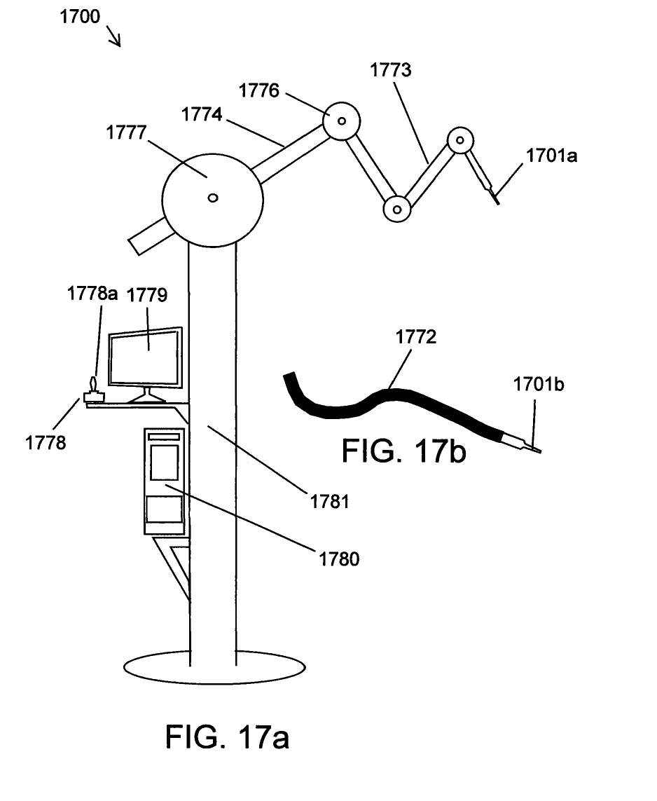

An embodiment of a system 1700 for performing robotic surgery using a modular TD is depicted in FIG. 17a. System 1700 may comprise a tissue dissecting and modifying wand (TDM) 1701a. TDM 1701a may comprise a tissue dissecting and modifying wand (TDM) that may, as described elsewhere herein, comprise a plurality of protrusions with one or more recessions positioned therebetween. TDM 1701a may be coupled with one or more robotic surgery components, such as a surgical arm.

In some embodiments, TDM 1701a may comprise a shaft, a tip, and/or a handle, as described elsewhere in this disclosure. In such embodiments, TDM 1701a may be selectively coupled to a robotic arm such that the TDM 1701a can either be used by hand, or coupled with one or more robotic surgery components to allow a surgeon to perform a surgical procedure with the TDM 1701a remotely and/or indirectly. In other embodiments, the TDM may be configured to be integrally coupled with, or otherwise non-selectively coupled with, one or more robotic surgery components. In such embodiments, it may not be necessary to configure the TDM 1701a with a handle and/or shaft. In other words, in some embodiments, the TDM 1701a may comprise only a tip.

In some embodiments, the robotic surgery system 1700 may comprise one or more motors, such as a screw-drive motor, gear motor, hydraulic motors, etc. In some embodiments, the robotic surgery system 1700 may comprise worm gearheads, video cameras, motor control circuits, monitors, remote control devices, illumination sources, tactile interface, etc. In the embodiment depicted in FIG. 17a, TDM 1700 comprises a TDM tip 1701a that is positioned at the end of a robotic arm. This robotic arm comprises a plurality of arm segments 1773 with corresponding joints 1776 positioned therebetween. A primary joint 1777 may be positioned to support and articulate together each of the arm segments 1773 and smaller joints 1776. Primary joint has a primary arm segment 1774 that extends therefrom. Finer movements of the robotic arm may then be accomplished using one or more of the smaller joints 1776.

A stand 1781 may also be provided to support the various robotic arms. In some embodiments, stand 1781 may also be configured to support a monitor 1779 and/or other display, input, or control components, such as a control element 1778. In some embodiments, control element 1778 may comprise a hand control toggle 1778a. In other embodiments, control element 1778 may comprise a keyboard, mouse, touchscreen display, virtual reality system, control pad, or the like. Monitor 1779 and/or control element 1778 may be communicatively coupled with a central processing unit 1780.

Central processing unit 1780 may comprise, for example, one or more microprocessors and/or other electronic components, such as data connectivity elements, memory, non-transitory computer readable media, etc. In some embodiments, central processing unit 1780 may comprise a general-purpose computer. Central processing unit 1780 may further comprise a machine-readable storage device, such as non-volatile memory, static RAM, dynamic RAM, ROM, CD-ROM, disk, tape, magnetic storage, optical storage, flash memory, or another machine-readable storage medium.

FIG. 17b illustrates an alternative embodiment of a robotic arm 1772 that may be used with system 1700. Robotic arm 1772 comprises an endoscopic snake-like robotic arm 1772 and also comprises a TDM 1701b positioned at its distal end. As with the embodiment of FIG. 7a, TDM 1701b may be selectively coupled to robotic arm 1772 or, alternatively, may be integrally or otherwise non-selectively coupled to robotic arm 1772. Further details regarding robotic surgery components that may be useful in connection with the various embodiments disclosed herein may be found in the following U.S. Pat. Nos. each of which is hereby incorporated by reference in its entirety: U.S. Pat. No. 4,259,876 titled Mechanical Arm, U.S. Pat. No. 4,221,997 titled Articulated Robot Arm and Method Of Moving Same, U.S. Pat. No. 4,462,748 titled Industrial Robot, U.S. Pat. No. 4,494,417 titled Flexible Arm, Particularly a Robot Arm, U.S. Pat. No. 4,631,689 titled Multi-Joint Arm Robot Apparatus, U.S. Pat. No. 4,806,066 titled Robotic Arm, U.S. Pat. No. 5,791,231 titled Surgical Robotic System and Hydraulic Actuator Therefore, U.S. Pat. No. 7,199,545 titled Robot For Surgical Applications, U.S. Pat. No. 7,316,681 titled Articulated Surgical Instrument For Performing Minimally Invasive Surgery With Enhanced Dexterity and Sensitivity, U.S. Pat. No. 8,182,418 titled Systems and Methods for Articulating An Elongate Body, U.S. Pat. No. 8,224,485 titled Snaking Robotic Arm With Movable Shapers.

Any of the embodiments of TD discussed herein including, but not limited to, the embodiments discussed with FIGS. 1-30 may be used in conjunction with one or more of the robotic surgery elements disclosed in connection with FIGS. 17a-b.

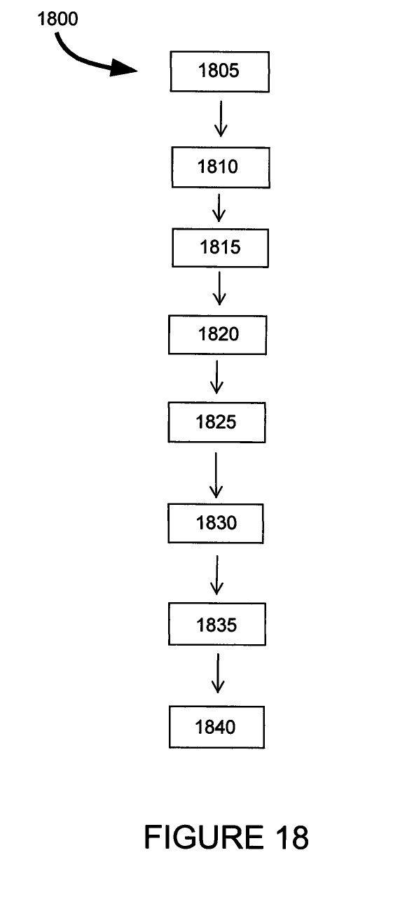

FIG. 18 depicts a flow chart of an implementation of an energy emission—sensor feedback loop 1800 according to this disclosure. Step 1805 may comprise: setting one or more temperatures (a desired maximal temperature threshold, or a range). In other implementations one or more such temperatures may be preset by the manufacturer. Step 1810 may comprise setting one or more energy levels to lysing area and/or energy windows (a desired maximal energy threshold, or a range). In other implementations energy levels may be preset by the manufacturer. Step 1815 may comprise passing the TD through or by the target tissue area. Step 1820 may comprise applying electrosurgical energy at lysing elements. Step 1825 may comprise applying energy at the energy window(s). In some implementations energy may be only applied at the lysing elements. In other implementations, energy may only be applied to the energy window(s). Step 1830 may comprise gathering sensor data, such as temperature data. Step 1835 may comprise comparing sensor data to one or more set temperature levels. Step 1840 may comprise, if the sensed temperature exceeds the threshold, reducing the amount of energy delivered through the lysing elements and/or the energy window(s).

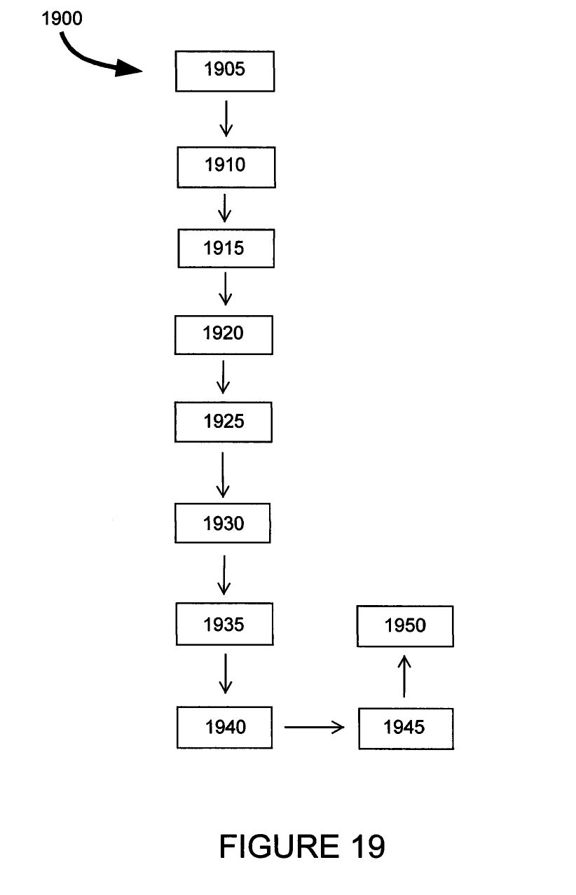

FIG. 19 illustrates an implementation of a method 1900 according to this disclosure for accessing an organ with the assistance of a TD. In some implementations, surgeon(s) may need to access tissue and/or an organ to repair or to treat it. In some implementations, the skin surrounding the anticipated entrance wound for the surgical area may be cleansed by, for example, with isopropyl alcohol (degreaser) followed by a germicide, for example, chlorhexidine scrub. Then, a local anesthetic may be applied (such as by injecting) 1% lidocaine+1:100,000 adrenaline to the skin.

Step 1905 may comprise forming an incision. In some implementations, step 1905 may comprise making the incision using the same surgical tool, such as a TD, used to perform every step in method 1900. In other implementations, step 1905 may be performed by another surgical instrument such as a scalpel. In some implementations, the incision may be minimally invasive. In other implementations, the incision may be open and/or invasive Step 1905 may be performed with, for example, a #15 Bard-Parker™ Scalpel. This incision may be deepened by scalpel, scissors or other surgical instrument to enter the desired body structure or cavity. For larger approaches, such as open abdominal surgery or trauma surgery step 1905 may comprise the initial skin opening or body cavity opening steps of such a procedure. In some implementations, step 1905 may comprise making the skin incision using the lysing elements of the TD. Step 1910 may comprise: applying one or more fluids to other tissue layers. In some implementations, step 1910 may comprise applying fluids to the target tissue(s). In some implementations, step 1910 may comprise applying fluids to the tissues to be traversed en route to the target tissue, in addition to, or as an alternative to applying fluids directly to the target tissue(s). In some implementations, the fluid(s) may comprise water. In some implementations, the fluid(s) may comprise an ionic fluid, such as a saline solution. The fluid(s) may be applied to the tissue via, for example, injection, or TDM fluid port or via a separate cannula or catheter or via pouring or via spray. In some implementations, the fluid(s) may comprise an ionic fluid and an anesthetic, such as a tumescent anesthesia. Non-ionic fluids may be used in other implementations; such fluids may become more ionic by diffusion of some of the patients' ions present in the surgical field. In some implementations step 1910 may comprise applying one or more fluids that serve as an ionic fluid, and/or an anesthetic, and/or adrenaline. In some such implementations, the fluid(s) may comprise a Klein Formula. In some implementations, the Klein formula and amount used may be about 100 cc of Klein Formula with saline, 0.1% lidocaine, epinephrine 1:1,000,000, and NaHCO3 @5 meq/L of saline).

Step 1915 may comprise: passing the TD and/or TDM through the various layers of tissue to create a path to a target organ. In some implementations, creating a path to a target organ or other target tissue may comprise creating a path from the incision to the target organ or other target tissue and/or creating a path around the target organ or other target tissue to allow for access to other regions of the target organ or other target tissue. In some implementations step 1915 may further comprise activating the lysing elements and/or energy window to reduce bleeding or tissues traversed on the way to the target organ. In some implementations, the lysing elements and/or energy window may be used to induce fibrosis along the path, including along a path that may traverse the perimeter of the target organ/tissue. In some implementations, step, may comprise visualizing the anticipated path for the TD using for example an endoscope, a fiberoptic or camera, an ITOMSensor, RFID and/or other antenna. The fiberoptic, camera and/or RFID tag may be positioned on the TD. In some implementations, such a device or devices may be positioned on the TD. In other implementations such a device or devices may be separate from the TD. ‘ITOMSensor step’ 1920 may comprise tracking the movement of the surgical tool with respect to an internal body tissue. In some implementations step 1920 may comprise use of image processing technology such as described herein (such as discussed before in FIGS. 2 & 14. In some implementations, heat may be produced or energy may otherwise be released in the tissues through which the TD is passed. In some implementations, heating portions of the tissues the TD passes by may be undesirable. As such, in some implementations, undesirable heating of such layers may be mitigated by applying a cooling step antecedent and or concurrent with energy delivery with the TD. Such steps may comprise use of one or more cooling fluids delivered via the TD or one or more separate catheters &/or cannulas &/or endoscopes. Such cooling mechanism(s) may comprise for example, a closed water bag. Such a bag may be at a temperature of less than 37° C. In some implementations, cooling objects such as fluid or gel filled bags may be used that may range in temperature between about 1° C. to about 20° C. In some such implementations, the fluid or gel may be about 15° C. Other cooling mechanisms may comprise a dynamic cooling system wherein a cool liquid or gel is actively pumped into or through a contact cooling object. Step 1925 may comprise identifying important blood vessels, nerves, ducts, organs or other anatomy in the area surrounding the target tissue. Step 1930 may comprise spot coagulating one or more blood vessels, for example using spot coagulator 662 from previous FIG. 6a. Step 1930 may be repeated as needed throughout method 1900 to coagulate blood vessels as they are exposed and/or traumatized during a surgical procedure. Step 1935 may comprise: adding additional fluids of the types previously described to the target and/or surrounding tissues via the TD port(s) or via one or more separate catheters &/or cannulas &/or endoscopes. Step 1940 may comprise: expanding one or more regions of the path to the target tissue. In some implementations, step 1940 may comprise expanding one or more path(s) from the incision to the target tissue. In some implementations, step 1940 may comprise expanding a region around the target tissue such as for example, via a fanning motion. In some implementations, one or more of the other steps described herein using the TD may also be performed with a fanning motion. In implementations using TDs with axially oriented protrusions, such a fanning motion may comprise a to and fro spokewheel pattern. In implementations using TDs with non-axially oriented protrusions, such a fanning motion may comprise a side-to-side fanning motion; one example of a fanning motion using a TD having at least one nonaxially oriented protrusion may comprise a ‘windshield wiper’ motion. In some implementations, step 1940 may further comprise activating the energy to the TD for example the energy to the lysing elements and/or one or more energy windows. In some implementations, step 1945 may comprise spot coagulating one or more additional blood vessels that may be warranted to spot coagulate at this moment, for example using spot coagulator 662 from previous FIG. 6a. In some implementations, step 1945 may comprise repeating spot coagulation when more bleeding vessels are encountered and/or observed. In some implementations achieving hemostasis may be accomplished by cautery, electrifying, ligating, or chemical methods. In some implementations, the lysing element and/or the energy window can be used to achieve the hemostasis. In some implementations, one or more other devices and/or suture and/or surgeon's hands may be used to achieve hemostasis for larger vessels. Step 1950 may comprise: removing the TD with power off and suturing the wound in the standard fashion. In some implementations, the tissues traversed may require closure by suturing and/or stapling. In some implementations, organs and/or organ systems that the TD may be useful to access may include but not limited to muscle, and/or parotid, and/or salivary gland, and/or thyroid, and/or lung, and/or heart, and/or gastrointestinal, and/or liver, and/or pancreas, and/or spleen, and/or gallbladder, and/or kidney, and/or adrenal, and/or prostate, and/or ovary, and/or uterus, and/or bladder, and/or vascular, and/or lymph nodes and/or skeleton, and/or lung.

Any of the embodiments of TDM and/or TD discussed herein including, but not limited to, the embodiments discussed with FIGS. 1-29, etc. may be used in conjunction with one or more of the steps disclosed in connection with FIG. 19.

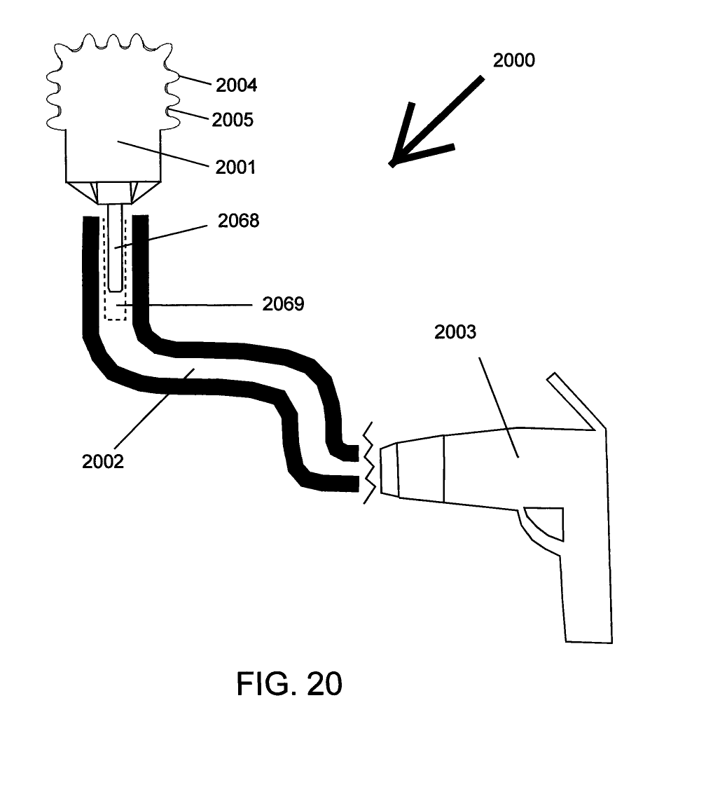

FIG. 20 depicts an embodiment of a modular TD 2000 comprising a tip 2001, a shaft 2002, and an endoscope handle 2003. In some embodiments, shaft 2002 comprises a flexible shaft. Tip 2001 is modular in that it is removable from flexible shaft 2002. More particularly, tip 2001 comprises a means for removably coupling the tip with a shaft at 2068. In the depicted embodiment, this coupling means comprises a tip plug 2068. In some embodiments, tip plug 2068 may be threaded to facilitate a secure coupling between modular tip 2001 and shaft 2002. However, in other embodiments, the coupling means may comprise a recess configured to receive a plug formed on the shaft. In still other embodiments, the coupling means may comprise a snap-fit coupling, a friction fit coupling, a bayonet clip, etc.

In the depicted embodiment, tip plug 2068 is configured to be received within a corresponding recess 2069 formed within shaft 2002. In some embodiments, tip plug 2068 may be configured to electrically couple tip 2001 with shaft 2002. In this manner, in embodiments comprising, for example, lysing elements, electricity from a power source may be transmitted through the coupling between plug 2068 and recess 2069 to allow for energizing the lysing elements. Other embodiments may be configured to transfer additional electricity, data, or materials through such coupling. For example, in embodiments comprising one or more sensors on tip 2001, a signal from such sensor(s) may be transmitted through shaft 2002 by way of the coupling means 2068.

In some embodiments, tip 2001 may be disposable as well, such that a surgeon can place an appropriate tip on the shaft and remove and dispose of the tip after surgery. Alternatively or additionally, a plurality of different tips may be provided, each of which may be disposable, or may be configured for sterilization and re-use, and an appropriate tip may be selected as needed for a particular surgery.

In the depicted embodiment, tip 2001 comprises a plurality of protrusions 2004, some of which are non-axial, and a plurality of recessions 2005 positioned therebetween, as described above. In some embodiments a tip comprising only axial protrusions may be swapped for tip 2001 as desired to suit a particular surgical procedure.

Any of the embodiments of TD discussed herein including, but not limited to, the embodiments discussed with FIGS. 1-30, etc. may be used in conjunction with one or more of the steps disclosed in connection with FIG. 20.

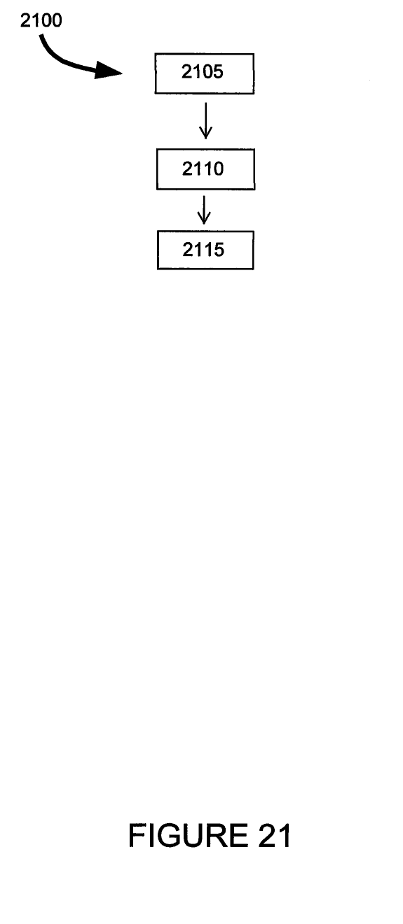

FIG. 21 depicts a flow chart of an implementation of a method 2100 for separating and/or modifying tissue using a TD. In this particular implementation, the use of combined data from the tissue dissecting and modifying wand generated from at least the temperature sensor and the ITOMSensor(s) may be used to provide suitable feedback to a user during treatment. In some implementations, the TD may comprise a tip comprising a plurality of protrusions. One or more lysing elements may be positioned between at least two adjacent protrusions among the plurality of protrusions. A temperature sensor may be positioned on the TD. The temperature sensor may be configured to sense a temperature of at least one of tissue and fluid adjacent to the TD during an operation. The fluid of which a temperature reading is taken may comprise, for example, fluid from adjacent tissue(s) and/or fluid introduced during the procedure by way of the TD and/or another device or procedure. The TD may also comprise an ITOMSensor positioned on the TD. In some implementations, the ITOMSensor(s) may be positioned on the tip and/or distal end of the shaft. The ITOMSensor(s) may be configured to provide speed of motion data regarding the TD, during an operation or procedure. Although method 2100 is shown in the figure beginning with step 2105, it should be understood that any of the preliminary steps described above in connection with other implementations may be performed in method 2100 as well. For example, one or more of steps (1905-1940) from method 1900 may be performed in method 2100 if desired. Similarly, one or more other steps of any of the other implementations described herein may also be included in the method depicted in FIG. 21. In some implementations, step 2105 may comprise: receiving data from the TD temperature sensor. Step 2110 may comprise receiving data from the ITOMSensor. Step 2115 may comprise combining the data generated from at least the temperature sensor and the ITOMSensor (s). In some implementations, the data from the temperature sensor and the ITOMSensor (s) may be combined before it is received. In other words, a step of “receiving combined data from the tissue dissecting and modifying wand generated from at least the temperature sensor and the ITOMSensor (s) may comprise receiving precombined data (data from the temperature sensor and the ITOMSensor (s) that was combined before it was received) or, alternatively, may comprise separately receiving temperature data and ITOMSensor (s) data that may be combined to allow for one or more particular features or functionalities. The combined data may be used to allow a surgeon or other user to determine one or more regions within a patient's body that have been adequately treated using the TD. For example, in some implementations, the combined data may allow a user to determine whether one or more regions within a patient's body have been sufficiently treated, and/or to modulate one or more aspects of energy output to maintain desired treatment parameter(s). In some implementations, determining whether one or more regions within a patient's body have been sufficiently treated may comprise visualizing one or more regions within a patient's body during treatment. This may be accomplished, for example, by creating an image corresponding with one or more regions of a patient's body. In some implementations, for TD comprising lysing elements but lacking an energy window, the combined data may be processed and/or electromagnetic lysing element output parameters automatically modulated. In some implementations, for TD comprising lysing elements and an energy window, the combined data may be processed &/or electromagnetic lysing element output parameters automatically modulated &/or energy window output parameters automatically modulated. For example, in implementations wherein an LASER energy window is present the following may be modulated: LASER power &/or wavelength &/or beam intensity &/or pulse duration &/or pulse rate &/or emission rate, For example, in implementations wherein an RF/electrosurgical energy window is present the following may be modulated: power &/or waveform &/or voltage &/or amperage &/or pulse duration &/or pulse rate &/or duty cycle. In some implementations, such adequately treated regions may correspond with regions comprising tissue that has reached a predetermined threshold temperature.

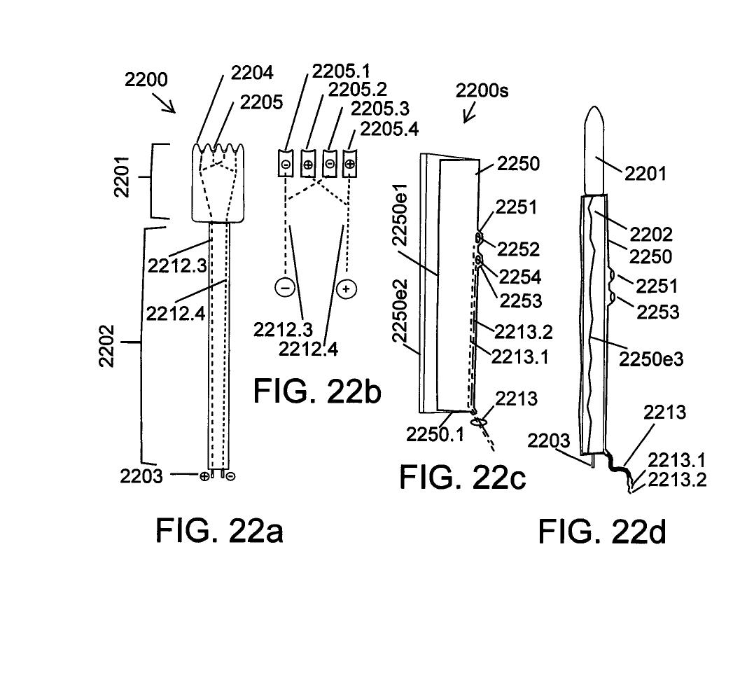

FIG. 22a depicts an embodiment of a modular bipolar Tissue Dissector (modular bipolar TD) 2200 comprising a modular bipolar TD tip 2201, a modular bipolar TD shaft 2202 and a modular bipolar TD plug 2203. In the depicted embodiment, protrusions 2204 and lysing elements 2305 are oriented an axial direction. An external power cord may bring RF electrosurgical energy from an electrosurgical generator to modular bipolar TD plug 2203 (which is electrically connected) to modular bipolar TD shaft 2202 and thus to electrically conductive lysing elements 2205, mounted in the recessions in between protrusions, such as protrusions 2204. In some embodiments, the protrusions may comprise bulbous protrusions. The tip shown in this embodiment has five relative protrusions 2204, four lysing elements 2205 and four relative recessions pointing along the main axis of the modular bipolar TD. In other embodiments, the modular bipolar TD tip may have one or more non-axial protrusions and one or more non-axial relative recessions. In some embodiments, the tip may have between 3 and 100 axial and/or non-axial protrusions and/or relative recessions. It should be understood that the number of protrusions need not match the number of lysing elements or recessions. In some embodiments, lysing elements may be located at the termini of conductive elements. In some embodiments, lysing elements may also be made partially or completely of a cermet material. In an embodiment, the modular bipolar TD tip 2201 may measure about 15 mm in width, about 18 mm in length and about 5 mm in maximal thickness; the modular bipolar TD shaft 2202 may measure about 6 mm in diameter and/or about 60 mm in length; the overall modular bipolar TD 2200 may measure about 15 mm in width, 80 mm in length and about 6 mm in maximal thickness. Embodiments are contemplated wherein sizes of about one-fifth to about five times these dimensions may have possible uses. It is also contemplated, for example in some veterinary embodiments; tip sizes of about one-tenth to 20 times the aforementioned dimensions may also have possible uses. In some embodiments, wherein electrical insulation and/or polymeric insulating coating is present on such parts, for example modular bipolar TD shaft 2202, such insulation may measure about 0.5 mm in thickness; in some contemplated embodiments, the insulation thickness may range from 0.1 mm to 3 mm. In the depicted embodiment, electrical leads 2212.3 and 2212.4 may course from plug 2203 via shaft 2202 to energize various lysing elements located in bipolar TD tip 2201. In some embodiments leads may comprise wires and/or conductive conduits. In other contemplated embodiments leads may be printed on via techniques that may be akin to those of MicroPen™ discussed elsewhere in this application.

FIG. 22b is an upper plan view of the four lysing elements of the embodiment depicted in FIG. 22a. In this embodiment, lysing elements may be comprised of even numbers of oppositely charged (when activated) individual lysing elements 2205.1, 2205.2, 2205.3 and 2205.4. In this embodiment, individual bipolar TD lysing elements 2205.1, 2205.2, 2205.3 and/or 2205.4 may comprise surgical grade stainless steel positioned within all and/or a portion of one or more pieces of ceramic and/or other thermally resistant, non-conductive housing. In some embodiments, one or more individual lysing elements may comprise electroconductive materials including but not limited to cermets, steel, nickel, alloys, palladium, gold, tungsten, titanium, silver, copper, and/or platinum. In the depicted embodiment, the lysing elements may measure about 2 mm in width, 5 mm in length and about 0.5 mm in thickness. In the depicted embodiment, the axial lysing elements are concave and crescentic in shape. However, in other contemplated embodiments lysing elements may comprise straight and/or convex and/or a variety of shapes. In other contemplated embodiments, lysing elements may vary from about one-fifth to about 20 times the aforementioned sizes and be of a variety of shapes. In the depicted embodiment, similarly charged individual lysing elements 2205.1, 2205.3 are energized by ‘negative’ lead 2212.3; similarly charged individual lysing elements 2205.2, 2205.4 are energized by ‘positive’ lead 2212.4.

In some contemplated embodiments, lysing elements carrying similar charges need not be individual but may be joined and/or continuous with one or more lysing elements stamped and/or made from a common piece. In other embodiments, lysing elements may be formed and/or joined from one or more conductive parts.

In some contemplated embodiments there need not be equal numbers of oppositely signed and/or charged individual lysing elements, for example, there may be 3 positive and 2 negative individual lysing elements. Uniformity of flux on activation may be achieved by modifying the size and/or position of lysing elements with respect to each other among other methods known in the art.

The relative static permittivity of some ceramics may range from about 5 to 10; this may cause some leakage of current in an undesirable path between closely approximated opposing electrodes during activation. Use of other materials, for example, those having over of relative static permittivities over 5 may undesirably alter the resultant plasma field. The relative static permittivity of the intervening materials housing the opposing electrodes may be enhanced by coating and/or surrounding and/or injection molding thermoresistant polymers of a low relative static permittivity into the housing and/or around one or more portions of bipolar lysing elements 2205 to reduce the effective static permittivity of the tip. In an embodiment, the thermoresistant polymer of low relative static permittivity 2.1 may be polytetrafluoroethylene. In other contemplated embodiments, thermoresistant polymers may include polyether etherketone (@3.3) and/or polysulfone (@3.1) and the like may be useful.

In the depicted embodiments, the electrical insulator comprises polytetrafluoroethylene. In other contemplated embodiments, the electrical insulator may comprise an electrically nonconductive polymer with a high melting temperature. In some embodiments, the nonconductive polymer may comprise for example, polyether etherketone and/or polysulfone, etc. In other contemplated embodiments, the electrical insulator may comprise an electrically nonconductive and/or thermally nonconductive polymer.

FIG. 22c is a side view of a bipolar circuit activating foot-pedal bypass (BCAFPB) 2200s, which may be coupled with a surgical tool such as modular bipolar TD 2200 as depicted in FIG. 22d. In the embodiment depicted in FIG. 22c the BCAFPB comprises a sticker. In some embodiments, the sticker may be configured to wrap around the surgical tool. BCAFPB 2200s is one example of a Means for Allowing Hand Control of a Footpedal Activated Surgical Tool (MAHCFAST). In some embodiments comprising a MAHCFAST, the foot control may be disabled in favor of the hand control. In other embodiments, the MAHCFAST may facilitate hand control without disabling foot control. Sticker 2200s may comprise an adhesive backed wrapper or the like, pressure activated on-off switch 2252, casing 2251 for switch 2252, casing 2253 for a second switch and/or second switch 2254 (which may be used to control another function of the electrosurgical generator such as increasing or decreasing the power and/or other settings), electric lead 2213.1 (which connects to switch 2252), and electric lead 2213.2 (which may connect to second switch 2254) and opening 2250.1. Electric leads 2213.1 & 2213.2 may be bound together in conduit 2213. Sticker 2250 may comprise silicone and/or plastic and/or polyethylene and/or polyurethane and/or polypropylene and/or any other sterilizable and/or biocompatible polymer, etc. In the depicted embodiment, sticker 2250 comprises edges 2250e1 & 2250e2 & a sterile pressure sensitive acrylic adhesive. In some embodiments, sticker 2250 may be stretchable and/or elastic in order to fit more tightly over contours in the intended bipolar surgical instrument. In other contemplated embodiments, sticker 2250 may comprise pressure sensitive adhesives including but not limited to those based on: silicone, polyester, polyether & polyurethane. In other contemplated embodiments, sticker 2250 may be rigid and may require pleating along some of its axis in order to take up some extra slack following adhesion. Once applied to a surgical tool, edges 2250e1 and 2250e2 may for some surgical tools overlap; for other surgical tools the edges need not overlap.

A large portion of bipolar hand-held electrosurgical instruments are foot-pedal activated. Some surgeons may complain about having their feet restricted to the zone around the foot-pedal and/or about striking the wrong plate of the foot-pedal and/or having to find the foot-pedal during a lengthy procedure. Foot-pedal activation may also be disadvantageous for certain procedures since significant damage to vital organ(s) may happen in a fraction of a second if electrosurgical energy is applied erroneously. It may therefore be desirable for surgeons to have a method of hand control available over the foot-pedal circuitry; especially a control which is located adjacent and/or affixed to the bipolar instrument. It may be convenient to have the quicker hand response time available in a removable sheath that may accommodate and/or ‘fit’ over a one or more hand held bipolar instruments. Reported differences between eye-hand and eye-foot response times (0.28 sec for hand versus 0.45 sec for foot) may be in large part due to the extra distance nerve impulses must travel to the foot. (reference: J Amer Optom Asn, 2000; 71(12) 775-780). Metal bands and/or a glue seal and/or a tie off and/or another type of fastener may be located at any positions throughout 2250.1 to prevent continued movement of shaft 2202 within the sticker.

It is contemplated that BCAFPB may be customized to a particular surgical tool by way of shape and/or size and further that a variety of shapes and/or sizes may be provided, each of which may be designed to work in conjunction with one or more surgical bipolar tools. It is also contemplated that some BCAFPB 2250 may be cut to size prior to or during the operation. It is also contemplated that many alternative components and features may be provided for example, some embodiments may comprise in place of or in addition to pressure activated on off switch 2252 and or 2254 one or more other types of control elements such as flip switches, optically activated buttons, electrostatically operated buttons, and the like. In other contemplated embodiments the means for allowing hand control of a footpedal activated surgical tool may instead comprise a sheath, a clip, a ring, or the like that may be coupled with a bipolar surgical tool by any available means such as an adhesive, welding, hook and loop fastener and the like.

FIG. 22d is side view depicting an embodiment of a sticker 2200s placed over bipolar TD shaft 2202. In the depicted embodiment tip 2201 is fully exposed after application of sticker 2200s, however in other embodiments, sticker 2200s may be configured to also partially cover tip 2201. Similarly, although in the depicted embodiment sticker 2200s fully covers shaft 2202, in other embodiments, sticker 2200s may only cover a portion of shaft 2202 and/or another portion of surgical tool 2200. In the embodiment depicted in FIG. 22d, edges 2250e1 & 2250e2 overlap as shown at 2250e3, the amount of overlap may vary depending upon the surgical tool.

As of the year 2000, the bipolar mode had traditionally been used primarily for coagulation, (reference: “The Biomedical Engineering Handbook, Electrosurgical Devices” J Eggleston, W Maltzahn, Ch 81, CRC Press 2000). However, more recent modifications to bipolar electrosurgical outputs may have facilitated the use of bipolar cutting instruments (reference: ValleyLab, Hotline, vol. 4, issue 4 pg. 1), examples of such outputs may include Macrobipolar settings (Reference: ValleyLab ForceTriad Users Guide 2006, chapter/sections: 9-13, 9-16, 9-24).

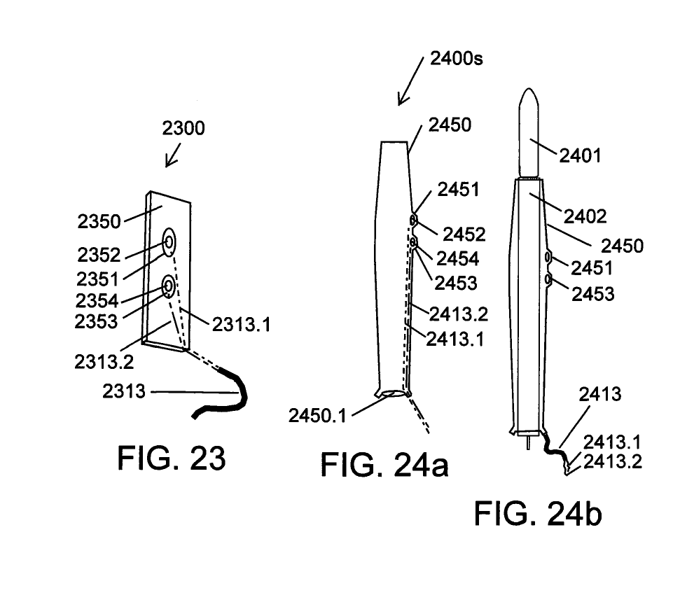

FIG. 23 depicts an embodiment of a BCAFPB comprising an adhesive backed pad. Adhesive backed pad 2300 should also be considered an example of a sticker. FIG. 23 depicts an embodiment that differs from the embodiment depicted in FIGS. 22c,d in that it comprises a smaller adhesive pad 2300 and FIG. 22 comprises a larger sticker. Each of the other elements depicted in FIG. 23 may be identical to the corresponding elements shown in FIGS. 22c,d and are referenced by like numerals (numbers higher by 100). Pad 2300 may comprise an upper surface 2350. An opposite lower surface (not shown) may comprise an adhesive for coupling pad 2300 with a surgical tool. One or more buttons or button encasements such as buttons 2352 & 2354 and encasements 2351 & 2353 may be positioned on upper surface 2350. In alternative embodiments, one or more buttons 2352 & 2354 may be replaced by alternative control elements as discussed above. Electrical lead 2313.1 is electrically coupled with button 2352. Similarly, electrical lead 2313.2 is electrically coupled with button 2354. Leads 2313.1 & 2313.2 may be bound together in conduit 2313.

FIGS. 24a,b depict an embodiment that differs from the embodiment depicted in FIGS. 22c,d in that FIGS. 24a,b comprises a sheath 2400 and FIGS. 22c,d comprises a sticker. Each of the other elements depicted in FIGS. 24a,b may be identical to the corresponding elements shown in FIGS. 22c,d and are referenced by like numerals (numbers higher by 200). For example, in FIGS. 24a,b sheath 2400s comprises a tubular body 2450 comprising at least one open end 2450.1. Sheath 2400s also comprises casing 2451, switch 2452, second casing 2453, second switch 2454, corresponding surgical leads 2413.1 & 2413.2. FIG. 24b depicts sheath 2400s after it has been positioned over shaft 2402. In the depicted embodiment, sheath 2400s is configured to be put in to position such that tip 2401 is fully exposed. However in other embodiments sheaths may be configured to only partially expose a tip of a surgical tool.

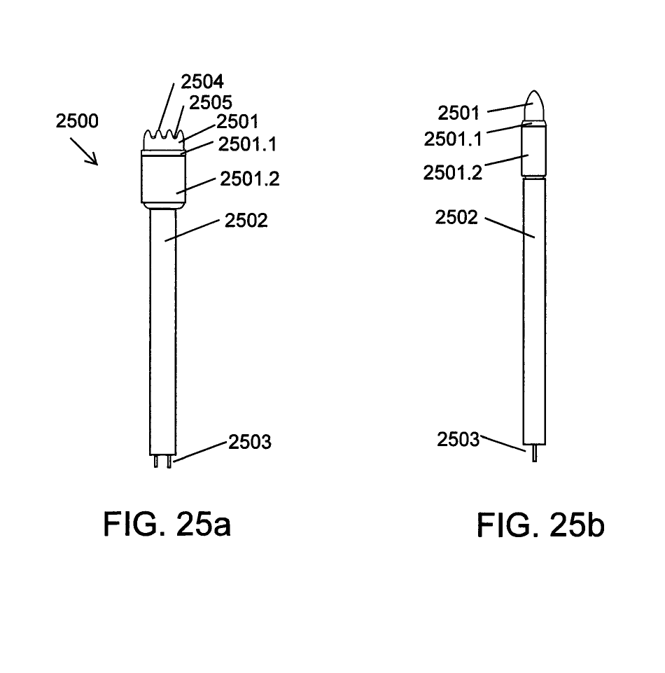

FIG. 25a depicts an embodiment of a modular common intermediate configuration Tissue Dissector (modular CiCTD) 2500 comprising a modular CiCTD tip 2501, a modular CiCTD shaft 2502 and a modular CiCTD plug 2503. Embodiments wherein both electrodes are located on the same probe and/or device, but the return electrode is larger than the active electrode may be termed by some surgeons as ‘common intermediate configuration.’

In the depicted embodiment, protrusions 2504 and lysing elements 2505 are oriented an axial direction and return electrode 2501.2 may be located on the tip but electrically isolated from each of the lysing elements. An external power cord may bring RF electrosurgical energy from an electrosurgical generator to modular CiCTD plug 2503 (which is electrically connected) to modular CiCTD shaft 2502 and thus to electrically conductive lysing elements 2505, mounted in the recessions in between protrusions, such as protrusions 2504. In some embodiments, the protrusions may comprise bulbous protrusions. The tip shown in this embodiment has four relative protrusions 2504, three lysing elements 2505 and three relative recessions pointing along the main axis of the CiCTD. In other embodiments, the modular CiCTD tip may have one or more non-axial protrusions and one or more non-axial relative recessions. In some embodiments, the tip may have between 3 and 100 axial and/or non-axial protrusions and/or relative recessions. It should be understood that the number of protrusions need not match the number of lysing elements or recessions. In some embodiments, lysing elements may be located at the termini of conductive elements. In some embodiments, lysing elements may also be made partially or completely of a cermet material. In some embodiments, return electrodes may also be made partially or completely of a cermet material. In an embodiment, the modular CiCTD tip 2501 may measure about 12 mm in width, about 15 mm in length and about 5 mm in maximal thickness; the modular CiCTD shaft 2502 may measure about 6 mm in diameter and/or about 60 mm in length; the overall modular CiCTD 2500 may measure about 12 mm in width, 75 mm in length and about 6 mm in maximal thickness. Embodiments are contemplated wherein sizes of about one-fifth to about five times these dimensions may have possible uses. It is also contemplated, for example in some veterinary embodiments; tip sizes of about one-tenth to 20 times the aforementioned dimensions may also have possible uses. In some embodiments, wherein electrical insulation and/or polymeric insulating coating is present on such parts, for example modular CiCTD shaft 2502, such insulation may measure about 0.5 mm in thickness; in some contemplated embodiments, the insulation thickness may range from 0.1 mm to 3 mm.

In upper plan view of the depicted embodiment of FIG. 25a, the visible CiCTD lysing element 2505 portions may be part of a larger piece of active electrode of surgical grade stainless steel lying within all and/or a portion of one or more pieces of ceramic and/or other thermally resistant, non-conductive housing. In some embodiments, one or more pieces of active electrode may be comprised of electroconductive materials including but not limited to cermets, steel, nickel, alloys, palladium, gold, tungsten, titanium, silver, copper, and/or platinum. In the depicted embodiment, the piece of active electrode comprises a plate of about 0.5 mm in thickness. In other embodiments, lysing elements may be formed from one or more conductive parts. The relative static permittivity of some ceramics may range from about 5 to 10; this may cause some leakage of current in an undesirable path during activation of active electrode. In some embodiments, wherein a return electrode is positioned on the exterior of CiCTD tip 2501, the relative static permittivity of the intervening materials housing the active electrode (and as may be the case in the CiCTD tip) may be enhanced by coating and/or surrounding and/or injection molding thermoresistant polymers &/or thermoresistant materials of a low relative static permittivity into the housing and/or around one or more portions of an active electrode piece comprising, at least in part, CiCTD lysing element 2505. In an embodiment, the thermoresistant polymer of low relative static permittivity 2.1 is polytetrafluoroethylene. In other contemplated embodiments, thermoresistant polymers may comprise polyether etherketone (@3.3) and/or polysulfone (@3.1) and the like which may be useful. In other contemplated embodiments, thermoresistant materials may comprise polyether etherketone &/or PFTE with hollow glass microspheres.

In the depicted embodiments of FIGS. 25a & b, CiCTD tip return electrode 2501.2 may be located on one or more sides and/or edges of modular CiCTD tip 2501 In the depicted embodiment, CiCTD tip return electrode 2501.2 is an external band comprised of a biocompatible conductive metal, for example, surgical stainless steel, tightly fastened to the modular CiCTD tip 2501. In some embodiments, CiCTD return electrode is located on the external aspects of tip 2501 and/or shaft 2502. In some embodiments, CiCTD tip return electrode 2501.2 may be comprised of electroconductive materials including but not limited to cermets, steel, nickel, alloys, palladium, gold, tungsten, titanium, silver, copper, and platinum. In some embodiments, modular CiCTD tip may comprise materials that are electrically non-conductive and/or of low thermal conductivity; such materials may comprise, for example, porcelain, ceramics, glass-ceramics, plastics, varieties of polytetrafluoroethylene, carbon, graphite, and/or graphite-fiberglass composites, etc. In some embodiments, the modular TD tip may be constructed of a support matrix of an insulating material (e.g., ceramic or glass material such as alumina, zirconia). In some embodiments, the modular TD tip may comprise cermets.

In other contemplated embodiments, the modular TD tip may comprise polyether etherketone &/or polytetrafluoroethylene with hollow glass microspheres.

In some embodiments, the CiCTD tip return electrode 2501.2 may be in the shape of a plate and/or plane and/or may be made of any metal or alloy that does not melt under operating conditions or give-off toxic residua. In the depicted embodiment, the CiCTD tip return electrode 2501.2 comprises a band of surgical grade stainless steel comprising a thickness of 0.5 mm and a thickness of about 1 cm which may be wrapped around CICTD tip 2501. Embodiments are contemplated of the CiCTD tip return electrode 2501.2 wherein sizes of about one-fifth to about five times these dimensions may have possible uses. It is also contemplated, for example in some veterinary embodiments; tip sizes of about one-tenth to 20 times the aforementioned dimensions may also have possible uses. In some embodiments, the CICTD tip has curviform edges thus reducing sharp edge formation on the overlying portions of the return electrode 2501.2. Reducing sharp edges on the return electrode may mitigate unwanted electronic contact sequelae such as burns. In some embodiments, CiCTD tip return electrode 2501.2 may have any geometric shape including a wire, and may be positioned along any side or portion of the modular CiCTD tip 2501 and/or shaft 2502 excluding the recessions. It may be possible that sharp edges and/or corners and/or surface irregularities and/or surface defects on return electrode 2501.2 may contribute to a concentration of energy transfer when touched to a portion of the patient. Therefore, in some embodiments, it may be beneficial to have rounded and/or radiused edges and/or rounded corners. Surgeons may have a higher likelihood of an unwanted inadvertent ‘touch’ of adjacent patient tissue from the smaller of the sides; therefore in some embodiments, return electrode 2501.2 may be located on one or more of the larger substantially planar aspects of CiCTD tip 2501. In the depicted embodiment, an electrical insulator 2501.1 covers the edge of the return electrode closest to the active electrodes. Electrical insulator 2501.1 may comprise a band positioned along the periphery of return electrode 2501.2. In the depicted embodiments, the electrical insulator comprises polytetrafluoroethylene. In other contemplated embodiments, the electrical insulator may comprise an electrically nonconductive polymer with a high melting temperature. In some embodiments, the nonconductive polymer may comprise for example, polyether etherketone and/or polysulfone, etc. In other contemplated embodiments, the electrical insulator may comprise an electrically nonconductive and/or thermally nonconductive anti-stick polymer. In some embodiments a nonconductive coating may cover any portion of a return electrode and/or may have any shape. In some embodiments, the electrical insulator may cover about 20% of the return electrode surface. In other embodiments, the electrical insulator may cover a percentage of the return electrode that may range from 1% to 90%. In other embodiments, a portion and/or all of a return electrode may be covered with a porous substance of a lower relative static permittivity such that electrical charge transfer is reduced, such as alumina. Such a porous coverage may reduce the chance of a tissue burn from inadvertent touching and/or heating.

In some embodiments, electrodes may be printed onto a ceramic and/or nonconductive surface by various methods, for example using the Micropen™ of OhmCraft technique. In some embodiments, return electrodes 2501.2 may be printed onto a ceramic and/or nonconductive surface. In some embodiments, lysing elements comprising electrodes may be printed on or about the relative recessions.

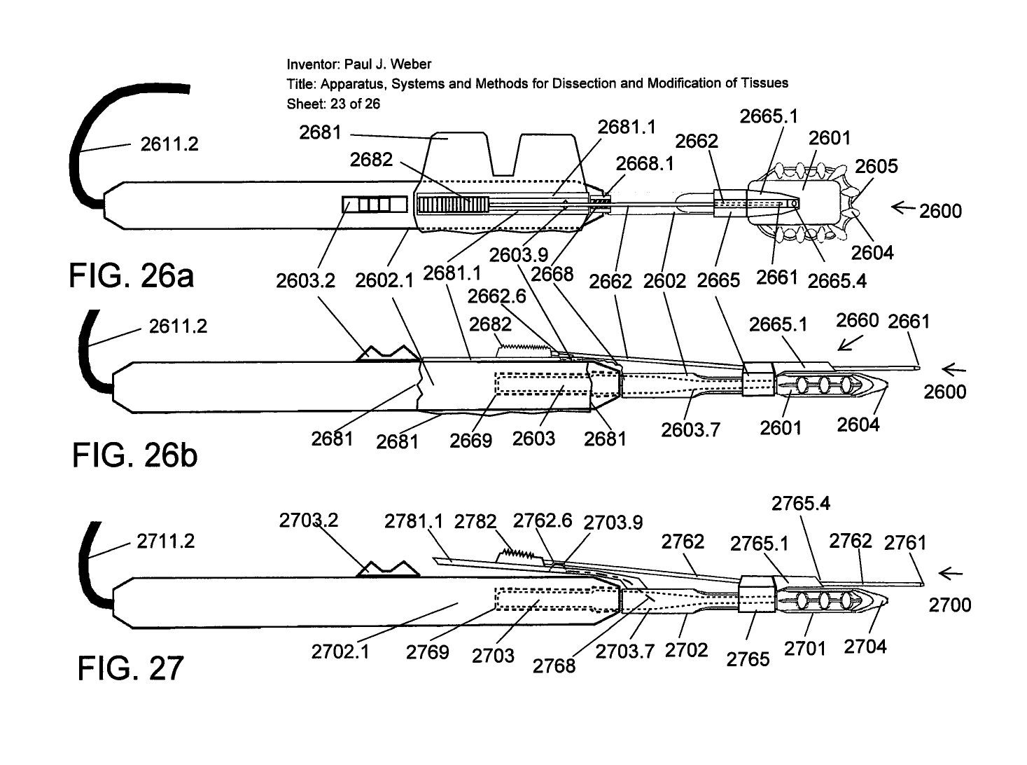

FIGS. 26a,b depict an embodiment comprising an axially advanceable SC 2600. SC comprises SC shaft 2662, SC tip 2661 and/or SC coupler 2665. In the depicted embodiment, the Modular TD comprises a Modular TD tip 2601, a Modular TD shaft 2602, a Modular TD plug 2603. The modular TD 2600 depicted in this embodiment has non-axial protrusions. In other embodiments, the modular TD may comprise axial protrusions and lack non-axial protrusions.

SC shaft may be coupled with modular TD by way of coupler 2665. The depicted embodiment also comprises axial protrusions, such as 2604, and axial lysing segments 2605 as well as non-axial & transitional protrusions. Transitional protrusions, axial protrusions, non-axial protrusions, axial lysing elements, non-axial lysing elements and lysing elements of differing geometries are discussed in more detail elsewhere in this application. Modular TD 2600 is modular in that it is removable from an electrosurgical ‘pencil’ shaft 2602.1 and/or an electrosurgical ‘pencil’ handle. Rocker switch 2603.2 may control the electrosurgical energy such as a suitable waveform derived from an electrosurgical generator (not seen in this view) brought into the handle via conduit 2611.2. In the depicted embodiment modular TDM tip plug 2603 is configured to be received within a corresponding electrosurgical ‘pencil’ shaft recess 2669 formed within electrosurgical ‘pencil’ shaft 2602.1. In some embodiments, elements within modular TDM tip plug 2603 and/or electrosurgical ‘pencil’ shaft recess 2669 may be electrically connected with electrical elements within ‘pencil’ shaft 2602.1 and/or ‘pencil’ handle which are in turn connected with ‘pencil’ switch 2603.2 and/or conduit 2611.2.

So as not to have the surgeon set down the TD or TDM, to pick up other instrumentation from the surgical tray to stem bleeding blood vessel(s), it may be beneficial during some surgical procedures to have spot coagulator coagulation capabilities within the same instrument.

In the depicted embodiment, modular TD shaft 2602 may comprise an electrical insulator and/or thermally insulating and/or supportive coating and/or cover overlying a conductive material and/or metal axial core (shown in phantom) 2603.7; In some embodiments, the conductive material may comprise: steel, nickel, alloys, palladium, gold, tungsten, silver, copper, platinum and/or any other conductive metal that does not give off toxic residua at operating temperatures. In other contemplated embodiments, the conductive material may comprise cermets and the like. In some embodiments, the electrical insulator may comprise, for example, porcelain, ceramics, glass-ceramics, plastics, various halogenated carbon molecules, polytetrafluoroethylene, carbon, graphite, and graphite-fiberglass composites and the like. In alternative embodiments the electrical insulator may comprise polyether etherketone and/or polysulfone and/or another electrically nonconductive polymers (with thermal stability in the operating range) and/or materials that are both electrically non-conductive and of low thermal conductivity. SC tip 2661 shapes may comprise those discussed in other tip embodiments in this application. In the depicted embodiment SC shaft 2662 is slidably coupled to the modular TD shaft 2602 of the modular TD by SC coupler 2665. SC coupler 2665 is one example of a means for moveably coupling a SC with a surgical tool. SC coupler 2665 may be configured to allow SC shaft 2662 to slide relative to SC coupler 2665 and modular TD shaft 2602. In the depicted embodiment, SC coupler 2665 is rigidly affixed to TD shaft 2662. In contemplated embodiments, SC coupler 2665 may be configured to slide relative to TD shaft 2602. In such embodiments, SC coupler 2665 may be rigidly affixed to SC shaft 2662. In some embodiments, SC coupler 2665 may comprise a sterilizable plastic and/or polymer and/or ceramic and/or polytetrafluoroethylene and/or other non-conductive and/or insulating material. In some embodiments, SC coupler 2665 may comprise an integral part of modular TD shaft 2602. In contemplated embodiments, SC coupler 2665 may comprise an integral part of modular TD tip 2601. In other embodiments SC coupler 2665 may comprise a separate component that may be coupled with modular TD tip 2601 by way of for example form fitting, snap fitting, an adhesive, welding, and the like. One or more passageways in SC coupler 2665 and/or SC sheath 2665.1 may allow the SC shaft 2662 to pass therethrough. In some contemplated embodiments, SC coupler 2665 may be formed from and/or be an integral part of modular TD shaft 2602; in some of these embodiments, SC coupler 2665 may comprise the same insulating materials of modular TD shaft 2602. In other contemplated embodiments, SC coupler 2665 may be formed from and/or be an integral part of SC shaft 2662; in some of these embodiments, SC coupler 2665 may comprise the same insulating materials of SC shaft 2662. Some contemplated embodiments may lack SC sheath 2665.1.

Coupler 2665 may be configured to allow SC shaft 2662 to be advanced or retracted axially therethrough. The SC depicted in these figures may be coupled with electrosurgical ‘pencil’ shaft 2602.1 by way of modular TD plug 2603.

Means for selectively moving a SC (Spot Coagulator Moving Means=SCMM) 2681.1 (an example of such SCMM may include rails, grooves, tracks, ratchets, cables, arms, lines, etc.) may be affixed to ‘pencil’ shaft 2602.1. SCMM 2681.1 may be affixed to ‘pencil’ shaft 2602.1 by means for rigidly affixing SCMM (MFRA-SCMM) 2681; an example of such a means may include adhesive backed tapes, sheaths, films, adhesive backed foam pads, adhesive backed pads, hook & loop fastening systems, and the like. In the depicted embodiment, SC slidable toggle 2682 is affixed to SC shaft 2662 and moves with the distal (opposite of tip) end of the SC shaft between two rails 2681.1. Moving SC slidable toggle 2682 toward the modular TD tip 2601 within the SCMM may allow SC tip 2661 to be advanced axially to an operational position and retracted axially to a storage position. SC tip 2661 may retract into SC sheath 2665.1 via SC opening 2665.4 when SC slidable toggle 2682 is moved rearward. Passage of SC shaft through SC opening 2665.4 may be facilitated by such insulations as polytetrafluoroethylene. In some embodiments, the edges of SC opening 2665.4 may be serrated and/or irregularly edged and/or possess bristles and/or other texture that may facilitate dislodgement of electrocoagulation char and/or carbon to clean SC tip 2661. SC slidable toggle 2682 may comprise a grip, which may in some embodiments comprise a plurality of ridges, to facilitate advancement and retraction of SC shaft 2662. Some embodiments may further comprise a SC sheath 2665.1 wherein at least a portion of SC may be positioned in the storage position. In some embodiments SC sheath may extend from modular TD tip 2601. In other embodiments SC sheath 2665.1 may extend from SC coupler 2665. SC sheath may comprise an insulating material and may be configured to completely cover SC tip 2661 in the storage position. SC sheath 2665.1 may, in some embodiments, comprise a transparent material to allow a surgeon to visualize the position of SC tip 2661 within the sheath. Some embodiments may be further configured to clean SC tip 2661 upon withdrawal into SC sheath 2665.1 and/or upon protrusion through SC sheath 2665.1. In some contemplated embodiments SC coupler 2665 and/or SC sheath 2665.1 may be absent. MFRA-SCMM 2681 may be configured to accommodate ‘pencil’ shafts of varying cross sectional geometries and/or diameters and/or lengths. In the depicted embodiment, SC slidable toggle 2682 is attached to the screw-threaded proximal end of conductive metal core of SC shaft 2662; insulation may be removed in the threaded area of the SC shaft to improve the fit. In other contemplated embodiments, MFRA-SCMM 2681 may be attached to SC shaft 2662 by way of for example form fitting, snap fitting, an adhesive, & the like.

SC 2600 is electrically coupled with modular TD by SC electrical contact 2603.9 which may comprise conductive metal button. In contemplated embodiments SC electrical contact may comprise a conductive spring and/or a terminus of a wire and/or terminus of a conductive strip and the like. SC electrical contact 2603.9 may be coupled to SC electrical conduit 2668 which may be permanently affixed or releasably affixed to modular TD plug 2603 and/or shaft 2602. SC electrical conduit 2668 may be configured to accommodate pulling forces and/or movement of SC as it is placed on the electrosurgical pencil. For example, the depicted embodiment includes protective insulator 2668.1 which may be a plastic or other nonconductive film and/or coating that may fully wrap around the conductor & prevent the conductive strip within from being torn, for example, while the modular TDM and/or SC is being applied into the electrosurgical pencil.

An implementation using the depicted embodiment may involve the surgeon pushing distally SC slidable toggle 2682 which forces SC shaft 2662 distally through SC coupler 1265. At a distal point on the movement of SC slidable toggle 2682, whereupon SC shaft conductive opening 2662.6 is brought into direct contact with SC electrical contact 2603.9 which itself is in direct or indirect contact with the SC electrical conduit 2668 which itself is in direct or indirect contact with the conductive portion of modular TD shaft 2603.7. Thus electrosurgical energy such as a suitable waveform will be delivered, when the electrosurgical generator is activated, via modular TD plug 2603, into modular TD shaft 2602, into SC shaft 2662 and thereupon to SC tip 2661 and then into target tissue. In the depicted embodiment, the SC tip 2661 extends from the SC shaft 2662 and is conductive and not insulated along at least a portion of the tip. In some embodiments, the entire SC tip 2661 is conductive. In the depicted embodiment SC shaft 2662 may comprise stainless steel and may be round in cross-section. In the depicted embodiment, SC shaft 2662 may comprise an electrically insulating and/or supportive coating and/or cover overlying a conductive material and/or metal axial core 2603.7. In some embodiments, the conductive material may comprise: steel, nickel, alloys, palladium, gold, tungsten, silver, copper, platinum and/or any other conductive metal that does not give off toxic residua at operating temperatures. In other contemplated embodiments, the conductive material may comprise cermets and the like. In some embodiments, the electrical insulator may comprise, for example, porcelain, ceramics, glass-ceramics, plastics, various halogenated carbon molecules, polytetrafluoroethylene, carbon, graphite, and graphite-fiberglass composites and the like. In alternative embodiments the electrical insulator may comprise polyether etherketone and/or polysulfone and/or another electrically nonconductive polymers (with thermal stability in the operating range) and/or materials that are both electrically non-conductive and of low thermal conductivity. In the depicted embodiment, SC tip 2661 is shaped like the frustum of a cone. In some embodiments, SC shaft 2662 may be oval, flat, rectangular or geometric in cross-section or substantially flattened. In alternative embodiments, SC tip 2661 may be pointed, bullet shaped, or geometric in cross section; more angulate and/or pointed tips may disperse electrical energy more readily and allow greater precision than larger, more rounded tip designs. In the depicted embodiments of the SC shaft 2662, the electrical insulator may comprise polytetrafluoroethylene.

Although the depicted embodiment shows a manually deployed SC, other contemplated embodiments may allow deployment to be (including but not limited to): motorized and/or spring activated and/or screw driven and/or ratchet style and/or cog style and/or pneumatic and/or hydraulic, etc. Although, the embodiment depicted in FIG. 26a shows the SC shaft 2662 pointing about axially (relative to the modular TD axis) and/or passing about over the middle of the front of the modular TD tip 2601, in other contemplated embodiments, SC shaft 2662 may point at an angle within about 30 degrees of the axis of the modular TD and/or may pass over any other points on the modular TD tip 2601 besides the middle front of the modular TD tip. In other contemplated embodiments, SC shaft 2662 may point at angles greater than 30 degrees of the axis of the modular TD. In some embodiments, SC shaft 2662 may be flexible and its forward axis redirected upon passing through SC coupler 2665; in some embodiments SC coupler may pivot and/or rotate and/or have one or more non-axial passageways for SC shaft 2662 that may redirect the axis and/or pointing of the forward part of SC shaft 1262. For example, some embodiments may comprise a plurality of passageways for SC shaft 2662 each pointing in a different direction. In such embodiments one or more such passageways may be axial and one or more may be non-axial. In other contemplated embodiments, SC coupler 2665 and/or SC sheath 2665.1 may have one or more pathways that may lead to SC shaft exiting at a similar range of angles from the axis of the modular TD.

In other contemplated embodiments, a variety of geometrically shaped lysing elements and/or protrusions may co-exist side by side. For example, in some embodiments, two adjacent protrusions may differ in shape and/or size, 2 adjacent lysing elements may differ in length and/or shape and/or TD tip 2601 may otherwise be nonsymmetrical in one or more views. In the depicted embodiment, the SC tip 2661 and SC shaft 2662 together may measure about, 55 mm in length and about 3 mm in maximal thickness; the SCMM rails may measure 5 mm wide & 30 mm long; MFRA-SCMM may measure 30×20 mm; the SC toggle 2682 may be capable of moving about 80% of the rail length; the overall SC 2660 may measure about 75 mm in length. In embodiments wherein an electrical insulation and/or polymeric insulating coating may be present on such parts, for example on SC shaft 2662 such insulation may measure about 0.5 mm in thickness. In some embodiments, the insulation thickness may range from about 0.1 mm to 3 mm. Embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses.

FIG. 27 depicts an embodiment in which the SC 2700 may be an integral part of the modular TD. In the depicted embodiment, SC 2700 comprises: Spot Coagulator Moving Means (SCMM) 2781.1 which is rigidly affixed to TD shaft 2702, SC shaft 2762, SC tip 2761 and/or SC coupler 2765. SC moving means 2781.1 may further comprise electric lead 2768 which may be further electrically coupled to modular TD shaft conductive axial core 2703.7. SCMM 2781.1 may be configured to encase electric lead 2768 which may extend to SC electrical contact 2703.9. Electrical contact 2703.9 may be configured to electrically couple with a corresponding electrical contact 2762.6 or conductive element, which may comprise a bare portion of an insulator of an electrical contact. Electrical contact 2762.6 may be positioned on or within SC shaft 2762.

Some embodiments may be configured to extend in a shape that at least substantially matches a distal end of an existing surgical tool such as Bovie electrosurgical ‘pencil’. Some embodiments may further be configured such that 2781.1 is malleable or flexible so as to allow a surgeon to conform the SC to the electrosurgical tool.

FIG. 27 depicts an embodiment that differs from the embodiment depicted in FIGS. 26a,b in that the embodiment of FIG. 27 comprises: a Spot Coagulator Moving Means (SCMM) 2781.1 which is rigidly affixed to TD shaft 2702. FIG. 27 depicts an embodiment that also differs from the embodiment depicted in FIGS. 26a,b in that the embodiment of FIG. 27 lacks: means for rigidly affixing SCMM 2681 to modular TD shaft 2702.1 or modular TD handle. In some contemplated embodiments, an adhesive pad placed below SCMM may affix the SCMM to the modular TD handle with minimal rigidity. In other contemplated embodiments, the adhesive pad placed between the SCMM & modular TD handle affixes the two with strong rigidity.

Each of the other elements depicted in FIG. 27 may be identical to the corresponding elements shown in FIGS. 26a,b and are referenced by like numerals (numbers higher by 100). For example, FIG. 27 depicts an embodiment comprising an axially advanceable SC 2700. SC further comprises SC shaft 2762, SC tip 2761 and/or SC coupler 2765. In the depicted embodiment, the Modular TD comprises a Modular TD tip 2701, a Modular TD shaft 2702, a Modular TD plug 2703. The modular TD 2700 depicted in this embodiment has non-axial protrusions & axial protrusions 2704. The SC shaft may be coupled with modular TD by way of SC coupler 2765. Modular TD 2700 is modular in that it is removable from an electrosurgical ‘pencil’ shaft 2702.1 and/or an electrosurgical ‘pencil’ handle. Rocker switch 2703.2 may control the electrosurgical energy such as a suitable waveform derived from an electrosurgical generator (not seen in this view) brought into the handle via conduit 2711.2. In the depicted embodiment, modular TDM tip plug 2703 is configured to be received within a corresponding electrosurgical ‘pencil’ shaft recess 2769 formed within electrosurgical ‘pencil’ shaft 2702.1.

So as not to have the surgeon set down the TD or TDM, to pick up other instrumentation from the surgical tray to stem bleeding blood vessel(s), it may be beneficial during some surgical procedures to have spot coagulator coagulation capabilities within the same instrument.

In the depicted embodiment SC shaft 2762 is slidably coupled to the modular TD shaft 2702 by SC coupler 2765. SC coupler 2765 is one example of a means for moveably coupling a SC with a surgical tool. SC coupler 2765 may be configured to allow SC shaft 2762 to slide relative to SC coupler 2765 and modular TD shaft 2702.

In the depicted embodiment, means for selectively moving a SC (Spot Coagulator Moving Means=SCMM) 2781.1 (an example of such SCMM may include rails, grooves, tracks, ratchets, cables, arms, lines, etc.) is affixed to modular TD shaft 2702. In the depicted embodiment, SC slidable toggle 2782 is affixed to SC shaft 2762 and moves with the distal (opposite of tip) end of the SC shaft between two rails 2781.1. Moving SC slidable toggle 2782 toward the modular TD tip 2701 within the SCMM may allow SC tip 2761 to be advanced axially to an operational position and retracted axially to a storage position. SC tip 2761 may retract into SC sheath 2765.1 via SC opening 2765.4 when SC slidable toggle 2782 is moved rearward. In some contemplated embodiments SC coupler 2765 and/or SC sheath 2765.1 may be absent.

SC 2700 is electrically coupled with modular TD by SC electrical contact 2703.9 which may comprise conductive metal button. In contemplated embodiments SC electrical contact may comprise a conductive spring and/or a terminus of a wire and/or terminus of a conductive strip and the like. SC electrical contact 2703.9 may be coupled to SC electrical conduit 2768 which may be permanently encased inside a SCMM 2781.1 and may be affixed or releasably affixed to modular TD plug 2703 and/or shaft 2702. In the depicted embodiment, SCMM 2781.1 comprises a polypropylene. In other embodiments, SCMM may comprise a nonconductive polymer and/or other nonconductive plastic and/or nonconductive material and/or malleable metallic pieces (which may provide ‘flex’ & ‘memory’ to the SCMM. In some embodiments, the SCMM may be styled to some degree look a bit like a fencing foil guard. In the depicted embodiment, SC shaft 2762 may comprise an electrically insulating and/or supportive coating and/or cover overlying a conductive material and/or metal axial core (shown in phantom) 2703.7.

Although the depicted embodiment shows a manually deployed SC, other contemplated embodiments may allow deployment to be (including but not limited to): motorized and/or spring activated and/or screw driven and/or ratchet style and/or cog style and/or pneumatic and/or hydraulic, etc. Although, the embodiment depicted in FIG. 27 shows the SC shaft 2762 pointing about axially (relative to the modular TD axis) and/or passing about over the middle of the front of the modular TD tip 2701, in other contemplated embodiments, SC shaft 2662 may point at an angle within about 30 degrees of the axis of the modular TD and/or may pass over any other points on the modular TD tip 2701 besides the middle front of the modular TD tip. In other contemplated embodiments, SC shaft 2762 may point at angles greater than 30 degrees of the axis of the modular TD. In some embodiments, SC shaft 2762 may be flexible and its forward axis redirected upon passing through SC coupler 2765; in some embodiments SC coupler may pivot and/or rotate and/or have one or more non-axial passageways for SC shaft 2762 that may redirect the axis and/or pointing of the forward part of SC shaft 2762. For example, some embodiments may comprise a plurality of passageways for SC shaft 2762 each pointing in a different direction. In such embodiments one or more such passageways may be axial and one or more may be non-axial. In other contemplated embodiments, SC coupler 2765 and/or SC sheath 2765.1 may have one or more pathways that may lead to SC shaft exiting at a similar range of angles from the axis of the modular TD.

In other contemplated embodiments, a variety of geometrically shaped lysing elements and/or protrusions may co-exist side by side. For example, in some embodiments, two adjacent protrusions may differ in shape and/or size, 2 adjacent lysing elements may differ in length and/or shape and/or TD tip 2701 may otherwise be nonsymmetrical in one or more views.

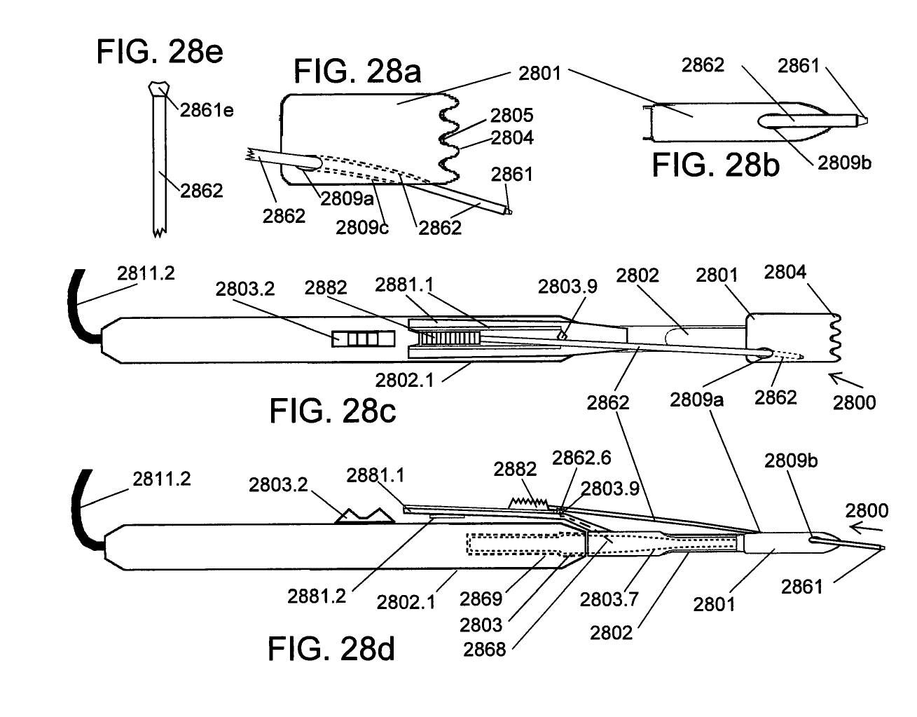

In the depicted embodiment of FIG. 27, measurements about those of similar parts in FIGS. 26a,b embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses. As is similar to FIG. 26, SCMM rails may measure 5 mm wide & 30 mm long, however, in the embodiment depicted in FIG. 27, in which the SCMM 2781.1 will be similar to that shown in FIG. 28, the SCMM may be part of a polymeric protrusion extending from the modular TD shaft that passes over the shaft of the electrosurgical ‘pencil’ 2702.1. In some embodiments, the SCMM may be shaped slightly resembling the curviform guard of a fencing foil with a width of about 1 cm and a length of about 45 mm. In some contemplated embodiments an adhesive placed on the bottom of the SCMM and/or between the SCMM and the shaft of the electrosurgical ‘pencil’ may affix the two parts to some degree.

FIG. 28a,b,c,d depict an embodiment in which the SC 2800 may be an integral part of the modular TD. FIGS. 28a,b,c,d depict an embodiment that differs from the embodiment depicted in FIGS. 26a,b in that the embodiment of FIGS. 28a,b,c,d comprises: two modular TD tip openings 2809a & 2809b, modular TD tip passageway 2809c & Spot Coagulator Moving Means (SCMM) 2881.1 which is rigidly affixed to TD shaft 2802. SC moving means 2881.1 may further comprise electric lead 2868 which may be further electrically coupled to modular TD shaft conductive axial core 2803.7. SCMM 2881.1 is configured to encase electric lead 2868 which may extend to SC electrical contact 2803.9. FIG. 28 depicts an embodiment that also differs from the embodiment depicted in FIGS. 26a,b in that the embodiment of FIG. 28 lacks: SC coupler 2665, SC sheath 2665.1, & means for rigidly affixing SCMM 2681 to modular TD shaft 2702.1 or modular TD handle. However, in the embodiment depicted in FIG. 28d, an adhesive pad 2881.2 is positioned below SCMM, which may be used to affix the SCMM 2881.1 to the electrosurgical ‘pencil’ shaft and/or handle.

Each of the other elements depicted in FIG. 28 may be identical to the corresponding elements shown in FIGS. 26a,b and are referenced by like numerals (numbers higher by 200). For example, in the depicted embodiment, SC 2800 comprises: Spot Coagulator Moving Means (SCMM) 2881.1 which is rigidly affixed to TD shaft 2802, SC shaft 2862, and/or SC tip 2861. SC moving means 2881.1 may further comprise electric lead 2868 which may be further electrically coupled to modular TD shaft conductive axial core 2803.7. SCMM 2881.1 may be configured to encase electric lead 2868 which may extend to SC electrical contact 2803.9. Some embodiments may be configured to extend in a shape that at least substantially matches a distal end of an existing surgical tool such as Bovie electrosurgical ‘pencil’. Some embodiments may further be configured such that 2881.1 may be malleable and/or flexible so as to allow a surgeon to conform the SC to the electrosurgical tool.

In the depicted embodiment, the Modular TD comprises a Modular TD tip 2801, a Modular TD shaft 2802, a Modular TD plug 2803. The modular TD 2800 depicted in this embodiment comprises axial protrusions 2804 and lysing elements 2805. In other embodiments, the modular TD may comprise axial, non-axial and/or transitional protrusions as discussed elsewhere in this application. Modular TD 2800 is modular in that it is removable from an electrosurgical ‘pencil’ shaft 2802.1 and/or an electrosurgical ‘pencil’ handle. Rocker switch 2803.2 may control the electrosurgical energy such as a suitable waveform derived from an electrosurgical generator (not seen in this view) brought into the handle via conduit 2811.2. In the depicted embodiment, modular TDM tip plug 2803 is configured to be received within a corresponding electrosurgical ‘pencil’ shaft recess 2869 formed within electrosurgical ‘pencil’ shaft 2802.1.

So as not to have the surgeon set down the TD or TDM, to pick up other instrumentation from the surgical tray to stem bleeding blood vessel(s), it may be beneficial during some surgical procedures to have spot coagulator coagulation capabilities within the same instrument.

In the depicted embodiment SC shaft 2862 is slidably coupled to the modular TD tip 2801 by two modular TD tip openings 2809a & 2809b & modular TD tip passageway 2809c. Modular TD tip openings 2809a & 2809b & modular TD tip passageway 2809c may be configured to allow SC shaft 2862 to slide relative to modular TD tip 2801.

In the depicted embodiment, Means for selectively moving a SC (Spot Coagulator Moving Means=SCMM) 2881.1 (an example of such SCMM may include rails, grooves, tracks, ratchets, cables, arms, lines, etc.) is affixed to modular TD shaft 2802. In the depicted embodiment, SC slidable toggle 2882 is affixed to SC shaft 2862 and moves with the distal (opposite of tip) end of the SC shaft between two rails 2881.1. Moving SC slidable toggle 2882 toward the modular TD tip 2801 within the SCMM may allow SC tip 2861 to be advanced axially to an operational position and retracted axially to a storage position. SC tip 2861 may retract into modular TD tip 2801 via modular TD tip opening 2809b when SC slidable toggle 2882 is moved rearward.

SC 2800 is electrically coupled with modular TD by SC electrical contact 2803.9 which may comprise conductive metal button. In contemplated embodiments SC electrical contact may comprise a conductive spring and/or a terminus of a wire and/or terminus of a conductive strip and the like. SC electrical contact 2803.9 may be coupled to SC electrical conduit 2868 which may be permanently encased inside a SCMM 2881.1 and/or may be affixed or releasably affixed to modular TD plug 2803 and/or shaft 2802. In the depicted embodiment, SCMM 2881.1 comprises a polypropylene. In other embodiments, SCMM may comprise a nonconductive polymer and/or other nonconductive plastic and/or nonconductive material and/or malleable metallic pieces (which may provide ‘flex’ & ‘memory’ to the SCMM. In some embodiments, the SCMM may be styled to some degree look a bit like a fencing foil guard. In the depicted embodiment, SC shaft 2862 may comprise an electrically insulating and/or supportive coating and/or cover overlying a conductive material and/or metal axial core (shown in phantom) 2803.7.

Although the depicted embodiment shows a manually deployed SC, other contemplated embodiments may allow deployment to be (including but not limited to): motorized and/or spring activated and/or screw driven and/or ratchet style and/or cog style and/or pneumatic and/or hydraulic, etc. Although, the embodiment depicted in FIG. 28 shows the SC shaft 2862 pointing in a non-axial angle (relative to the modular TD axis), in other contemplated embodiments, SC shaft 2862 may point at an angle within about 45 degrees of the long axis of the modular TD shaft. In other contemplated embodiments, SC shaft 2862 may point at angles greater than 45 degrees of the long axis of the modular TD. In some embodiments, SC shaft 2862 may be flexible and may be able to be redirected upon passing through TD tip 2801. In some embodiments, modular TD tip may comprise one or more passageways that may SC shaft 2862 that may redirect the axis and/or pointing of the forward part of SC shaft 2862. For example, some embodiments may comprise a plurality of passageways for SC shaft 2862 each pointing in a different direction. In such embodiments one or more such passageways may be axial and one or more may be non-axial.

Modular TD tip openings 2809a & 2809b, are formed on modular TD tip 2801 such that passageway 2809c extends therebetween.

The said passageway 2809c may be watertight to prevent ionic fluids from reaching other electrical components within the TD tip. In the depicted embodiment, SC tip 2861 may only able to conduct electrosurgical energy delivered by the ‘pencil’ when the toggle 2882 has engaged the aforementioned contacts 2862.6 & 2803.9. In further contemplated embodiments, SC tip 2861 may conduct electrosurgical energy when toggle or SC tip is a range of positions including but not limited to ‘not deployed.’ In contemplated embodiments, electrical contacts may comprise any known in the art.

In contemplated embodiments, SC shaft 2862 may emanate axially from modular TD tip 2801. In some embodiments, a thicker modular TD tip may comprise modular TD tip openings & an axially directed modular TD tip passageway emanating above and/or below lysing elements and/or protrusions. In other contemplated embodiments, a thicker modular TD tip may comprise modular TD tip openings & a non-axially directed modular TD tip passageway. In other contemplated embodiments, SC tip 2861 may emanate within 15 degrees of axially from modular TD tip 2801.

In contemplated embodiments, SC shaft 2862 may emanate axially from modular TD tip 2801 wherein frontal modular TD tip opening 2809b, &/or passageway 2809c may emanate from an axial relative recession & thus adjacent one or more axial protrusions. The SC tip may be positioned at and/or about the orifice of TD tip opening 2809b so that SC tip 2861 may apply the selected electrosurgical waveform for cutting and/or ‘coag’ and/or blend settings (to perform as if it were a shaped lysing segment, for example concave, convex, beveled and/or straight as described elsewhere in this application) and/or if deployed (extended) for spot coagulation, SC tip 2861 may perform spot coagulation. In another contemplated embodiment, as depicted in FIG. 28e, the forward (distal) aspects of an SC tip 2861e (mounted on a SC shaft 2862) may be modified and/or fashioned to resemble and/or function as a lysing element. Thus, when such a lysing-element-like-SC tip 2861e may be positioned at and/or about the orifice of TD tip opening 2809b (wherein frontal modular TD tip opening 2809b, &/or passageway 2809c may emanate from the base/proximal location of a relative recession) the lysing-element-like-SC tip 2861e may benefit from physical characteristics it may share with various lysing element shapes described in this application. Lysing element design has been described in detail elsewhere in this application. In another contemplated embodiment, as depict in FIG. 28e, the lysing-element-like-SC tip 2861e may in a storage position comprise one of the lysing elements positioned within a recession of TD tip 2801. In such embodiments, lysing-element-like-SC tip 2861e may be configured to extend out of its corresponding recession as SC 2862 extends forward to a Spot Coagulating operational position. When the lysing-element-like-SC tip 2861e is in a storage position (resting between protrusions in a relative recession similar to its counterpart lysing element 2805) the lysing-element-like-SC tip 2861e may be ‘operational’ for hemostatic tissue dissection purposes as opposed to spot coagulation purposes.

In other contemplated embodiments, a variety of geometrically shaped lysing elements and/or protrusions may co-exist side by side. For example, in some embodiments, two adjacent protrusions may differ in shape and/or size, 2 adjacent lysing elements may differ in length and/or shape and/or TD tip 2801 may otherwise be nonsymmetrical in one or more views.

In the depicted embodiment of FIG. 28, measurements about those of similar parts in FIGS. 28a,b, embodiments are contemplated wherein sizes of about one-fifth to about five times these dimensions may have possible uses. It is also contemplated in some veterinary embodiments; tip sizes of about one-tenth to 20 times the aforementioned dimensions may also have possible uses. As is similar to FIGS. 26 &/or 27, SCMM rails may measure 5 mm wide & 30 mm long, however, in the embodiment depicted in FIG. 28, in which the SCMM 2881.1 will be similar to that shown in FIG. 27, the SCMM may be part of a polymeric protrusion extending from the modular TD shaft that passes over the shaft of the electrosurgical ‘pencil’ 2802.1. In some embodiments, the SCMM may be shaped slightly resembling the curviform guard of a fencing foil with a width of about 1 cm and a length of about 45 mm. In some contemplated embodiments an adhesive placed on the bottom of the SCMM and/or between the SCMM and the shaft of the electrosurgical ‘pencil’ may affix the two parts to some degree.

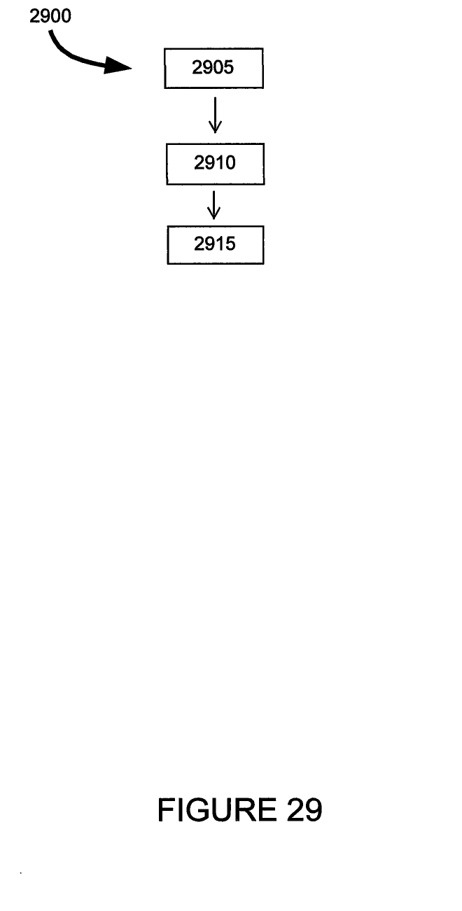

FIG. 29 (was 24) illustrates an implementation of a method 2900 according to this disclosure for making an electrosurgical incision using the lysing elements of a TD in tissue and/or tissue plane with the assistance of a TD. In some implementations, surgeon(s) may need to incise layers of tissues in order to access tissue and/or an organ to repair or treat it. In some implementations, the skin surrounding the anticipated entrance wound for the surgical area may be cleansed by, for example, with isopropyl alcohol (degreaser) followed by germicidal chlorhexidine scrub. Then, a local anesthetic may be applied (such as by injecting) 1% lidocaine+1:100,000adrenaline to the skin.

Step 2905 may comprise the initial skin opening or body cavity opening steps of such a procedure. Step 2905 may comprise forming an incision. In some implementations, step 2905 may comprise making the incision using the same surgical tool used to perform every other step in method 2900. In other implementations step 2905 may be performed in part with the use of another surgical instrument such as the scalpel. For example, step 2905 may be initially performed with, for example, a #15 Bard-Parker™ Scalpel to a length of about 1 cm into the target tissue to be incised for example the epidermis and dermis. In some implementations, step 2905 may comprise making the initial skin incision using a standard a sharp surgical instrument such as for example, a scalpel and/or sharp scissors. Step 2910 may further comprise placing the tip of the TD tip in an orientation substantially perpendicular to the tissue plane to be cut with at least one protrusion lying on opposite sides of the plane of tissue to be incised and extending the initial incision using the TD tip. In some implementations, step 2910 may comprise using the TD tip in an unzipping fashion to completely separate the planar tissue into two separate surfaces. Step 2910 may further comprise energizing the lysing elements with electrosurgical energy. Step 2910 may further comprise energizing the lysing elements with electrosurgical cutting and/or coagulation and/or ‘blended’ waveforms. Step 2910 may further comprise pushing and/or moving forward the TD and its attendant lysing elements so as to cut the intended tissue layer and/or plane. Step 2915 may comprise positioning the tip of the TD in an orientation substantially perpendicular to a second tissue which may be adjacent to the first tissue plane referenced in step 2910 with at least one protrusion lying on opposite sides of the second tissue plane and using the lysing elements of the TD tip to cut the second tissue plane into separate surfaces. A variety of other steps may be performed before during or after the steps depicted in FIG. 29 as would be apparent to one of ordinary skill in the art. In some implementations, the tissues traversed may require closure by suturing and/or stapling and/or tissue glue and/or taping and/or binding. In some implementations, organs and/or organ systems that the TD may be useful to cut through include the surface skin and/or fascial layers and/or muscular layers and/or protecting layers (for example peritoneum) to access organs and organ systems that may include but need not be limited to muscle, and/or parotid, and/or salivary gland, and/or thyroid, and/or lung, and/or heart, and/or gastrointestinal, and/or liver, and/or pancreas, and/or spleen, and/or gallbladder, and/or kidney, and/or adrenal, and/or prostate, and/or ovary, and/or uterus, and/or bladder, and/or vascular, and/or lymph nodes and/or skeleton, and/or lung.

Any of the embodiments of TDM and/or TD discussed herein including, but not limited to, the embodiments discussed with FIGS. 1-30 etc. may be used in conjunction with one or more of the steps disclosed in connection with FIG. 29.

In a more general implementation of a method according to this disclosure for dissection and modification of tissues, a first step may comprise creating an incision into a patient's skin.

A second step may comprise inserting a Tissue Dissecting and Modifying Wand into the incision and positioning the Tissue Dissecting and Modifying Wand within the body. The Tissue Dissecting and Modifying Wand may comprise a tip having a plurality of protrusions with lysing elements positioned between the protrusions. The Tissue Dissecting and Modifying Wand may also comprise an energy window positioned on top of the Tissue Dissecting and Modifying Wand that is configured to deliver energy to modify tissues.

A third step may comprise fanning out the Tissue Dissecting and Modifying Wand to define a target region within which to dissect and modify tissues. This step may comprise separating tissue using the lysing element(s) to define the target region. During this step, in some implementations, the patient's target tissue may be placed under tension by stretching/tightening the skin at the target region during the fanning/tissue separation.

A fourth step may comprise activating the energy window and moving the energy window around within the target region for hemostasis and/or to induce postoperative fibrosis. Alternatively, the energy window may be activated prior to the third step such that the step of fanning out the Tissue Dissecting and Modifying Wand to define the target region also comprises heating tissues to induce fibrosis and/or hemostasis within the target region.

In another embodiment of a method for separating and modifying tissue using a tissue dissecting and modifying wand, the method may comprise creating an incision into a patient's skin. A tissue dissecting and modifying wand may be inserted into the incision. The tissue dissecting and modifying wand may comprise: a tip comprising a plurality of protrusions; at least one lysing element positioned between at least two adjacent protrusions among the plurality of protrusions; and an energy window configured to deliver energy to tissue adjacent to the tissue dissecting and modifying wand during a procedure. The energy window may comprise an electromagnetic emission energy window, and wherein the energy window is positioned and configured to deliver electromagnetic energy from the tissue dissecting and modifying wand to tissue adjacent to the tissue dissecting and modifying wand during a procedure.

The tissue dissecting and modifying wand may further comprise a ITOMSensor positioned on the tissue dissecting and modifying wand and configured to provide speed data (for example, the difference in speed of adjacent tissue versus a tissue dissecting and modifying wand tip during a procedure. In such implementations, data may be received from the tissue dissecting and modifying wand generated from the ITOMSensor, wherein the data allows a user to determine or control the energy deposited per unit of tissue surface within a patient's body during treatment using energy from an electromagnetic delivery energy window. In some implementations, the energy deposited per unit of tissue surface area may be derived from distance travelled by the TDM relative to the tissue surface. In other implementations, the speed data may be used by a related processor and/or accompanying software to automatically modify the energy delivered by the TDM.

In another implementation of a method for separating and modifying tissue using a tissue dissecting and modifying wand, the tissue dissecting and modifying wand may comprise a tip comprising a first plurality of protrusions and a second plurality of protrusions, wherein the first plurality of protrusions is positioned to at least substantially extend in a first direction, and wherein the second plurality of protrusions is positioned to at least substantially extend in a second direction distinct from the first direction; at least one lysing element positioned between at least two adjacent protrusions in the first plurality of protrusions; at least one lysing element positioned between at least two adjacent protrusions in the second plurality of protrusions; and a ITOMSensor positioned on the tissue dissecting and modifying wand and configured to provide speed of the tissue dissecting and modifying wand during a procedure. In such implementations, the method may comprise a step of receiving data from the tissue dissecting and modifying wand generated from the ITOMSensor, wherein the data allows a user to determine speed (for example, the difference in speed of adjacent tissue versus a tissue dissecting and modifying wand tip during a procedure). In this manner, the ITOMSensor may allow a user to determine &/or control the energy deposited per unit of tissue surface within a patient's body during treatment using energy from an electromagnetic delivery energy window. In other implementations, such as implementations involving use of a TD lacking an energy window, the ITOMSensor may allow the user to determine and/or control the rate of energy delivery from the lysing elements.

An example of an embodiment of an apparatus according to this disclosure for tissue dissection and modification may comprise:

a handle;

a tip comprising a plurality of protrusions having one or more lysing elements positioned between the protrusions; and

an energy window positioned on an upper side of the apparatus, wherein the energy window may comprise an electromagnetic emission energy window, and wherein the energy window is positioned and configured to deliver electromagnetic energy from the tissue dissecting and modifying wand to tissue adjacent to the tissue dissecting and modifying wand during a procedure.

In some embodiments, as described above, the electromagnetic emission energy window may comprise fiberoptics &/or mirrors configured to deliver LASER energy from the energy window.

In alternative embodiments, as described above, the electromagnetic emission energy window may comprise radiofrequency/electrosurgical energy delivering elements.

An example of an embodiment of an apparatus according to this disclosure for tissue dissection and modification may comprise:

a handle;

a tip comprising a plurality of protrusions having one or more lysing elements positioned between the protrusions; and

an electromagnetic emission energy window positioned on an upper side of the apparatus, wherein the electromagnetic emission energy window may comprise fiberoptics &/or mirrors configured to deliver LASER energy from the energy window.

In alternative embodiments, as described above, the electromagnetic emission energy window may comprise radiofrequency/electrosurgical energy delivering elements.

FIGS. 30a-c depict an embodiment of a surgical device comprising a TDM 3000. The TDM comprises a conductive element 3003.9 protruding from an electroconductive metallic shaft 3003. Conductive element 3003.9 is shaped like a fin, and may, as previously discussed, be configured to be selectively electrically coupled with another conductive element or elements in one or more operational positions. For example, in some embodiments, a spot coagulator may be movable relative to shaft 3003 between one or more storage positions and one or more operational positions. In the one or more operational positions, conductive element 3003.9 may be configured to contact a corresponding conductive element on the spot coagulator. Similarly, in the one or more storage positions, conductive element 3003.9 may be electrically isolated from the corresponding conductive element or elements such that the spot coagulator tip is not able to deliver coagulating electrical energy.

The TDM 3000 depicted in FIGS. 30a-30c further comprises a bovie electrosurgical pencil shaft 3002.1 and a spot coagulator comprising a spot coagulator shaft 3062 and a spot coagulator tip 3061. The spot coagulator is coupled to the TDM 3000 by way of SC coupler 3065.1. In the depicted embodiment, SC coupler 3065.1 comprises an integral extension of the surgical device tip 3001, which, as previously discussed, may comprise a plurality of protrusions 3004 and at least one lysing element 3005 positioned between each adjacent protrusion 3004. In some embodiments, SC coupler 3065.1 may also comprise a sheath configured to receive tip 3061 in a storage position.

Shaft 3003 may comprise a plug at a proximal end configured to be positioned in a distal end of a recess of a surgical tool, such as the Bovie pencil shaft 3002.1 depicted in the figures.

The embodiment depicted in FIGS. 30a-30c further comprises SC opening 3065.4, TD shaft conductive core 3003.7, and SC coupler conductive element 3003.9. TD shaft conductive core 3003.7 may form a lysing element plate 3005 at its distal end, which may be used to form a distal tip comprising the lysing elements of the TDM 3000 SC coupler conductive element 3003.9 comprises a protruding electrically-conductive element. More particularly, SC coupler conductive element 3003.9 comprises a corresponding contact that may be used to facilitate desired contact with a portion of the SC, such as an SC shaft projecting contact (hidden from view). SC tip 3061 may pass into and/or out of SC opening 3065.4.

Each of the other elements depicted in FIGS. 30a-c may be similar to corresponding elements shown in FIGS. 13a-b and are referenced by like numerals (numbers higher by 1700). For example, FIGS. 30a-c depict an embodiment comprising SC shaft 3062, SC tip 3061, SC toggle 3081, and/or SC sheath 3065.1. In some embodiments, TD 3000 may comprise a modular TD. The modular TD 3000 depicted in this embodiment comprises a modular TD tip 3001, modular TD shaft conductive core 3003.7, modular TD plug 3003 and also comprises axial protrusions 3004 and axial lysing elements 3005 but lacks non-axial protrusions and lysing segments. The standard electrosurgical ‘pencil’ depicted, comprises shaft 3002.1, and/or ‘pencil’ switch 3002.2. SC tip 3061 may retract into SC sheath 3065.1 via SC opening 3065.4. In some embodiments, SC opening 3065.4 may be formed within an SC gate. SC toggle 3081 may comprise a grip. Although the embodiment depicted in FIG. 30a shows the SC shaft 3062 pointing axially (relative to the modular TD axis) and/or passing about over the middle of the front of the modular TD tip 3001, in other contemplated embodiments, SC shaft 3062 may point at an angle within about 30 degrees of the axis of the modular TD and/or may pass over any other points on the modular TD tip 3001 besides the middle front of the modular TD tip. In other contemplated embodiments, SC shaft 3062 may point at angles greater than 30 degrees of the axis of the modular TD.

In the depicted embodiment, the SC connecting contact 3003.9 may comprise a conductive piece emanating and/or projecting from the conductive core of modular TD shaft 3003, for example a ‘fin’; such pieces may be manufactured by stamping and/or welding or other methods known in the art. An example of a protruding electrically-conductive element comprising a fin is shown in FIG. 30b. SC connecting contact 3003.9 may be configured to accommodate axial movement of SC shaft 3062 between one or more operational positions and one or more storage positions. For example, some embodiments may comprise a plurality of adjacent conductive projections and/or rings or other non-insulated areas such that SC tip 3061 may be positioned at a plurality of preset distances from modular TD tip 3001. In the depicted embodiment of FIG. 30a, SC connecting contact 3003.9 (hidden from view) is electrically coupled to the modular TD shaft 3062. In contemplated embodiments, SC connecting contact 3003.9 may be electrically coupled to the modular TD tip 3001 and/or modular TD plug 3003. In some embodiments, the electrically-conductive element may be flexible, such as spring-loaded or spring-biased, to further facilitate desired electrical contact.

In some embodiments, SC coupler 3065.1 may comprise at least one passageway to receive SC shaft 3062. In some embodiments, SC coupler 3065.1 may be an integral part of tip 3001, as shown in FIG. 30a and FIG. 30c. In embodiments comprising a shaft 3003 having a conductive core, the conductive core may comprise a lysing element plate 3005, and the lysing element(s) of tip 3001 may be part of the lysing element plate 3005, as depicted in FIGS. 30a-c.

In the depicted embodiment, the spot coagulator is configured to move axially with respect to an axis of the TDM 3000 between at least one operational position and at least one storage position. However, other embodiments are contemplated in which the spot coagulator movement need not be axial, either in whole or in part.

It will be understood by those having skill in the art that changes may be made to the details of the above-described embodiments without departing from the underlying principles presented herein. For example, any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

Furthermore, the described features, components, structures, steps, or characteristics may be combined in any suitable manner in one or more alternative embodiments and/or implementations. In other words, any of the features, components, structures, steps, or characteristics disclosed in any one disclosed embodiment may be combined with features, components, structures, steps, or characteristics of other disclosed embodiments.

Various examples of aspects of certain embodiments disclosed herein are described below in numbered clauses for convenience. These are provided as examples and do not limit the subject technology or scope of the inventions disclosed herein. It is noted that any of the dependent clauses may be combined in any combination, and placed into a respective independent clause.

### Example Embodiments

1. An apparatus for tissue separation and modification, comprising:
a tip comprising a plurality of protrusions;
at least one lysing element positioned between at least two adjacent protrusions in the plurality of protrusions; and
a spot coagulator comprising a spot coagulator tip configured to deliver energy for coagulating a blood vessel during a surgical procedure with the apparatus for tissue separation and modification.

2. The apparatus of claim 1, wherein the spot coagulator is configured to be positioned in a storage position and advanced relative to the tip of the apparatus to an operational position.

3. The apparatus of claim 2, further comprising a spot coagulator sheath, wherein at least a portion of the spot coagulator is configured to be positioned within the spot coagulator sheath in the storage position.

4. The apparatus of claim 3, wherein the sheath comprises a gate, wherein the spot coagulator tip passes through the gate as the spot coagulator is moved from the storage position to the operational position.

5. An apparatus for tissue separation, comprising:
a tip comprising a plurality of protrusions;
at least one lysing element positioned between at least two adjacent protrusions in the plurality of protrusions; and
a motion sensor configured to detect movement of the apparatus during a surgical procedure with the apparatus.

6. An apparatus for tissue separation and modification, comprising:
a tip comprising a plurality of protrusions;
at least one lysing element positioned between at least two adjacent protrusions in the plurality of protrusions; and
a spot coagulator comprising:
   a spot coagulator shaft; and
   a spot coagulator tip configured to deliver energy for coagulating a blood vessel during a surgical procedure with the apparatus for tissue separation and modification, wherein the spot coagulator tip is movable with respect to the plurality of protrusions.

7. The apparatus of claim 6, further comprising a shaft coupled to the tip, wherein the spot coagulator tip is axially movable with respect to the shaft.

8. The apparatus of claim 7, wherein the spot coagulator shaft is slidably coupled to the shaft.

9. The apparatus of claim 8, further comprising a spot coagulator coupler configured to receive the spot coagulator shaft and slidably couple the spot coagulator shaft to the shaft.

10. The apparatus of claim 9, wherein the spot coagulator coupler is an integral part of the shaft.

11. The apparatus of claim 7, further comprising a sheath, wherein the spot coagulator tip is configured to be received in the sheath in a storage position, and wherein the spot coagulator tip is configured to extend out of the sheath in an operational position.

12. The apparatus of claim 11, wherein the sheath is configured to clean the spot coagulator tip upon passing through the sheath.

13. The apparatus of claim 12, further comprising a gate coupled with the sheath, wherein the gate is configured to clean the spot coagulator tip upon passing through the gate.

14. The apparatus of claim 11, wherein the sheath comprises a plurality of passageways, and wherein the spot coagulator shaft is configured to be repositioned in any of the plurality of passageways.

15. The apparatus of claim 6, wherein the spot coagulator tip is configured to pivot about a base to selectively deliver energy to different regions of the tip.

16. The apparatus of claim 15, further comprising a shaft coupled to the tip, wherein the shaft comprises a first axis, wherein the spot coagulator tip is configured to pivot about the base relative to shaft, wherein the spot coagulator shaft comprises a second axis, and wherein the spot coagulator shaft is angled towards the first axis such that the second axis intersects the first axis.

17. The apparatus of claim 16, wherein the spot coagulator tip intersects the first axis.

18. The apparatus of claim 6, wherein the spot coagulator tip is configured to deliver energy at a position adjacent to a distal end of the tip.

19. The apparatus of claim 6, further comprising a first conductive element and a second conductive element, wherein the first conductive element is configured to electrically couple to the second conductive element in an operational configuration to allow the spot coagulator tip to deliver energy for coagulating a blood vessel, and wherein the first conductive element is electrically insulated from the second conductive element in a storage configuration to prevent the spot coagulator tip from delivering energy.

20. The apparatus of claim 19, wherein the spot coagulator is configured to provide for a plurality of operational positions such that at least one conductive element of the spot coagulator couples with at least one other conductive element such that the spot coagulator can deliver energy for coagulating a blood vessel at any of the plurality of operational positions.

21. The apparatus of claim 20, further comprising a plurality of conductive elements, wherein the second conductive element is one of the plurality of conductive elements, and wherein each of the conductive elements corresponds to one of the plurality of operational positions.

22. The apparatus of claim 20, wherein at least one of the first and second conductive elements comprises an insulator and a plurality of conducting sections, wherein each of the conducting sections comprises an opening in the insulator to allow the conducting sections to make contact with another conductive element, and wherein each of the conducting sections corresponds to one of the plurality of operational positions.

23. The apparatus of claim 19, wherein the spot coagulator tip is axially movable with respect to the shaft such that the spot coagulator can be advanced to the operational configuration and retracted to the storage configuration.

24. The apparatus of claim 19, wherein at least one of the first conductive element and the second conductive element is spring-biased to facilitate contact between the first conductive element and the second conductive element in the operational configuration.

25. The apparatus of claim 6, further comprising a shield configured to protect the spot coagulator tip.

26. The apparatus of claim 25, wherein the spot coagulator tip is configured to be positioned in a storage position and moved to an operational position, and wherein the shield is configured to protect the spot coagulator tip and prevent the spot coagulator tip from contacting tissue during an operation while the spot coagulator tip is in the storage position.

27. The apparatus of claim 6, further comprising a means for removably coupling the tip with a shaft of a surgical tool.

28. The apparatus of claim 27, wherein the means for removably coupling the tip with a shaft of a surgical tool comprises a plug configured to be received within a recess of the surgical tool.

29. The apparatus of claim 27, wherein the surgical tool comprises an endoscope.

30. The apparatus of claim 6, wherein the shaft comprises a conductive core.

31. The apparatus of claim 30, wherein the shaft is configured to be coupled with a surgical device.

32. The apparatus of claim 31, wherein the shaft comprises a protruding conductive element, and wherein the protruding conductive element is configured to contact the spot coagulator to receive electrical energy for delivering to the spot coagulator tip.

33. The apparatus of claim 32, wherein the protruding conductive element is spring-biased.

34. An apparatus for tissue separation and modification, comprising:
a tip comprising a plurality of protrusions;
at least one lysing element positioned between at least two adjacent protrusions in the plurality of protrusions;
a shaft coupled with the tip; and
a spot coagulator comprising:
   a spot coagulator shaft;
   a spot coagulator tip configured to deliver electrical energy for coagulating a blood vessel during a surgical procedure, wherein the spot coagulator tip is movable with respect to the plurality of protrusions; and
   a spot coagulator toggle coupled with the shaft and configured to move at least a portion of the spot coagulator with respect to the tip between an operational position and a storage position.

35. The apparatus of claim 34, wherein the spot coagulator is configured to electrically couple with a source of electrical energy in the operational position and to electrically decouple from the source of electrical energy in the storage position.

36. The apparatus of claim 34, wherein the spot coagulator toggle comprises a grip configured to facilitate movement between the operational position and the storage position.

37. The apparatus of claim 34, wherein the spot coagulator toggle is configured to move the at least a portion of the spot coagulator with respect to the tip between a plurality of operational positions and a storage position.

38. An apparatus for tissue separation and modification, comprising:
a tip comprising a plurality of protrusions;
at least one lysing element positioned between at least two adjacent protrusions in the plurality of protrusions;
a shaft coupled with the tip, wherein the shaft comprises a conductive core, wherein the shaft is configured to be coupled with a surgical device, and wherein the shaft comprises a protruding conductive element; and
a spot coagulator comprising:
   a spot coagulator shaft; and
   a spot coagulator tip configured to deliver electrical energy for coagulating a blood vessel during a surgical procedure, wherein the spot coagulator is configured to engage the protruding conductive element to receive energy from the surgical device to allow the spot coagulator tip to deliver electrical energy.

39. The apparatus of claim 38, wherein the protruding conductive element is at least one of spring-biased and spring loaded.

40. The apparatus of claim 38, wherein the protruding conductive element comprises a protruding fin.

41. The apparatus of claim 38, wherein the spot coagulator tip is movable with respect to the plurality of protrusions.

42. The apparatus of claim 38, further comprising a spot coagulator toggle coupled with the shaft and configured to move at least a portion of the spot coagulator with respect to the tip.

43. The apparatus of claim 38, wherein the tip comprises an internal tissue optical motion sensor comprising an electromagnetic emission source and a photodetector device, wherein the electromagnetic emission source is configured to deliver electromagnetic radiation, wherein the photodetector device is configured to receive reflected electromagnetic radiation from the electromagnetic emission source, and wherein the internal tissue optical motion sensor is configured to use the reflected electromagnetic radiation to detect movement of the tip relative to patient tissue during a surgical procedure.

44. A bipolar electrosurgical device for tissue separation and modification,
comprising:
a tip comprising a plurality of protrusions;
a plurality of lysing elements, wherein each of the lysing elements is positioned between two adjacent protrusions in the plurality of protrusions, and wherein each of the lysing elements is coupled to a lead configured to deliver electrical energy, and wherein each of the lysing elements is coupled to a lead having a voltage of a polarity opposite polarity relative to a lead coupled to an adjacent lysing element; and
a bipolar plug configured to be coupled with an electrosurgical generator, wherein the bipolar plug comprises a positive terminal and a negative terminal.

45. The bipolar electrosurgical device of claim 44, further comprising a bipolar circuit activating foot-pedal bypass configured to facilitate hand control of the bipolar electrosurgical device.

46. The bipolar electrosurgical device of claim 45, wherein the bipolar circuit activating foot-pedal bypass comprises a sticker configured to be adhered to the bipolar electrosurgical device.

47. The bipolar electrosurgical device of claim 45, wherein the bipolar circuit activating foot-pedal bypass comprises a sheath configured to be positioned over at least a portion of the bipolar electrosurgical device, wherein the sheath is configured to at least partially expose the tip after the sheath has been positioned over the at least a portion of the bipolar electrosurgical device.

48. The bipolar electrosurgical device of claim 45, wherein the bipolar circuit activating foot-pedal bypass comprises a first switch coupled to a first electrical lead and a second switch coupled to a second electrical lead, and wherein the first and second electrical leads are configured to be coupled to a foot pedal control to bypass the foot pedal control.

49. The bipolar electrosurgical device of claim 44, wherein the tip comprises a number of protrusions equal to the number of lysing elements.

50. The bipolar electrosurgical device of claim 44, further comprising a spot coagulator comprising:
a spot coagulator shaft; and
a spot coagulator tip configured to deliver electrical energy for coagulating a blood vessel during a surgical procedure.

51. The bipolar electrosurgical device of claim 44, further comprising a return electrode positioned on the tip.

52. The bipolar electrosurgical device of claim 51, wherein the return electrode comprises a band extending around a perimeter of the tip.

53. A surgical system, comprising:
a surgical device comprising:
   a tip comprising a plurality of protrusions;
   at least one lysing element positioned between at least two adjacent protrusions in the plurality of protrusions; and
   a shaft coupled with the tip, wherein the shaft is configured to be received in a shaft recess of an electrosurgical device;
a spot coagulator comprising:
   a spot coagulator shaft; and
   a spot coagulator tip configured to deliver electrical energy for coagulating a blood vessel during a surgical procedure; and
   means for selectively moving the spot coagulator relative to the tip.

54. The surgical system of claim 53, wherein the means for selectively moving the spot coagulator comprises a means for rigidly affixing the means for selectively moving the spot coagulator to the electrosurgical device.

55. The surgical system of claim 54, wherein the means for selectively moving the spot coagulator comprises a slidable toggle coupled with the spot coagulator shaft, wherein the slidable toggle is configured to axially advance the spot coagulator tip between a retracted position and at least one operational position.

56. The surgical system of claim 55, wherein the slidable toggle is configured to axially advance the spot coagulator tip between a retracted position and a plurality of discrete, operational positions.

57. The surgical system of claim 53, wherein the means for selectively moving the spot coagulator is configured to be selectively applied to the electrosurgical device.

58. The surgical system of claim 53, wherein the means for selectively moving the spot coagulator is coupled with the shaft of the surgical device.

59. The surgical system of claim 58, wherein the shaft of the surgical device comprises a conductive core, wherein the means for selectively moving the spot coagulator comprises a rod encasing a lead electrically coupled to the conductive core, and wherein the lead is electrically coupled with the spot coagulator tip.

60. The surgical system of claim 59, wherein the means for selectively moving the spot coagulator further comprises a first conductive element, wherein the spot coagulator shaft further comprises a second conductive element, wherein the first conductive element is configured to selectively electrically couple with the second conductive element in an operational position, and wherein the first conductive element is configured to selectively electrically decouple from the second conductive element in a storage position.

61. The surgical system of claim 53, wherein the tip comprises at least one tip passageway, and wherein the spot coagulator is configured to extend through the at least one tip passageway such that the spot coagulator tip can extend distally beyond the plurality of protrusions to deliver electrical energy for coagulating a blood vessel during a surgical procedu

62. The surgical system of claim 61, wherein the at least one tip passageway extends from the tip adjacent to at least one of the lysing elements.

63. The surgical system of claim 61, wherein the tip comprises a distal end surface and two opposing side surfaces, and wherein the at least one tip passageway extends from one of the side surfaces.

64. The surgical system of claim 53, wherein the surgical device comprises an internal tissue optical motion sensor configured to track movement of the surgical device relative to an internal tissue surface during a surgical procedure.

65. The surgical system of claim 64, wherein the internal tissue optical motion sensor comprises:
an electromagnetic emission source; and
a photodetector device, wherein the electromagnetic emission source is configured to deliver electromagnetic radiation, wherein the photodetector device is configured to receive reflected electromagnetic radiation from the electromagnetic emission source, and wherein the internal tissue optical motion sensor is configured to use the reflected electromagnetic radiation to detect movement of the tip relative to the internal tissue surface during the surgical procedure.

66. The surgical system of claim 64, wherein the internal tissue optical motion sensor comprises:
a non-coherent light source configured to illuminate the internal tissue surface;
a two-dimensional array of photodetectors configured to generate a photodetector signal in response to receiving light from the non-coherent light source; and
circuitry to receive the signal and track movement of the surgical device relative to the internal tissue surface by comparing a first photodetector signal at a first time with a second photodetector signal at a second time and comparing differences between the first photodetector signal and the second photodetector signal.

67. The surgical system of claim 53, further comprising a spot coagulator coupler configured to receive the spot coagulator therethrough.

68. The surgical system of claim 67, wherein the spot coagulator coupler is an integral part of the tip.

69. The surgical system of claim 53, further comprising:
a robotic arm coupled with the surgical device such that the tip is positioned at a distal end of the robotic arm.

70. The surgical system of claim 69, wherein the robotic arm comprises a plurality of arm segments with at least one joint positioned in between each two adjacent arm segments.

71. A surgical apparatus for tissue separation and modification, comprising:
a tip comprising a plurality of protrusions and a spot coagulator coupler comprising at least one passageway;
at least one lysing element positioned between at least two adjacent protrusions in the plurality of protrusions; and
a spot coagulator comprising:
   a spot coagulator shaft; and
   a spot coagulator tip configured to deliver electrical energy for coagulating a blood vessel during a surgical procedure, wherein the spot coagulator is configured to engage the protruding conductive element to receive energy from the surgical device to allow the spot coagulator tip to deliver electrical energy, and wherein the spot coagulator extends through the at least one passageway.

72. The surgical apparatus of claim 71, wherein the spot coagulator coupler is an integral part of the tip.

73. The surgical apparatus of claim 71, further comprising a shaft coupled with the tip.

74. The surgical apparatus of claim 73, wherein the shaft comprises a conductive core.

75. The surgical apparatus of claim 74, where the conductive core comprises a lysing element plate, and wherein the at least one lysing element is part of the lysing element plate.

76. The surgical apparatus of claim 71, further comprising a toggle coupled with the spot coagulator, wherein the toggle is configured to move at least a portion of the spot coagulator with respect to the tip between at least one operational position and at least one storage position.

77. The surgical apparatus of claim 76, wherein the spot coagulator is configured to move the at least a portion of the spot coagulator axially with respect to an axis of the surgical apparatus between the at least one operational position and the at least one storage position.

78. The surgical apparatus of claim 71, wherein the spot coagulator coupler comprises a sheath, wherein the spot coagulator tip is configured to be received in the sheath in a storage position, and wherein the spot coagulator tip is configured to extend out of the sheath in an operational position.

79. The surgical apparatus of claim 78, wherein the spot coagulator is configured such that the spot coagulator tip can receive electrical energy in the operational position and such that the spot coagulator tip is prevented from receiving electrical energy in the storage position.

It will be apparent to those having skill in the art that changes may be made to the details of the above-described embodiments without departing from the underlying principles set forth herein. The scope of the present invention should, therefore, be determined only by the following claims.

1. A surgical system, comprising:
a surgical device comprising:
a tip comprising a plurality of protrusions;
at least one lysing element positioned between at least two adjacent protrusions in the plurality of protrusions; and
a shaft coupled with the tip, wherein the shaft is configured to be received in a shaft recess of an electrosurgical device;
a spot coagulator comprising:
a spot coagulator shaft; and
a spot coagulator tip configured to deliver electrical energy for coagulating a blood vessel during a surgical procedure; and
means for selectively moving the spot coagulator relative to the tip.

2. The surgical system of claim 1, wherein the means for selectively moving the spot coagulator comprises a slidable toggle coupled with the spot coagulator shaft, wherein the slidable toggle is configured to axially advance the spot coagulator tip between a retracted position and at least one operational position.

3. The surgical system of claim 2, wherein the slidable toggle is configured to axially advance the spot coagulator tip between a retracted position and a plurality of discrete, operational positions.

4. The surgical system of claim 1, wherein the means for selectively moving the spot coagulator further comprises a first conductive element, wherein the spot coagulator shaft further comprises a second conductive element, wherein the first conductive element is configured to selectively electrically couple with the second conductive element in an operational position, and wherein the first conductive element is configured to selectively electrically decouple from the second conductive element in a storage position.

5. The surgical system of claim 1, further comprising a spot coagulator coupler configured to receive the spot coagulator therethrough.

6. The surgical system of claim 5, wherein the spot coagulator coupler is an integral part of the tip.

7. A surgical apparatus for tissue separation and modification, comprising:
a tip comprising a plurality of protrusions and a spot coagulator coupler comprising at least one passageway;
at least one lysing element positioned between at least two adjacent protrusions in the plurality of protrusions; and
a spot coagulator comprising:
a spot coagulator shaft; and
a spot coagulator tip configured to deliver electrical energy for coagulating a blood vessel during a surgical procedure, wherein the spot coagulator is configured to engage the protruding conductive element to receive energy from the surgical device to allow the spot coagulator tip to deliver electrical energy, and wherein the spot coagulator extends through the at least one passageway.

8. The surgical apparatus of claim 7, further comprising a shaft coupled with the tip, wherein the shaft comprises a conductive core.

9. The surgical apparatus of claim 8, where the conductive core comprises a lysing element plate, and wherein the at least one lysing element is part of the lysing element plate.

10. The surgical apparatus of claim 7, further comprising a toggle coupled with the spot coagulator, wherein the toggle is configured to move at least a portion of the spot coagulator with respect to the tip between at least one operational position and at least one storage position.

11. The surgical apparatus of claim 10, wherein the spot coagulator is configured to move the at least a portion of the spot coagulator axially with respect to an axis of the surgical apparatus between the at least one operational position and the at least one storage position.

12. The surgical apparatus of claim 7, wherein the spot coagulator coupler comprises a sheath, wherein the spot coagulator tip is configured to be received in the sheath in a storage position, and wherein the spot coagulator tip is configured to extend out of the sheath in an operational position.

13. The surgical apparatus of claim 7, wherein the spot coagulator is configured such that the spot coagulator tip can receive electrical energy in the operational position and such that the spot coagulator tip is prevented from receiving electrical energy in the storage position.

14. An apparatus for tissue separation and modification, comprising:
a tip comprising a plurality of protrusions;
at least one lysing element positioned between at least two adjacent protrusions in the plurality of protrusions; and
a spot coagulator comprising:
a spot coagulator shaft; and
a spot coagulator tip configured to deliver energy for coagulating a blood vessel during a surgical procedure with the apparatus for tissue separation and modification, wherein the spot coagulator tip is movable with respect to the plurality of protrusions.

15. The apparatus of claim 14, wherein the spot coagulator tip is configured to pivot about a base to selectively deliver energy to different regions of the tip.

16. The apparatus of claim 14, further comprising a first conductive element and a second conductive element, wherein the first conductive element is configured to electrically couple to the second conductive element in an operational configuration to allow the spot coagulator tip to deliver energy for coagulating a blood vessel, and wherein the first conductive element is electrically insulated from the second conductive element in a storage configuration to prevent the spot coagulator tip from delivering energy.

17. The apparatus of claim 16, wherein the spot coagulator is configured to provide for a plurality of operational positions such that at least one conductive element of the spot coagulator couples with at least one other conductive element such that the spot coagulator can deliver energy for coagulating a blood vessel at any of the plurality of operational positions.

18. The apparatus of claim 17, further comprising a plurality of conductive elements, wherein the second conductive element is one of the plurality of conductive elements, and wherein each of the conductive elements corresponds to one of the plurality of operational positions.

19. The apparatus of claim 17, wherein at least one of the first and second conductive elements comprises an insulator and a plurality of conducting sections, wherein each of the conducting sections comprises an opening in the insulator to allow the conducting sections to make contact with another conductive element, and wherein each of the conducting sections corresponds to one of the plurality of operational positions.

20. The apparatus of claim 16, wherein the spot coagulator tip is axially movable with respect to the shaft such that the spot coagulator can be advanced to the operational configuration and retracted to the storage configuration.
